SEQUENCE LISTING (11 sequences)

SEQ ID NO: 1 - protein, 278 aa, Homo sapiens
GTSMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQE

SEQ ID NO: 2 - protein, 278 aa, Homo sapiens
GTSMDPSSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQE

SEQ ID NO: 3 - protein, 2031 aa, Homo sapiens [sequence omitted]

SEQ ID NO: 4 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 5 - protein, 1530 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDAEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 6 - protein, 2031 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSKALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 7 - protein, 2006 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDADGSFGTPPGYGCAADRAEEQRRHQDGLPYIDDSPSSSPHLSSKGRGSRDALVSGALKSTKASELDLEKGLEMRKWVLSGILASEETYLSHLEALLLPMKPLKAAATTSQPVLTSQQIETIFFKVPELYEIHKESYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTDLLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 8 - protein, 1530 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGIMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDDEGAFHGDAEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 9 - protein, 676 aa, Homo sapiens
LGYWKIKGLVQPTRLLLEYLEEKYEEHLYERDEGDKWRNKKFELGLEFPNLPYYIDGDVKLTQSMAIIRYIADKHNMLGGCPKERAEISMLEGAVDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIWPLQGWQATFGGGDHPPKSDLVPRHNQTSLYKKAGSAAAVLEENLYFQGTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 10 - protein, 474 aa, Homo sapiens
MSYYHHHHHHDYDIPTTENLYFQGAMLVPRGSPWIPFTMKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 11 - protein, 12 aa, Unknown
  Abl kinase peptide substrate
EAIYAAPFAKKK

(19) United States
(12) Patent Application Publication
(10) Pub. No.: US 2011/0189167 A1
(43) Pub. Date: Aug. 4, 2011

(54) Methods and Compositions for the Treatment of Myeloproliferative Diseases and other Proliferative Diseases

(76) Inventors: Daniel L. FLYNN, Lawrence, KS (US); Peter A. PETILLO, Lawrence, KS (US); Michael D. KAUFMAN, Lawrence, KS (US)

(21) Appl. No.: 12/850,256

(22) Filed: Aug. 4, 2010

Related U.S. Application Data
(63) Continuation-in-part of application No. 12/105,408, filed on Apr. 18, 2008.
(60) Provisional application No. 60/913,216, filed on Apr. 20, 2007.

(51) Int. Cl. A61K 39/395; A61K 31/454; A61K 31/506; A61K 31/519; A61P 35/00; A61P 19/00; A61P 11/00

(52) U.S. Cl. 424/133.1; 514/341; 514/252.18; 514/275; 514/252.19; 514/253.06; 514/249; 424/142.1

(57) ABSTRACT

Methods of modulating a kinase activity of a wild-type kinase species, oncogenic forms thereof, aberrant fusion proteins thereof and polymorphs of any of the foregoing, are provided which employ compounds of the formula Ia:

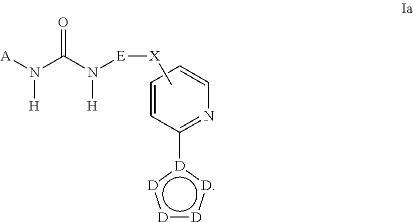

### CROSS-REFERENCE TO RELATED APPLICATIONS

This application claims the benefit of application Ser. No. 12/105,408 filed Apr. 18, 2008, which claims the benefit of Provisional Application 60/913,216 filed Apr. 20, 2007, the contents of both of which are incorporated by reference herein in their entirety.

### FIELD OF THE INVENTION

The present invention relates to novel kinase inhibitors and modulator compounds useful for the treatment of various diseases. More particularly, the invention is concerned with such compounds, methods of treating diseases, and methods of synthesis of the compounds. Preferably, the compounds are useful for the modulation of kinase activity of c-ABL, c-KIT, VEGFR, PDGFR, FLT-3, c-MET, FGFR, the HER family, cFMS, RET, oncogenic forms thereof, disease causing polymorphs thereof, and aberrant fusion proteins thereof.

### BACKGROUND OF THE INVENTION

Several members of the protein kinase family have been clearly implicated in the pathogenesis of various proliferative and myeloproliferative diseases and thus represent important targets for treatment of these diseases. Some of the proliferative diseases relevant to this invention include cancer, rheumatoid arthritis, atherosclerosis, and retinopathies. Important examples of kinases which have been shown to cause or contribute to the pathogenesis of these diseases include c-ABL kinase and the oncogenic fusion protein BCR-ABL kinase; c-KIT kinase, c-MET, the HER family of kinases, PDGF receptor kinases; VEGF receptor kinases; FLT-3 kinase, RET kinase, and c-FMS kinase.

c-ABL kinase is an important non-receptor tyrosine kinase involved in cell signal transduction. This ubiquitously expressed kinase—upon activation by upstream signaling factors including growth factors, oxidative stress, integrin stimulation, and ionizing radiation—localizes to the cell plasma membrane, the cell nucleus, and other cellular compartments including the actin cytoskeleton (Van Etten, Trends Cell Biol. (1999) 9: 179). There are two normal isoforms of ABL kinase: ABL-1A and ABL-1B. The N-terminal half of c-ABL kinase is important for autoinhibition of the kinase domain catalytic activity (Pluk et al, Cell (2002) 108: 247). Details of the mechanistic aspects of this autoinhibition have recently been disclosed (Nagar et al, Cell (2003) 112: 859). The N-terminal myristolyl amino acid residue of ABL-1B has been shown to intramolecularly occupy a hydrophobic pocket formed from alpha-helices in the C-lobe of the kinase domain. Such intramolecular binding induces a novel binding area for intramolecular docking of the SH2 domain and the SH3 domain onto the kinase domain, thereby distorting and inhibiting the catalytic activity of the kinase. Thus, an intricate intramolecular negative regulation of the kinase activity is brought about by these N-terminal regions of c-ABL kinase. An aberrant dysregulated form of c-ABL is formed from a chromosomal translocation event, referred to as the Philadelphia chromosome (P. C. Nowell et al, Science (1960) 132: 1497; J. D. Rowley, Nature (1973) 243: 290). This abnormal chromosomal translocation leads aberrant gene fusion between the ABL kinase gene and the breakpoint cluster region (BCR) gene, thus encoding an aberrant protein called BCR-ABL (G. Q. Daley et al, Science (1990) 247: 824; M. L. Gishizky et al, Proc. Natl. Acad. Sci. USA (1993) 90: 3755; S. Li et al, J. Exp. Med. (1999) 189: 1399). The BCR-ABL fusion protein does not include the regulatory myristolylation site (B. Nagar et al, Cell (2003) 112: 859) and as a result functions as an oncoprotein which causes chronic myeloid leukemia (CML). CML is a malignancy of pluripotent hematopoietic stem cells. The p210 form of BCR-ABL is seen in 95% of patients with CML, and in 20% of patients with acute lymphocytic leukemia and is exemplified by sequences such as e14a2 and e13a2. The corresponding p190 form, exemplified by the sequence el a2 has also been identified. A p185 form has also been disclosed and has been linked to being causative of up to 10% of patients with acute lymphocytic leukemia. It will be appreciated by one skilled in the art that “p210 form”, “p190 form” and “p185 form” each describe a closely related group of fusion proteins, and that Sequence ID's used herein are merely representative of each form and are not meant to restrict the scope solely to those sequences.

c-KIT (KIT, CD117, stem cell factor receptor) is a 145 kDa transmembrane tyrosine kinase protein that acts as a type-III receptor (Pereira et al. J Carcin. (2005), 4: 19). The c-KIT proto-oncogene, located on chromosome 4q11-21, encodes the c-KIT receptor, whose ligand is the stem cell factor (SCF, steel factor, c-KIT ligand, mast cell growth factor, Morstyn G, et al. Oncology (1994) 51(2):205. Yarden Y, et al. Embo J (1987) 6(11):3341). The receptor has tyrosine-protein kinase activity and binding of the ligands leads to the autophosphorylation of c-KIT and its association with substrates such as phosphatidylinositol 3-kinase (Pi3K). Tyrosine phosphorylation by protein tyrosine kinases is of particular importance in cellular signaling and can mediate signals for major cellular processes, such as proliferation, differentiation, apoptosis, attachment, and migration. Defects in c-KIT are a cause of piebaldism, an autosomal dominant genetic developmental abnormality of pigmentation characterized by congenital patches of white skin and hair that lack melanocytes. Gain-of-function mutations of the c-KIT gene and the expression of phosphorylated c-KIT are found in most gastrointestinal stromal tumors and mastocytosis. Further, almost all gonadal seminomas/dysgerminomas exhibit c-KIT membranous staining, and several reports have clarified that some (10-25%) have a c-KIT gene mutation (Sakuma, Y. et al. Cancer Sci (2004) 95:9, 716). C-KIT defects have also been associated with testicular tumors including germ cell tumors (GCT) and testicular germ cell tumors (TGCT).

The role of c-KIT expression has been studied in hematologic and solid tumors, such as acute leukemias (Cortes J. et al. Cancer (2003) 97(11):2760) and gastrointestinal stromal tumors (GIST, Fletcher C. D. et al. Hum Pathol (2002) 33(5):459). The clinical importance of c-KIT expression in malignant tumors relies on studies with Gleevec® (imatinib mesylate, STI571, Novartis Pharma AG Basel, Switzerland) that specifically inhibits tyrosine kinase receptors (Lefevre G. et al. J Biol Chem (2004) 279(30):31769). Moreover, a clinically relevant breakthrough has been the finding of anti-tumor effects of this compound in GIST, a group of tumors regarded as being generally resistant to conventional chemotherapy (de Silva C M, Reid R: Pathol Oncol Res (2003) 9(1):13-19). GIST most often become Gleevec resistant and molecularly targeted small therapies that target c-KIT mutations remain elusive.

c-MET is a unique receptor tyrosine kinase (RTK) located on chromosome 7p and activated via its natural ligand hepatocyte growth factor. c-MET is found mutated in a variety of solid tumors (Ma P. C. et al. Cancer Metastasis (2003) 22:309). Mutations in the tyrosine kinase domain are associated with hereditary papillary renal cell carcinomas (Schmidt L et al. Nat. Genet. (1997)16:68; Schmidt L, et al. Oncogene (1999) 18:2343), whereas mutations in the sema and juxtamembrane domains are often found in small cell lung cancers (SCLC; Ma P. C. et al. Cancer Res (2003) 63:6272). Many activating mutations are also found in breast cancers (Nakopoulou et al. Histopath (2000) 36(4): 313). The panoply of tumor types for which c-MET mediated growth has been implicated suggests this is a target ideally suited for modulation by specific c-MET small molecule inhibitors.

The TPR-MET oncogene is a transforming variant of the c-MET RTK and was initially identified after treatment of a human osteogenic sarcoma cell line transformed by the chemical carcinogen N-methyl-N-nitro-N-nitrosoguanidine (Park M. et al. Cell (1986) 45:895). The TPR-MET fusion oncoprotein is the result of a chromosomal translocation, placing the TPR3 locus on chromosome 1 upstream of a portion of the c-MET gene on chromosome 7 encoding only for the cytoplasmic region. Studies suggest that TPR-MET is detectable in experimental cancers (e.g. Yu J. et al. Cancer (2000) 88:1801). Dimerization of the Mᵣ 65,000 TPR-MET oncoprotein through a leucine zipper motif encoded by TPR leads to constitutive activation of the c-MET kinase (Zhen Z. et al. Oncogene (1994) 9:1691). TPR-MET activates wild-type c-MET RTK and can activate crucial cellular growth pathways, including the Ras pathway (Aklilu F. et al. Am J Physiol (1996) 271:E277) and the phosphatidylinositol 3-kinase (PI3K)/AKT pathway (Ponzetto C. et al. Mol Cell Biol (1993) 13:4600). Conversely, in contrast to c-MET RTK, TPR-MET is ligand independent, lacks the CBL binding site in the juxtamembrane region in c-MET, and is mainly cytoplasmic. c-MET immunohistochemical expression seems to be associated with abnormal β-catenin expression, and provides good prognostic and predictive factors in breast cancer patients.

The majority of small molecule kinase inhibitors that have been reported have been shown to bind in one of three ways. Most of the reported inhibitors interact with the ATP binding domain of the active site and exert their effects by competing with ATP for occupancy. Such inhibitors are referred to as Type 1 kinase inhibitors. Other inhibitors have been shown to bind to a separate hydrophobic region of the protein known as the “DFG-in-conformation” pocket, and still others have been shown to bind to both the ATP domain and the “DFG-in-conformation” pocket. The latter two types of kinase inhibitors are referred to as Type II kinase inhibitors. Some of the kinase inhibitors of the present invention are Type II inhibitors. Examples specific to inhibitors of Raf kinases can be found in Lowinger et al, Current Pharmaceutical Design (2002) 8: 2269-2278; Dumas, J. et al., Current Opinion in Drug Discovery & Development (2004) 7: 600-616; Dumas, J. et al, WO 2003068223 A1 (2003); Dumas, J., et al, WO 9932455 A1 (1999), and Wan, P. T. C., et al, Cell (2004) 116: 855-867.

Physiologically, kinases are regulated by a common activation/deactivation mechanism wherein a specific activation loop sequence of the kinase protein binds into a specific pocket on the same protein which is referred to as the switch control pocket (see WO 2004061084 and WO 2007008917 for further details). Such binding occurs when specific amino acid residues of the activation loop are modified for example by phosphorylation, oxidation, or nitrosylation. The binding of the activation loop into the switch pocket results in a conformational change of the protein into its active form (Huse, M. and Kuriyan, J. Cell (109) 275-282). Some of the inhibitors of the present invention induce kinases to adopt inactive conformations through inhibitor binding at least in part into the switch control pocket.

### BRIEF SUMMARY OF THE INVENTION

Compounds of the present invention find utility in the treatment of hyperproliferative diseases, including autoimmune diseases and other diseases characterized by hypervascularization or proliferation of myeloid, mast cells, fibroblasts, synoviocytes, or monocytes; mammalian cancers and especially human cancers including but not limited to melanomas; a disease caused by c-ABL kinase, oncogenic forms thereof, aberrant fusion proteins thereof including BCR-ABL kinase and polymorphs thereof a disease caused by FLT-3 kinase, oncogenic forms thereof, aberrant fusion proteins thereof and polymorphs thereof a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof a disease caused by RET kinase, oncogenic forms thereof, aberrant fusion proteins thereof a disease caused by c-FMS kinase, oncogenic forms thereof and polymorphs thereof a disease caused by a c-KIT kinase, oncogenic forms thereof, aberrant fusion proteins thereof and polymorphs thereof and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, papillary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonary disease, bone resorptive diseases, bone cancer, graft-versus-host reaction, Chron's disease, ulcerative colitis, inflammatory bowel disease, pyresis, gastrointestinal stromal tumors, mastocytosis, mast cell leukemia, and combinations thereof.

### DETAILED DESCRIPTION OF THE INVENTION

The following descriptions refer to various compounds, stereo-, regioisomers and tautomers of such compounds and individual moieties of the compounds thereof.

Cycloalkyl refers to monocyclic saturated carbon rings taken from cyclopropyl, cyclobutyl, cyclopentyl, cyclohexyl, cycloheptanyl, and cyclooctanyl;

Aryl refers to monocyclic or fused bicyclic ring systems characterized by delocalized it electrons (aromaticity) shared among the ring carbon atoms of at least one carbocyclic ring; preferred aryl rings are taken from phenyl, naphthyl, tetrahydronaphthyl, indenyl, and indanyl;
Heteroaryl refers to monocyclic or fused bicyclic ring systems characterized by delocalized π electrons (aromaticity) shared among the ring carbon or heteroatoms including nitrogen, oxygen, or sulfur of at least one carbocyclic or heterocyclic ring; heteroaryl rings are taken from, but not limited to, pyrrolyl, furyl, thienyl, oxazolyl, thiazolyl, isoxazolyl, isothiazolyl, imidazolyl, pyrazolyl, oxadiazolyl, thiadiazolyl, triazolyl, tetrazolyl, pyridinyl, pyrimidinyl, pyrazinyl, pyridazinyl, triazinyl, indolyl, indolinyl, isoindolyl, isoindolinyl, indazolyl, benzofuranyl, benzothienyl, benzothiazolyl, benzothiazolonyl, benzoxazolyl, benzoxazolonyl, benzisoxazolyl, benzisothiazolyl, benzimidazolyl, benzimidazolonyl, benztriazolyl, imidazopyridinyl, pyrazolopyridinyl, imidazolonopyridinyl, thiazolopyridinyl, thiazolonopyridinyl, oxazolopyridinyl, oxazolonopyridinyl, isoxazolopyridinyl, isothiazolopyridinyl, triazolopyridinyl, imidazopyrimidinyl, pyrazolopyrimidinyl, imidazolonopyrimidinyl, thiazolopyridiminyl, thiazolonopyrimidinyl, oxazolopyridiminyl, oxazolonopyrimidinyl, isoxazolopyrimidinyl, isothiazolopyrimidinyl, triazolopyrimidinyl, dihydropurinonyl, pyrrolopyrimidinyl, purinyl, pyrazolopyrimidinyl, phthalimidyl, phthalimidinyl, pyrazinylpyridinyl, pyridinopyrimidinyl, pyrimidinopyrimidinyl, cinnolinyl, quinoxalinyl, quinazolinyl, quinolinyl, isoquinolinyl, phthalazinyl, benzodioxyl, benzisothiazo line-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl;
Heterocyclyl refers to monocyclic rings containing carbon and heteroatoms taken from oxygen, nitrogen, or sulfur and wherein there is not delocalized π electrons (aromaticity) shared among the ring carbon or heteroatoms; heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
Poly-aryl refers to two or more monocyclic or fused aryl bicyclic ring systems characterized by delocalized π electrons (aromaticity) shared among the ring carbon atoms of at least one carbocyclic ring wherein the rings contained therein are optionally linked together;
Poly-heteroaryl refers to two or more monocyclic or fused bicyclic systems characterized by delocalized π electrons (aromaticity) shared among the ring carbon or heteroatoms including nitrogen, oxygen, or sulfur of at least one carbocyclic or heterocyclic ring wherein the rings contained therein are optionally linked together, wherein at least one of the monocyclic or fused bicyclic rings of the poly-heteroaryl system is taken from heteroaryl as defined broadly above and the other rings are taken from either aryl, heteroaryl, or heterocyclyl as defined broadly above;
Poly-heterocyclyl refers to two or more monocyclic or fused bicyclic ring systems containing carbon and heteroatoms taken from oxygen, nitrogen, or sulfur and wherein there is not delocalized π electrons (aromaticity) shared among the ring carbon or heteroatoms wherein the rings contained therein are optionally linked, wherein at least one of the monocyclic or fused bicyclic rings of the poly-heteroaryl system is taken from heterocyclyl as defined broadly above and the other rings are taken from either aryl, heteroaryl, or heterocyclyl as defined broadly above;
Alkyl refers to straight or branched chain C1-C6alkyls;
Halogen refers to fluorine, chlorine, bromine, and iodine;
Alkoxy refers to —O-(alkyl) wherein alkyl is defined as above;
Alkoxylalkyl refers to -(alkyl)-O-(alkyl) wherein alkyl is defined as above;
Alkoxylcarbonyl refers to —C(O)O-(alkyl) wherein alkyl is defined as above;
Carboxyl C1-C6alkyl refers to —(C1-C6)alkyl wherein alkyl is defined as above;
Substituted in connection with a moiety refers to the fact that a further substituent may be attached to the moiety to any acceptable location on the moiety.

The term salts embraces pharmaceutically acceptable salts commonly used to form alkali metal salts of free acids and to form addition salts of free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, and heterocyclyl containing carboxylic acids and sulfonic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, 2-hydroxyethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid. Suitable pharmaceutically-acceptable salts of free acid-containing compounds of the invention include metallic salts and organic salts. More preferred metallic salts include, but are not limited to appropriate alkali metal (group Ia) salts, alkaline earth metal (group IIa) salts and other physiological acceptable metals. Such salts can be made from aluminum, calcium, lithium, magnesium, potassium, sodium and zinc. Preferred organic salts can be made from primary amines, secondary amines, tertiary amines and quaternary ammonium salts, including in part, tromethamine, diethylamine, tetra-N-methylammonium, N,N′-dibenzylethylenediamine, chloroprocaine, choline, diethanolamine, ethylenediamine, meglumine (N-methylglucamine) and procaine.

The term prodrug refers to derivatives of active compounds which revert in vivo into the active form. For example, a carboxylic acid form of an active drug may be esterified to create a prodrug, and the ester is subsequently converted in vivo to revert to the carboxylic acid form. See Ettmayer et. al, J. Med. Chem., 2004, 47 (10): 2393-2404 and Lorenzi et. al, J. Pharm. Exp. Therapeutics, 2005, 883-900 for reviews.

Structural, chemical and stereochemical definitions are broadly taken from IUPAC recommendations, and more specifically from Glossary of Terms used in Physical Organic Chemistry (IUPAC Recommendations 1994) as summarized by P. Müller, Pure Appl. Chem., 66, 1077-1184 (1994) and Basic Terminology of Stereochemistry (IUPAC Recommendations 1996) as summarized by G. P. Moss Pure and Applied Chemistry, 68, 2193-2222 (1996). Specific definitions are as follows: Atropisomers are defined as a subclass of conformers which can be isolated as separate chemical species and which arise from restricted rotation about a single bond.

Regioisomers or structural isomers are defined as isomers involving the same atoms in different arrangements.

Enantiomers are defined as one of a pair of molecular entities which are mirror images of each other and non-superimposable.

Diastereomers or diastereoisomers are defined as stereoisomers other than enantiomers. Diastereomers or diastereoisomers are stereoisomers not related as mirror images. Diastereoisomers are characterized by differences in physical properties, and by some differences in chemical behavior towards achiral as well as chiral reagents.

Tautomerism is defined as isomerism of the general form

G-X—Y═Z [symbol] X═Y—Z-G

where the isomers (called tautomers) are readily interconvertible; the atoms connecting the groups X, Y, Z are typically any of C, H, O, or S, and G is a group which becomes an electrofuge or nucleofuge during isomerization. The commonest case, when the electrofuge is H⁺, is also known as “prototropy”.

Tautomers are defined as isomers that arise from tautomerism, independent of whether the isomers are isolable.

### First Aspect of the Invention—Compounds, Methods, Preparations and Adducts

The invention includes compounds of the formula Ia:

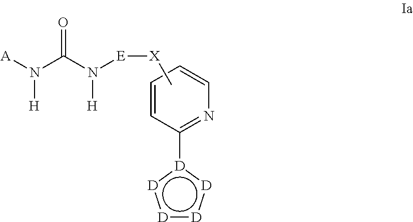

and wherein the pyridine ring may be optionally substituted with one or more R20 moieties;
each D is individually taken from the group consisting of C, CH, C—R20, N—Z3, and N, such that the resultant ring is a pyrazole;
wherein E is selected from the group consisting of phenyl, pyridyl, and pyrimidinyl;
E may be optionally substituted with one or two R16 moieties;
wherein A is a ring system selected from the group consisting of phenyl, naphthyl, cyclopentyl, cyclohexyl, G1, G2, and G3;
G1 is a heteroaryl taken from the group consisting of pyrrolyl, furyl, thienyl, oxazolyl, thiazolyl, isoxazol-4-yl, isoxazol-5-yl, isothiazolyl, imidazolyl, pyrazolyl, oxadiazolyl, thiadiazolyl, triazolyl, tetrazolyl, pyrazinyl, pyridazinyl, triazinyl, pyridinyl, and pyrimidinyl;
G2 is a fused bicyclic heteroaryl taken from the group consisting of indolyl, indolinyl, isoindolyl, isoindolinyl, indazolyl, benzofuranyl, benzothienyl, benzothiazolyl, benzothiazolonyl, benzoxazolyl, benzoxazolonyl, benzisoxazolyl, benzisothiazolyl, benzimidazolyl, benzimidazolonyl, benztriazolyl, imidazopyridinyl, pyrazolopyridinyl, imidazolonopyridinyl, thiazolopyridinyl, thiazolonopyridinyl, oxazolopyridinyl, oxazolonopyridinyl, isoxazolopyridinyl, isothiazolopyridinyl, triazolopyridinyl, imidazopyrimidinyl, pyrazolopyrimidinyl, imidazolonopyrimidinyl, thiazolopyridiminyl, thiazolonopyrimidinyl, oxazolopyridiminyl, oxazolonopyrimidinyl, isoxazolopyrimidinyl, isothiazolopyrimidinyl, triazolopyrimidinyl, dihydropurinonyl, pyrrolopyrimidinyl, purinyl, pyrazolopyrimidinyl, phthalimidyl, phthalimidinyl, pyrazinylpyridinyl, pyridinopyrimidinyl, pyrimidinopyrimidinyl, cinnolinyl, quinoxalinyl, quinazolinyl, quinolinyl, isoquinolinyl, phthalazinyl, benzodioxyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl;
G3 is a heterocyclyl taken from the group consisting of oxetanyl, azetadinyl, tetrahydrofuranyl, pyrrolidinyl, oxazolinyl, oxazolidinyl, imidazolonyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
the A ring may be optionally substituted with one or two R2 moieties;
X is selected from the group consisting of —O—, —S(CH₂)ₙ—, —N(R3)(CH₂)ₙ—, —(CH₂)ₚ—, and wherein the carbon atoms of —(CH₂)ₙ—, —(CH₂)ₚ—, of X may be further substituted by oxo or one or more C1-C6alkyl moieties;
when A, G1, G2 or G3 has one or more substitutable sp2-hybridized carbon atoms, each respective sp2 hybridized carbon atom may be optionally substituted with a Z1 substituent;
when A, G1, G2 or G3 has one or more substitutable sp3-hybridized carbon atoms, each respective sp3 hybridized carbon atom may be optionally substituted with a Z2 substituent;
when A, G1, G2 or G3 has one or more substitutable nitrogen atoms, each respective nitrogen atom may be optionally substituted with a Z4 substituent;
each Z1 is independently and individually selected from the group consisting of C1-6alkyl, branched C3-C7alkyl, C3-C8cycloalkyl, halogen, fluoroC1-C6alkyl wherein the alkyl moiety can be partially or fully fluorinated, cyano, C1-C6alkoxy, fluoroC1-C6alkoxy wherein the alkyl moiety can be partially or fully fluorinated, —(CH₂)ₙOH, oxo, C1-C6alkoxyC1-C6alkyl, (R4)₂N(CH₂)ₙ—, (R3)₂N(CH₂)ₙ—, (R4)₂N(CH₂)_{q}N(R4)(CH₂)ₙ—, (R4)₂N(CH₂)_{q}O(CH₂)ₙ—, (R3)₂NC(O)—, (R4)₂NC(O)—, (R4)₂NC(O)C1-C6alkyl-, —(R4)NC(O)R8, C1-C6alkoxycarbonyl-, -carboxyC1-C6alkyl, C1-C6alkoxycarbonylC1-C6alkyl-, (R3)₂NSO₂—, —SOR3, (R4)₂NSO₂—, —N(R4)SO₂R8, —O(CH₂)_{q}OC1-C6alkyl, —SO₂R3, —SOR4, —C(O)R8, —C(O)R6, —C(═NOH)R6, —C(═NOR3)R6, —(CH₂)ₙN(R4)C(O)R8, —N(R3)(CH₂)_{q}O-alkyl, —N(R3)(CH₂)_{q}N(R4)₂, nitro, —CH(OH)CH(OH)R4, —C(═NH)N(R4)₂, —C(═NOR3)N(R4)₂, and —NHC(═NH)R8, R17 substituted G3, R17 substituted pyrazolyl and R17 substituted imidazolyl;
in the event that Z1 contains an alkyl or alkylene moiety, such moieties may be further substituted with one or more C1-C6alkyls;

each Z2 is independently and individually selected from the group consisting of aryl, C1-C6alkyl, C3-C8cycloalkyl, branched C3-C7alkyl, hydroxyl, hydroxyC1-C6alkyl-, cyano, (R3)₂N—, (R4)₂N—, (R4)₂NC1-C6alkyl-, (R4)₂NC2-C₆alkylN(R4)(CH₂)ₙ—, (R4)₂NC2-C6alkylO(CH₂)ₙ—, (R3)₂NC(O)—, (R4)₂NC(O)—, (R4)₂NC(O)—C1-C6alkyl-, carboxyl, -carboxyC1-C6alkyl, C1-C6alkoxycarbonyl-, C1-C6alkoxycarbonylC1-C6alkyl-, (R3)₂NSO₂—, (R4)₂NSO₂—, —SO₂R8, —(CH₂)ₙN(R4)C(O)R8, —C(O)R8, ═O, ═NOH, and ═N(OR6);

in the event that Z2 contains an alkyl or alkylene moiety, such moieties may be further substituted with one or more C1-C6alkyls;
each Z3 is independently and individually selected from the group consisting of H, C1-C6alkyl, branched C3-C7alkyl, C3-C8cycloalkyl, fluoroC1-C6alkyl wherein the alkyl moiety can be partially or fully fluorinated, hydroxyC2-C6alkyl-, C1-C6alkoxycarbonyl-, —C(O)R8, R5C(O)(CH₂)ₙ—, (R4)₂NC(O)—, (R4)₂NC(O)C1-C6alkyl-, R8C(O)N(R4)(CH₂)_{q}—, (R3)₂NSO₂—, (R4)₂NSO₂—, —(CH₂)_{q}N(R3)₂, and —(CH₂)_{q}N(R4)₂;
each Z4 is independently and individually selected from the group consisting of C1-C6alkyl, branched C₃₋₇alkyl, hydroxyC2-C6alkyl-, C1-C6alkoxyC2-C6alkyl-, (R4)₂N—C2-C6alkyl-, (R4)₂N—C2-C6alkylN(R4)-C2-C6alkyl-, (R4)₂N—C2-C6alkyl-O—C2-C6alkyl-(R4)₂NC(O)C1-C6alkyl-, carboxyC1-C6alkyl, C1-C6alkoxycarbonylC1-C6alkyl-, —C2-C6alkylN(R4)C(O)R8, R8-C(═NR3)-, —SO₂R8, and —COR8;
in the event that Z4 contains an alkyl or alkylene moiety, such moieties may be further substituted with one or more C1-C6alkyls;
each R2 is selected from the group consisting of H, C1-C6alkyl, branched C3-C8alkyl, R19 substituted C3-C8cycloalkyl-, fluoroC1-C6alkyl- wherein the alkyl is fully or partially fluorinated, halogen, cyano, C1-C6alkoxy-, and fluoroC1-C6alkoxy- wherein the alkyl group is fully or partially fluorinated, hydroxyl substituted C1-C6alkyl-, hydroxyl substituted branched C3-C8alkyl-, cyano substituted C1-C6alkyl-, cyano substituted branched C3-C8 alkyl-, (R3)₂NC(O)C1-C6 alkyl-, (R3)₂NC(O)C3-C8 branched alkyl-;
wherein each R3 is independently and individually selected from the group consisting of H, C1-C6alkyl, branched C3-C7alkyl, and C3-C8cycloalkyl;
each R4 is independently and individually selected from the group consisting of H, C1-C6 alkyl, hydroxyC1-C6alkyl-, dihydroxyC1-C6alkyl-, C1-C6 alkoxyC1-C6 alkyl-, branched C3-C7 alkyl, branched hydroxyC1-C6 alkyl-, branched C1-C6 alkoxyC1-C6alkyl-, branched dihydroxyC1-C6alkyl-, —(CH₂)ₚN(R7)₂, —(CH₂)ₚC(O)N(R7)₂, —(CH₂)ₙC(O)OR3, R19 substituted C3-C8 cyclo alkyl-;
each R5 is independently and individually selected from the group consisting of

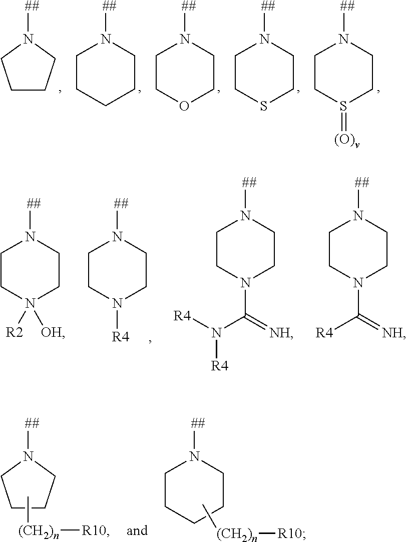

and wherein the symbol (##) is the point of attachment to Z3;
each R6 is independently and individually selected from the group consisting of C1-C6alkyl, branched C3-C7alkyl, and R19 substituted C3-C8cycloalkyl-;
each R7 is independently and individually selected from the group consisting of H, C1-C6alkyl, hydroxyC2-C6alkyl-, dihydroxyC2-C6alkyl-, C1-C6alkoxyC2-C6alkyl-, branched C3-C7alkyl, branched hydroxyC2-C6alkyl-, branched C1-C6alkoxyC2-C6alkyl-, branched dihydroxyC2-C6alkyl-, —(CH₂)ₙC(O)OR3, R19 substituted C3-C8 cyclo alkyl- and —(CH₂)ₙR17;
each R8 is independently and individually selected from the group consisting of C1-C6alkyl, branched C3-C7alkyl, fluoroC1-C6alkyl- wherein the alkyl moiety is partially or fully fluorinated, R19 substituted C3-C8cycloalkyl-, —OH, C1-C6alkoxy, —N(R3)₂, and —N(R4)₂;
each R10 is independently and individually selected from the group consisting of —CO₂H, —CO₂C1-C6alkyl, —C(O)N(R4)₂, OH, C1-C6alkoxy, and —N(R4)₂;
each R16 is independently and individually selected from the group consisting of H, C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, R3 substituted C2-C3alkynyl- and nitro;
each R17 is independently and individually selected from the group consisting of H, C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, and nitro;
each R19 is independently and individually selected from the group consisting of H, OH and C1-C6alkyl;
each R20 is independently and individually selected from the group consisting of C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, —N(R3)C(O)R3, —C(O)N(R3)₂ and nitro and wherein two R4 moieties independently and individually taken from the group consisting of C1-C6alkyl, branched C3-C6alkyl, hydroxyalkyl-, and alkoxyalkyl and attached to the same nitrogen heteroatom may cyclize to form a C3-C7 heterocyclyl ring;
and k is 0 or 1; n is 0-6; p is 1-4; q is 2-6; r is 0 or 1; t is 1-3; v is 1 or 2; m is 0-2; and stereo-, regioisomers and tautomers of such compounds.
1.1 Compounds of Formula Ia which Exemplify Preferred D Moieties

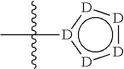

In a preferred embodiment of compounds of formula Ia, said compounds have preferred

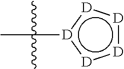

moieties of the formula:

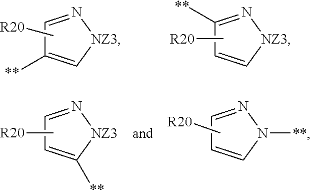

wherein the symbol (**) indicates the point of attachment to the pyridine ring.
1.1.1 Compounds of Formula Ia which Exemplify Preferred A Moieties

In a preferred embodiment of compounds of formula Ia, said compounds have structures of formula Ib

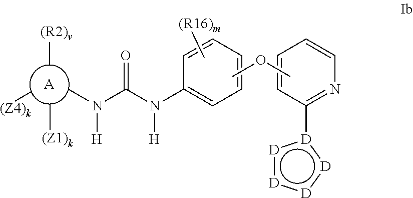

wherein A is any possible isomer of pyrazole.
1.1.2 Compounds of Formula Ia which Exemplify Preferred A and R16 Moieties

In a more preferred embodiment of compounds of formula Ib, said compounds have structures of formula Ic

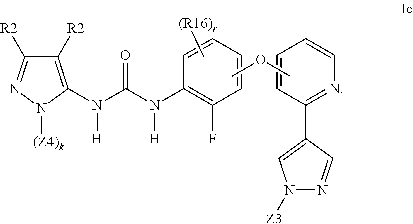

1.1.3 Compounds of Formula Ia which Exemplify Preferred A and R16 Moieties

In a more preferred embodiment of compounds of formula Ib, said compounds have structures of formula Id

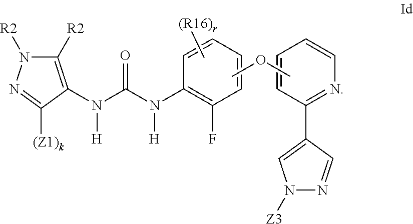

1.1.4 Compounds of Formula Ia which Exemplify Preferred A and R16 Moieties

In a more preferred embodiment of compounds of formula Ib, said compounds have structures of formula Ie

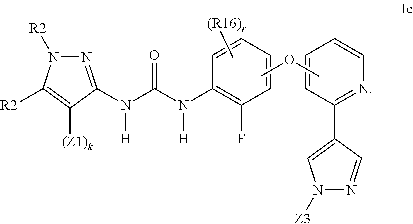

1.1.5 Compounds of Formula Ia which Exemplify Preferred A and R16 Moieties

In a more preferred embodiment of compounds of formula Ia, said compounds have structures of formula If

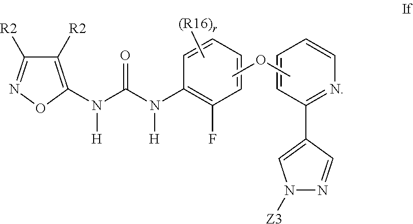

1.1.6 Compounds of Formula Ia which Exemplify Preferred A Moieties

In a preferred embodiment of compounds of formula Ia, said compounds have structures of formula Ig

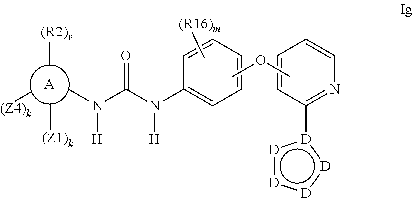

wherein A is selected from the group consisting of any isomer of phenyl and pyridine.
1.1.7 Compounds of Formula Ia which Exemplify Preferred A and R16 Moieties

In a more preferred embodiment of compounds of formula Ig, said compounds have structures of formula Ih

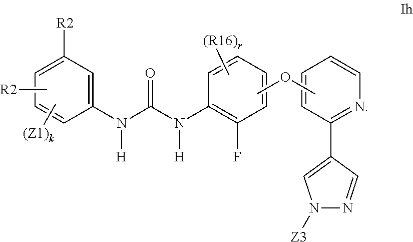

1.1.8 Compounds of Formula Ia which Exemplify Preferred A and R16 Moieties

In a more preferred embodiment of compounds of formula Ig, said compounds have structures of formula Ii

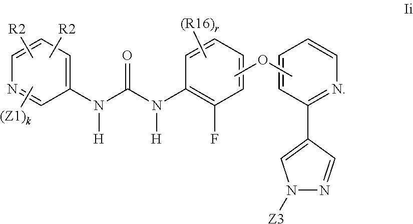

1.1.9 Compounds of Formula Ia which Exemplify Preferred A Moieties

In a preferred embodiment of compounds of formula Ia, said compounds have structures of formula Ij

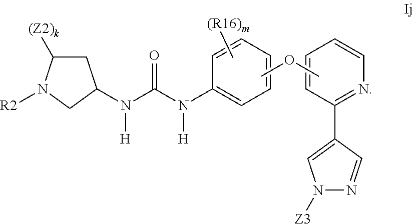

1.1.10 Compounds of Formula Ia which Exemplify Preferred A and R16 Moieties

In a more preferred embodiment of compounds of formula Ia, said compounds have structures of formula Ik

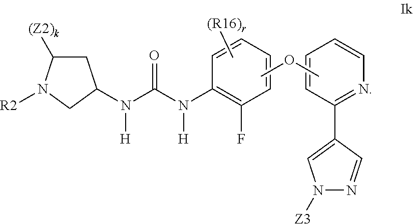

### 1.1.11 Most Preferred Compounds of Formula Ia

1-(3-tert-butylisoxazol-5-yl)-3-(2-fluoro-4-(2-(1-methyl-1H-pyrazol-4-yl)pyridin-4-yloxy)phenyl)urea, 1-(3-tert-butyl-1-methyl-1H-pyrazol-5-yl)-3-(2-fluoro-4-(2-(1-methyl-1H-pyrazol-4-yl)pyridin-4-yloxy)phenyl)urea, 1-(3-tert-butylisoxazol-5-yl)-3-(3-methyl-4-(2-(1-methyl-1H-pyrazol-4-yl)pyridin-4-yloxy)phenyl)urea, 1-(3-tert-butyl-1-methyl-1H-pyrazol-5-yl)-3-(3-methyl-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-(trifluoromethyl)phenyl)urea, 1-(4-(2-(1-methyl-1H-pyrazol-4-yl)pyridin-4-yloxy)phenyl)-3-(3-(trifluoromethyl)phenyl)urea, 1-(5-tert-butylisoxazol-3-yl)-3-(2-fluoro-4-(2-(1-methyl-1H-pyrazol-4-yl)pyridin-4-yloxy)phenyl)urea, 1-(5-tert-butylisoxazol-3-yl)-3-(4-(2-(1-methyl-1H-pyrazol-4-yl)pyridin-4-yloxy)phenyl)urea, 1-(1-tert-butyl-1H-pyrazol-4-yl)-3-(2-fluoro-4-(2-(1-methyl-1H-pyrazol-4-yl)pyridin-4-yloxy)phenyl)urea, 1-(4-(2-(1-methyl-1H-pyrazol-4-yl)pyridin-4-yloxy)phenyl)-3-(5-(trifluoromethyl)pyridin-3-yl)urea, 1-(4-chloro-3-(trifluoromethyl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(5-isopropylisoxazol-3-yl)urea, 1-(2,3-difluorophenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-(trifluoromethyl)isoxazol-5-yl)urea, 1-(1-tert-butyl-1H-pyrazol-4-yl)-3-(2,3-difluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-isopropylisoxazol-5-yl)urea, 1-(1-tert-butyl-5-(trifluoromethyl)-1H-pyrazol-4-yl)-3-(2-fluoro-4-(2-(1-methyl-1H-pyrazol-4-yl)pyridin-4-yloxy)phenyl)urea, 1-(1-tert-butyl-5-methyl-1H-pyrazol-4-yl)-3-(2-fluoro-4-(2-(1-methyl-1H-pyrazol-4-yl)pyridin-4-yloxy)phenyl)urea, 1-(5-tert-butyl-1,3,4-thiadiazol-2-yl)-3-(2-fluoro-4-(2-(1-methyl-1H-pyrazol-4-yl)pyridin-4-yloxy)phenyl)urea, 1-(3,5-dichlorophenyl)-3-(2-fluoro-4-(2-(1-methyl-1H-pyrazol-4-yl)pyridin-4-yloxy)phenyl)urea, 1-cyclohexyl-3-(2-fluoro-4-(2-(1-methyl-1H-pyrazol-4-yl)pyridin-4-yloxy)phenyl)urea, 1-(3-tert-butyl-1-(2-(dimethylamino)ethyl)-1H-pyrazol-5-yl)-3-(2-fluoro-4-(2-(1-methyl-1H-pyrazol-4-yl)pyridin-4-yloxy)phenyl)urea, 1-cyclopentyl-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(1-isopropyl-1H-pyrazol-4-yl)urea, 1-(2-fluoro-4-(2-(1-methyl-1H-pyrazol-4-yl)pyridin-4-yloxy)phenyl)-3-(3-(1-methylcyclopentyl)isoxazol-5-yl)urea, 1-(4-chlorophenyl)-3-(2-fluoro-4-(2-(1-methyl-1H-pyrazol-4-yl)pyridin-4-yloxy)phenyl)urea, 1-(3-cyclopentylisoxazol-5-yl)-3-(2-fluoro-4-(2-(1-methyl-1H-pyrazol-4-yl)pyridin-4-yloxy)phenyl)urea, 1-(1-cyclopentyl-1H-pyrazol-4-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(1-methyl-3-(1-methylcyclopentyl)-1H-pyrazol-5-yl)urea, 1-(2-fluoro-4-(2-(1-methyl-1H-pyrazol-4-yl)pyridin-4-yloxy)phenyl)-3-(1-methyl-3-(trifluoromethyl)-1H-pyrazol-5-yl)urea, 1-(2-fluoro-4-(2-(1-methyl-1H-pyrazol-4-yl)pyridin-4-yloxy)phenyl)-3-(2-fluoro-5-(trifluoromethyl)phenyl)urea, 1-(3-tert-butylphenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(2-fluoro-5-methylphenyl)urea, 1-(1-tert-butyl-1H-pyrazol-4-yl)-3-(2-fluoro-3-methyl-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-isopropylphenyl)urea, 1-(1-tert-butyl-1H-pyrazol-4-yl)-3-(3-fluoro-4-(2-(1-methyl-1H-pyrazol-4-yl)pyridin-4-yloxy)phenyl)urea, 1-(5-fluoro-2-methylphenyl)-3-(2-fluoro-4-(2-(1-methyl-1H-pyrazol-4-yl)pyridin-4-yloxy)phenyl)urea, 1-(3-cyclopentyl-1-methyl-1H-pyrazol-5-yl)-3-(2-fluoro-4-(2-(1-methyl-1H-pyrazol-4-yl)pyridin-4-yloxy)phenyl)urea, 1-(1-tert-butyl-1H-pyrazol-4-yl)-3-(2-fluoro-4-(2-(1-propyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-fluorophenyl)urea, 1-(2-fluoro-4-(2-(1-methyl-1H-pyrazol-4-yl)pyridin-4-yloxy)phenyl)-3-(1-isopropyl-3-(trifluoromethyl)-1H-pyrazol-4-yl)urea, 1-(2-fluoro-3-methyl-4-(2-(1-methyl-1H-pyrazol-4-yl)pyridin-4-yloxy)phenyl)-3-(1-isopropyl-1H-pyrazol-4-yl)urea, 1-(2-fluoro-4-(2-(1-methyl-1H-pyrazol-4-yl)pyridin-4-yloxy)phenyl)-3-(1-isopropyl-5-methyl-1H-pyrazol-4-yl)urea, 1-(2-fluoro-4-(2-(1-methyl-1H-pyrazol-4-yl)pyridin-4-yloxy)phenyl)-3-(1-isopropyl-3-methyl-1H-pyrazol-4-yl)urea, 1-(2-fluoro-4-(2-(1-methyl-1H-pyrazol-4-yl)pyridin-4-yloxy)phenyl)-3-(5-(trifluoromethyl)pyridin-3-yl)urea, 1-cyclohexyl-3-(2,3-difluoro-4-(2-(1-methyl-1H-pyrazol-4-yl)pyridin-4-yloxy)phenyl)urea, 1-cyclohexyl-3-(2-fluoro-3-methyl-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(5-isopropylpyridin-3-yl)urea, 1-(1-cyclopentyl-5-methyl-1H-pyrazol-4-yl)-3-(2-fluoro-4-(2-(1-methyl-1H-pyrazol-4-yl)pyridin-4-yloxy)phenyl)urea, 1-(1-cyclopentyl-5-methyl-1H-pyrazol-4-yl)-3-(2,3-difluoro-4-(2-(1-methyl-1H-pyrazol-4-yl)pyridin-4-yloxy)phenyl)urea, 1-(benzo[d]isoxazol-3-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(5-fluoropyridin-3-yl)urea, 1-(3-cyanophenyl)-3-(2-fluoro-4-(2-(1-methyl-1H-pyrazol-4-yl)pyridin-4-yloxy)phenyl)urea, 1-(3-tert-butylisoxazol-5-yl)-3-(2,3-difluoro-4-(2-(1-methyl-1H-pyrazol-4-yl)pyridin-4-yloxy)phenyl)urea, 1-(3-tert-butylisoxazol-5-yl)-3-(2-fluoro-3-methyl-4-(2-(1-methyl-1H-pyrazol-4-yl)pyridin-4-yloxy)phenyl)urea, 1-(3-tert-butylisoxazol-5-yl)-3-(3-fluoro-4-(2-(1-methyl-1H-pyrazol-4-yl)pyridin-4-yloxy)phenyl)urea, 1-(2-tert-butyloxazol-5-yl)-3-(2-fluoro-4-(2-(1-methyl-1H-pyrazol-4-yl)pyridin-4-yloxy)phenyl)urea, 1-(3-tert-butyl-1-methyl-1H-pyrazol-5-yl)-3-(2,3-difluoro-4-(2-(1-methyl-1H-pyrazol-4-yl)pyridin-4-yloxy)phenyl)urea, 1-(3-tert-butyl-1-methyl-1H-pyrazol-5-yl)-3-(2-fluoro-3-methyl-4-(2-(1-methyl-1H-pyrazol-4-yl)pyridin-4-yloxy)phenyl)urea, 1-(1-cyclopentyl-1H-pyrazol-4-yl)-3-(2,3-difluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(2-oxo-5-(trifluoromethyl)-1,2-dihydropyridin-3-yl)urea, 1-(5-tert-butyl-2-methylfuran-3-yl)-3-(2-fluoro-4-(2-(1-methyl-1H-pyrazol-4-yl)pyridin-4-yloxy)phenyl)urea, 1-(2,3-difluoro-4-(2-(1-methyl-1H-pyrazol-4-yl)pyridin-4-yloxy)phenyl)-3-(3-isopropylisoxazol-5-yl)urea, 1-(2-fluoro-3-methyl-4-(2-(1-methyl-1H-pyrazol-4-yl)pyridin-4-yloxy)phenyl)-3-(3-isopropylisoxazol-5-yl)urea, 1-(2-fluoro-4-(2-(1-methyl-1H-pyrazol-4-yl)pyridin-4-yloxy)phenyl)-3-(6-fluorobenzo[d]thiazol-2-yl)urea, 1-(2-fluoro-3-methyl-4-(2-(1-methyl-1H-pyrazol-4-yl)pyridin-4-yloxy)phenyl)-3-(6-fluorobenzo[d]thiazol-2-yl)urea, 1-(1-tert-butyl-1H-pyrrol-3-yl)-3-(2-fluoro-4-(2-(1-methyl-1H-pyrazol-4-yl)pyridin-4-yloxy)phenyl)urea, 1-(3-tert-butyl-4-methylisoxazol-5-yl)-3-(2-fluoro-4-(2-(1-methyl-1H-pyrazol-4-yl)pyridin-4-yloxy)phenyl)urea, 1-(2-fluoro-3-methyl-4-(2-(1-methyl-1H-pyrazol-4-yl)pyridin-4-yloxy)phenyl)-3-(5-isopropylpyridin-3-yl)urea, 1-(2,3-difluoro-4-(2-(1-methyl-1H-pyrazol-4-yl)pyridin-4-yloxy)phenyl)-3-(5-isopropylpyridin-3-yl)urea, 1-(2-fluoro-4-(2-(1-methyl-1H-pyrazol-4-yl)pyridin-4-yloxy)phenyl)-3-(5-methylpyridin-3-yl)urea, 1-(2-fluoro-3-methyl-4-(2-(1-methyl-1H-pyrazol-4-yl)pyridin-4-yloxy)phenyl)-3-(5-(trifluoromethyl)pyridin-3-yl)urea, 1-(2,3-difluoro-4-(2-(1-methyl-1H-pyrazol-4-yl)pyridin-4-yloxy)phenyl)-3-(5-(trifluoromethyl)pyridin-3-yl)urea, 1-(5-ethylpyridin-3-yl)-3-(2-fluoro-4-(2-(1-methyl-1H-pyrazol-4-yl)pyridin-4-yloxy)phenyl)urea, 1-(5-chloropyridin-3-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-isopropyl-1-methyl-1H-pyrazol-5-yl)urea, 1-(3-cyclopropyl-1-methyl-1H-pyrazol-5-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(1-isopropyl-1H-imidazol-4-yl)urea, 1-(1-tert-butyl-5-oxopyrrolidin-3-yl)-3-(2-fluoro-4-(2-(1-methyl-1H-pyrazol-4-yl)pyridin-4-yloxy)phenyl)urea, 1-(1-tert-butylpyrrolidin-3-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(2-methyl-5-(trifluoromethyl)pyridin-3-yl)urea, 1-(2-tert-butyl-4-(piperazin-1-yl)pyrimidin-5-yl)-3-(2-fluoro-4-(2-(1-methyl-1H-pyrazol-4-yl)pyridin-4-yloxy)phenyl)urea, 1-(2-tert-butyl-4-morpholinopyrimidin-5-yl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(2-(1-methyl-1H-pyrazol-4-yl)-5-(trifluoromethyl)pyridin-3-yl)urea, and 1-(1-tert-butyl-5-methyl-1H-pyrazol-3-yl)-3-(2-fluoro-4-(2-(1-methyl-1H-pyrazol-4-yl)pyridin-4-yloxy)phenyl)urea.

### 1.2 Methods

### 1.2a Methods of Protein Modulation

The invention includes methods of modulating kinase activity of a variety of kinases, e.g. c-ABL kinase, BCR-ABL kinase, FLT-3, VEGFR-2 kinase mutants, c-MET, c-KIT, PDGFR kinases, the HER family of kinases, RET kinase, and c-FMS kinase. The kinases may be wildtype kinases, oncogenic forms thereof, aberrant fusion proteins thereof or polymorphs of any of the foregoing. The method comprises the step of contacting the kinase species with compounds of the invention and especially those set forth in sections section 1. The kinase species may be activated or unactivated, and the species may be modulated by phosphorylations, sulfation, fatty acid acylations glycosylations, nitrosylation, cystinylation (i.e. proximal cysteine residues in the kinase react with each other to form a disulfide bond) or oxidation. The kinase activity may be selected from the group consisting of catalysis of phospho transfer reactions, inhibition of phosphorylation, oxidation or nitrosylation of said kinase by another enzyme, enhancement of dephosphorylation, reduction or denitrosylation of said kinase by another enzyme, kinase cellular localization, and recruitment of other proteins into signaling complexes through modulation of kinase conformation.

### 1.2b Treatment Methods

The methods of the invention also include treating individuals suffering from a condition selected from the group consisting of cancer and hyperproliferative diseases. These methods comprise administering to such individuals compounds of the invention, and especially those of section 1, said diseases including, but not limited to, a disease caused by c-ABL kinase, oncogenic forms thereof, aberrant fusion proteins thereof including BCR-ABL kinase and polymorphs thereof; a disease caused by FLT-3 kinase, oncogenic forms thereof, aberrant fusion proteins thereof and polymorphs thereof; a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof; a disease caused by RET kinase, oncogenic forms thereof, aberrant fusion proteins thereof; a disease caused by c-FMS kinase, oncogenic forms thereof and polymorphs thereof; a disease caused by a c-KIT kinase, oncogenic forms thereof, aberrant fusion proteins thereof and polymorphs thereof; and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, papillary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonary disease, bone resorptive diseases, bone cancer, graft-versus-host reaction, Chron's disease, ulcerative colitis, inflammatory bowel disease, pyresis, gastrointestinal stromal tumors, mastocytosis, mast cell leukemia, and combinations thereof. The administration method is not critical, and may be from the group consisting of oral, parenteral, inhalation, and subcutaneous.

Dosage

The methods of the present invention may be used to prevent, treat, or reduce the severity of cancer or hyperproliferative diseases. The exact amount required will vary from subject to subject, depending on the species, age, and general condition of the subject, the severity of the disease, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, body surface area, general health, sex, ethnicity and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Administration of a compound of the invention or pharmaceutiacally active agent described herein can be accomplished via any mode of administration for therapeutic agents. These modes include systemic or local administration such as oral, nasal, parenteral, transdermal, subcutaneous, vaginal, buccal, rectal or topical administration modes. In some instances, administration will result in the release of the inhibitor or pharmaceutiacally active agent described herein into the bloodstream.

In one embodiment, the inhibitor or pharmaceutiacally active agent described herein is administered orally.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, preferably in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using dissolution or suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, aqueous dextrose, glycerol, ethanol, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous injection or intramuscular injection, or to slow the rate of systemic absorption upon oral administration. This may be accomplished by the use of a liquid suspension of crystalline or amorphous material with poor water solubility. The rate of absorption of the compound then depends upon its rate of dissolution that, in turn, may depend upon crystal size and crystalline form. Modified or sustained release formulations, well known in the art, may also be utilized in formulations to control the rate of absorption of an orally administered compound. Alternatively, modified or sustained absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders or diluents such as starches, lactose, sucrose, glucose, mannitol, cellulose, saccharin, glycine, and silicic acid, b) binders such as, for example, magnesium aluminum silicate, starch paste, tragacanth, carboxymethylcellulose, methyl cellulose, alginates, gelatin, polyvinylpyrrolidinone, magnesium carbonate, natural sugars, corn sweeteners, sucrose, waxes and natural or synthetic gums such as acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators or disintegrants such as quaternary ammonium compounds, starches, agar, methyl cellulose, bentonite, xanthangum, algiic acid, and effervescent mixtures, g) wetting agents such as, for example, cetyl alcohol and glycerol monostearate, h) absorbents such as kaolin and bentonite clay, and i) lubricants such as talc, silica, stearic acid, calcium stearate, magnesium stearate, sodium oleate, sodium acetate, sodium chloride, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a modified or sustained manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a modified or sustained manner. Examples of embedding compositions that can be used include polymeric substances and waxes.

The compound of the invention or pharmaceutically active agent described herein can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

The compound of the invention or pharmaceutically active agent described herein can also be delivered by the use of monoclonal antibodies as individual carriers to which the compound or pharmaceutiacally active agent described herein are coupled or conjugated. The compound or pharmaceutically active agent described herein can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the compound or pharmaceutically active agent described herein can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Furthermore, a compound or pharmaceutically active agent described herein may be coupled, absorbed, adsorbed, or conjugated to a medical device including but not limited to stents.

Parenteral injectable administration can be used for subcutaneous, intramuscular, intra-articular, or intravenous injections and infusions. Injectables can be prepared in conventional forms, either as liquid solutions or suspensions or solid forms suitable for dissolving in liquid prior to injection.

One embodiment, for parenteral administration employs the implantation of a slow-release or sustained-released system, according to U.S. Pat. No. 3,710,795, incorporated herein by reference.

The compositions can be sterilized or contain non-toxic amounts of adjuvants, such as preserving, stabilizing, wetting or emulsifying agents, solution promoters, salts for regulating the osmotic pressure, pH buffering agents, and other substances, including, but not limited to, sodium acetate or triethanolamine oleate. In addition, they can also contain other therapeutically valuable substances.

Dosage forms for topical or transdermal administration of a compound of this invention include ointments, pastes, creams, lotions, gels, powders, solutions, sprays, inhalants or patches. The compound or pharmaceutically active agent described herein is admixed under sterile conditions with a pharmaceutically acceptable carrier and any needed preservatives or buffers as may be required. Ophthalmic formulation, ear drops, and eye drops are also contemplated as being within the scope of this invention. Furthermore, the compound or pharmaceutically active agent described herein can be administered in intranasal form via topical use of suitable intranasal vehicles. Additionally, the present invention contemplates the use of transdermal patches or via other transdermal routes, using those forms of transdermal skin patches and formulations well known to those of ordinary skill in that art. Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compositions can be prepared according to conventional mixing, granulating or coating methods, respectively, and the present pharmaceutical compositions can contain from about 0.1% to about 99%, preferably from about 1% to about 70% of the compound or pharmaceutically active agent described herein by weight or volume.

The dosage regimen utilizing the compound of the invention or pharmaceutically active agent described herein can be selected in accordance with a variety of factors including type, species, age, weight, body surface area, sex, ethnicity, and medical condition of the subject; the severity of the condition to be treated; the route of administration; the renal or hepatic function of the subject; and the particular compound or pharmaceutically active agent described herein employed. A person skilled in the art can readily determine and prescribe the effective amount of the drug useful for treating or preventing a proliferative disorder.

Effective dosage amounts of the compound of the invention or pharmaceutically active agent described herein, when administered to a subject, range from about 0.05 to about 3,500 mg of compound or pharmaceutically active agent described herein per day. Unit dosage compositions for in vivo or in vitro use can contain about 0.01, 0.5, 1.0, 2.5, 5.0, 10.0, 15.0, 25.0, 50.0, 100.0, 250.0, 500.0 or 1000.0 mg of the compound described herein. In one embodiment, the unit dosage compositions are in the form of a tablet that can be scored. Effective plasma levels of the compound or pharmaceutically active agent described herein can be achieved from dosages from about 0.002 mg to about 50 mg per kg of body weight per day. The amount of a compound of the invention or pharmaceutically active agent described herein that is effective in the treatment or prevention of cancer or hyperproliferative disease can be determined by clinical techniques that are known to those of skill in the art. In addition, in vitro and in vivo assays can optionally be employed to help identify optimal dosage ranges. The precise dose to be employed can also depend on the route of administration, and the seriousness of the proliferative disorder being treated and can be decided according to the judgment of the practitioner and each subject's circumstances in view of, e.g., published clinical studies. Suitable effective dosage amounts, however, can range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one compound of the invention or pharmaceutiacally active agent described herein is administered, the effective dosage amounts correspond to the total amount administered.

The dosage regimen utilizing the compound of the invention or pharmaceutically active agent described herein can be selected in accordance with a variety of factors including type, species, age, weight, body surface area, sex, ethnicity, and medical condition of the subject; the severity of the cancer or hyperproliferative disorder to be treated; the route of administration; the renal or hepatic function of the subject; and the particular inhibitor or pharmaceutically active agent described herein employed. A person skilled in the art can readily determine and prescribe the effective amount of the drug required to prevent, counter or arrest the progress of the proliferative disorder.

The compound of the invention or pharmaceutically active agent described herein can be administered in a single daily dose, or the total daily dosage can be administered in divided doses of two, three or four times daily. When administered in the form of a transdermal delivery system, the dosage administration can be continuous rather than intermittent throughout the dosage regimen. Dosage strengths of topical preparations including creams, ointments, lotions, aerosol sprays and gels, contain the compound or pharmaceutiacally active agent described herein ranging from about 0.1% to about 15%, w/w or w/v.

Combination

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be administered simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the invention, an additional therapeutic agent, and a pharmaceutically acceptable carrier, adjuvant, or vehicle.

In certain embodiments, a combination of one additional agent and a compound of the invention are described. In some embodiments, two or more additional agents may be administered with a compound of the invention. In other embodiments, a combination of three or more additional agents may be administered with a compound of the invention. In some embodiments, the additional agent is selected from taxanes such as taxol, taxotere or their analogues; alkylating agents such as cyclophosphamide, isosfamide, melphalan, hexamethylmelamine, thiotepa or dacarbazine; antimetabolites such as pyrimidine analogues, for instance 5-fluorouracil, cytarabine, capecitabine, azacitibine, and gemcitabine or its analogues such as 2-fluorodeoxycytidine; folic acid analogues such as methotrexate, idatrexate or trimetrexate; spindle poisons including vinca alkaloids such as vinblastine, vincristine, vinorelbine and vindesine, or their synthetic analogues such as navelbine, or estramustine and a taxoid; platinum compounds such as cisplatin; epipodophyllotoxins such as etoposide or teniposide; steroids such as prednisone; antibiotics such as daunorubicin, doxorubicin, bleomycin or mitomycin, enzymes such as L-asparaginase, topoisomerase inhibitors such as topotecan or pyridobenzoindole derivatives; and various agents such as procarbazine, mitoxantrone; biological response modifiers or growth factor inhibitors such as interferons or interleukins; inhibitors of growth factors, for example Bevacizumab and Ranibizumab; kinase inhibitors including Cetuximab, Imatinib, Trastuzumab, Gefitinib, Pegaptanib, Sorafenib, Dasatinib, Bosutinib, AP-24534 also defined as 3-(2-(imidazo[1,2-b]pyridazin-3-yl)ethynyl)-4-methyl-N-(4-((4-methylpiperazin-1-yl)methyl)-3-(trifluoromethyl)phenyl)benzamide, Sunitinib, Erlotinib, Nilotinib, Lapatinib, Panitumumab, Pazopanib, Crizotinib, the JAK inhibitor CP-690,550, and the SYK inhibitor Fostamatinib. In other embodiments, the other agent in addition to a compound of the invention is Imatinib.

Other examples of agents the compounds of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, methotrexate, azathioprine, cyclophosphamide, and sulfasalazine; TNF blockers including Humira®, Enbrel®, and Remicade®; IL-1 RA including Kineret® and Rilonacept; anti-CD20 agents including Rituxin®; immunomodulatory and immunosuppressive agents such as abatacept, cyclosporin, tacrolimus, rapamycin, mycophenolate mofetil, interferons, corticosteroids, cyclophophamide, azathioprine, and sulfasalazine; bone resorptive inhibitory agents including denosumab and bisphosphonates including zoledronic acid; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, compounds of the present invention, or a pharmaceutically acceptable composition thereof, are administered in combination with a monoclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the compositions comprise an amount of an anticancer inhibitor described herein, e.g., a kinase inhibitor, and another anticancer agent which together are effective to treat or prevent cancer. In another embodiment, the amount of the anticancer inhibitor described herein and another anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of the anticancer inhibitor described herein and another anticancer agent. Other compositions of the present invention are prepared so that a parenteral dosage unit contains from about 0.01% to about 2% by weight of the composition.

The present methods for treating or preventing cancer or a hyperproliferative disease in a subject in need thereof can further comprise administering another prophylactic or therapeutic agent to the subject being administered an anticancer inhibitor or an anti-proliferative inhibitor described herein. In one embodiment the other prophylactic or therapeutic agent is administered in an effective amount. The other prophylactic or therapeutic agent includes, but is not limited to, an anti-inflammatory agent, an anti-renal failure agent, an anti-diabetic agent, an anti-cardiovascular disease agent, an antiemetic agent, a hematopoietic colony stimulating factor, an anxiolytic agent, and an opioid or non-opioid analgesic agent.

In a further embodiment, the anticancer inhibitor described herein can be administered prior to, concurrently with, or after an antiemetic agent, or on the same day, or within 1 hour, 2 hours, 12 hours, 24 hours, 48 hours or 72 hours of each other.

In another embodiment, the anticancer inhibitor described herein can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still another embodiment, the anticancer inhibitor described herein can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the anticancer inhibitor described herein can be administered prior to, concurrently with, or after an anxiolytic agent, or on the same day, or within 1 hour, 2 hours, 12 hours, 24 hours, 48 hours or 72 hours of each other.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the anticancer compound or anti-proliferative compound described herein is less than its effective amount would be where the other therapeutic agent is not administered. In this case, without being bound by theory, it is believed that the anticancer compound or anti-proliferative compound described herein and the other therapeutic agent act synergistically to treat or prevent cancer or hyperproliferative disease.

Antiemetic agents useful in the methods of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, and tropisetron.

Hematopoietic colony stimulating factors useful in the methods of the present invention include, but are not limited to, filgrastim, sargramostim, molgramostim and epoietin alfa.

Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

Non-opioid analgesic agents useful in the methods of the present invention include, but are not limited to, acetaminophen, acetaminophen plus codeine, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

Anxiolytic agents useful in the methods of the present invention include, but are not limited to, buspirone, and benzodiazepines such as diazepam, lorazepam, oxazapam, chlorazepate, clonazepam, chlordiazepoxide and alprazolam.

### 1.3 Pharmaceutical Preparations

The compounds of the invention, especially those of section 1 may form a part of a pharmaceutical composition by combining one or more such compounds with a pharamaceutically acceptable carrier. Additionally, the compositions may include an additive selected from the group consisting of adjuvants, excipients, diluents, and stabilizers.

### Section 2. Synthesis of Compounds of the Present Invention

The compounds of the invention are available by the procedures and teachings of WO 2006/071940, incorporated by reference, and by the general synthetic methods illustrated in the Schemes below and the accompanying examples.

As indicated in Scheme 1, ureas of general formula 1 can be readily prepared by the union of amines of general formula 2 with isocyanates 3 or isocyanate surrogates, for example trichloroethyl carbamates (4) or isopropenyl carbamates (5). Preferred conditions for the preparation of compounds of general formula 1 involve heating a solution of 4 or 5 with 2 in the presence of a tertiary base such as diisopropylethylamine, triethylamine or N-methylpyrrolidine in a solvent such as dimethylformamide, dimethylsulfoxide, tetrahydrofuran or 1,4-dioxane at a temperature between 50 and 100° C. for a period of time ranging from 1 hour to 2 days.

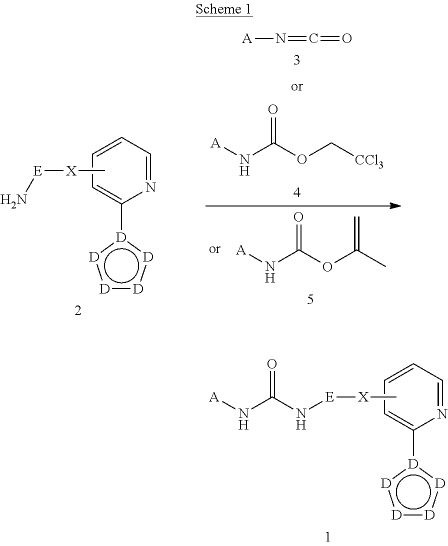

As shown in Scheme 2, isocyanates 3 can be prepared from amines A-NH₂ 6 with phosgene, or a phosgene equivalent such as diphosgene, triphosgene, or N,N-dicarbonylimidazole. Trichloroethyl carbamates 4 and isopropenyl carbamates 5 are readily prepared from amines A-NH₂ (6) by acylation with trichloroethyl chloroformate or isopropenyl chloroformate by standard conditions familiar to those skilled in the art. Preferred conditions for the preparation of 4 and 5 include include treatment of compound 6 with the appropriate chloroformate in the presence of pyridine in an aprotic solvent such as dichloromethane or in the presence of aqueous hydroxide or carbonate in a biphasic aqueous/ethyl acetate solvent system.

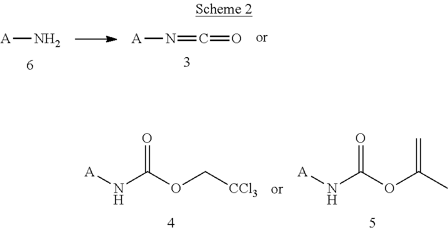

Additionally, compounds of formula 1 can also be prepared from carboxylic acids 7 by the intermediacy of in-situ generated acyl azides (Curtius rearrangement) as indicated in Scheme 3. Preferred conditions for Scheme 3 include the mixing of acid 7 with amine 2 and diphenylphosphoryl azide in a solvent such as 1,4-dioxane or dimethylformamide in the presence of base, such as triethylamine, and raising the temperature of the reaction to about 80-120° C. to affect the Curtius rearrangement.

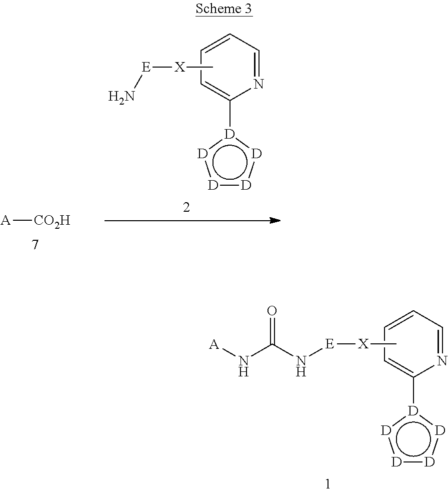

By analogy to Schemes 1 and 3 above, it will be recognized by those skilled in the art that the compounds of formula 1 can also be prepared by the union of amines A-NH₂ 6 with isocyanates 8 (Scheme 4). Isocyanates 8 can be prepared from general amines 2 by standard synthetic methods. Suitable methods for example, include reaction of 2 with phosgene, or a phosgene equivalent such as diphosgene, triphosgene, or N,N-dicarbonylimidazole. In addition to the methods above for converting amines 2 into isocynates 8, the isocyanates 8 can also be prepared in situ by the Curtius rearrangement and variants thereof. Those skilled in the art will further recognize that isocycanates 8 need not be isolated, but may be simply generated in situ. Accordingly, acid 9 can be converted to compounds of formula 1 either with or without isolation of 8. Preferred conditions for the direct conversion of acid 9 to compounds of formula 1 involve the mixing of acid 9, amine A-NH₂ 6, diphenylphosphoryl azide and a suitable base, for example triethylamine, in an aprotic solvent, for example dioxane. Heating said mixture to a temperature of between 80 and 120° C. provides the compounds of formula 1.

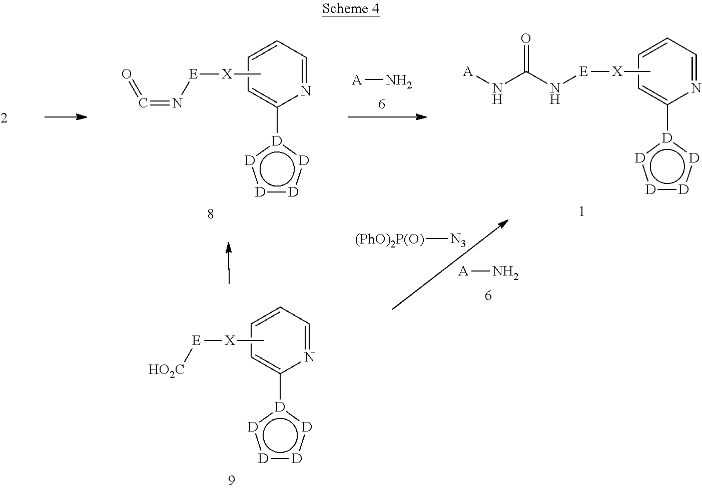

Additionally, compounds of formula 1 can also be prepared from amines 2 by first preparing stable isocyanate equivalents, such as carbamates (Scheme 5). Especially preferred carbamates include trichloroethyl carbamates (10) and isopropenyl carbamates (11) which are readily prepared from amine 2 by reaction with trichloroethyl chloroformate or isopropenyl chloroformate respectively using standard conditions familiar to those skilled in the art. Further reaction of carbamates 10 or 11 with amine A-NH₂ 6 provides compounds of formula 1. Those skilled in the art will further recognize that certain carbamates can also be prepared from acid 9 by Curtius rearrangement and trapping with an alcoholic co-solvent. For example, treatment of acid 9 (Scheme 5) with diphenylphosphoryl azide and trichloroethanol at elevated temperature provides trichloroethyl carbamate 10.

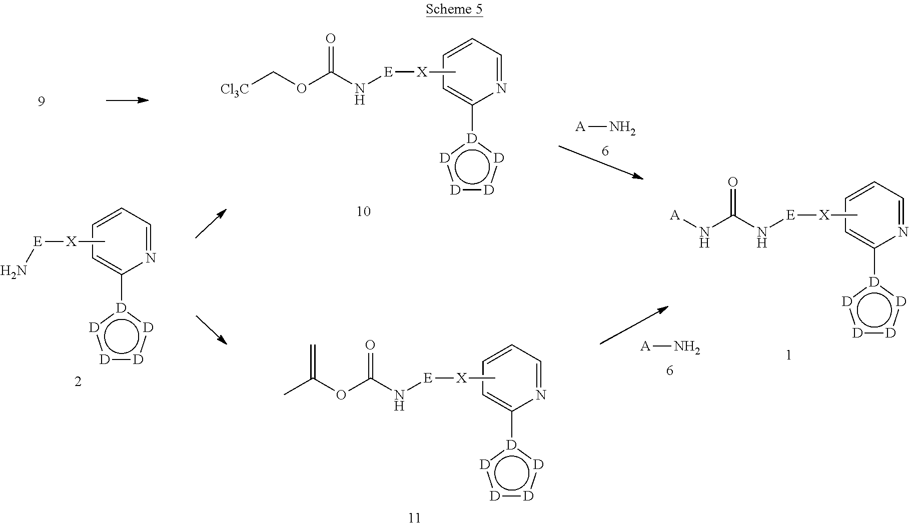

Many methods exist for the preparation of amines A-NH₂ 6 and acids A-CO₂H 7, depending on the nature of the A-moiety. Indeed, many such amines (6) and acids (7) useful for the preparation of compounds of formula 1 are available from commercial vendors. Some non-limiting preferred synthetic methods for the preparation of amines 6 and acids 7 are outlined in the following schemes and accompanying examples.

As illustrated in Scheme 6, Z4-substituted pyrazol-5-yl amines 14 (a preferred aspect of A-NH₂ 6, Scheme 2) are available by the condensation of hydrazines 12 and beta-keto nitriles 13 in the presence of a strong acid. Preferred conditions for this transformation are by heating in ethanolic HCl. Many such hydrazines 12 are commercially available. Others can be prepared by conditions familiar to those skilled in the art, for example by the diazotization of amines followed by reduction or, alternately from the reduction of hydrazones prepared from carbonyl precursors.

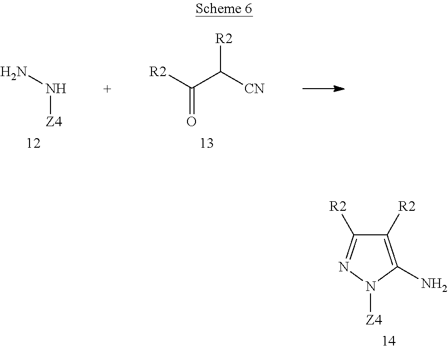

Another preferred method for constructing Z4-substituted pyrazoles is illustrated by the general preparation of pyrazole acids 19 and 20. (Scheme 7), aspects of general acid A-CO₂H 7 (Scheme 3). As indicated in Scheme 7, pyrazole 5-carboxylic esters 17 and 18 can be prepared by the alkylation of pyrazole ester 16 with Z4-X 15, wherein X represents a leaving group on a Z4 moiety such as a halide, triflate, or other sulfonate. Preferred conditions for the alkylation of pyrazole 16 include the use of strong bases such as sodium hydride, potassium tert-butoxide and the like in polar aprotic solovents such as dimethylsulfoxide, dimethylformamide or tetrahydrofuran. Z4-substituted pyrazoles 17 and 18 are isomers of one another and can both be prepared in the same reactions vessel and separated by purification methods familiar to those skilled in the art. The esters 17 and 18 in turn can be converted to acids 19 and 20 using conditions familiar to those skilled in the art, for example saponification in the case of ethyl esters, hydrogenation in the case of benzyl esters or acidic hydrolysis in the case of tert-butyl esters.

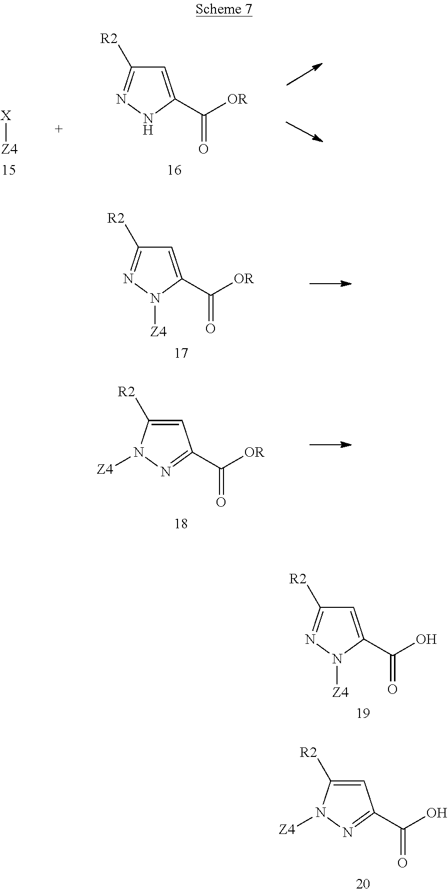

Scheme 8 illustrates the preparation of pyrazole amine 25, a further example of general amine A-NH₂ 6. Acid-catalyzed condensation of R2-substituted hydrazine 21 with 1,1,3,3-tetramethoxypropane 22 provides R2-substituted pyrazole 23. Those skilled in the art will further recognize that R2-substituted pyrazole 23 can also be prepared by direct alkylation of pyrazole. Pyrazole 23 can be regioselectively nitrated to provide nitro-pyrazole 24 by standard conditions familiar to those skilled in the art. Finally, hydrogenation of nitro-pyrazole 24 employing a hydrogenation catalyst, such as palladium or nickel provides pyrazole amine 25, an example of general amine A-NH₂ 6.

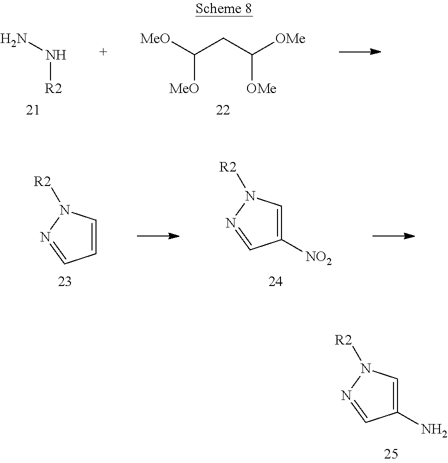

Additional pyrazoles useful for the synthesis of compounds of formula 1 can be preprared as described in Scheme 9. Thus, keto-ester 26 can be reacted with N,N-dimethylformamide dimethyl acetal to provide 27. Reaction of 27 with either 21 or 28 (wherein P is an acid-labile protecting group) in the presence of acid provides 29 or 30. In practice, both 29 and 30 can be obtained from the same reaction and can be separated by standard chromatographic conditions. In turn, esters 29 and 30 can be converted to acids 31 and 32 respectively as described in Scheme 7.

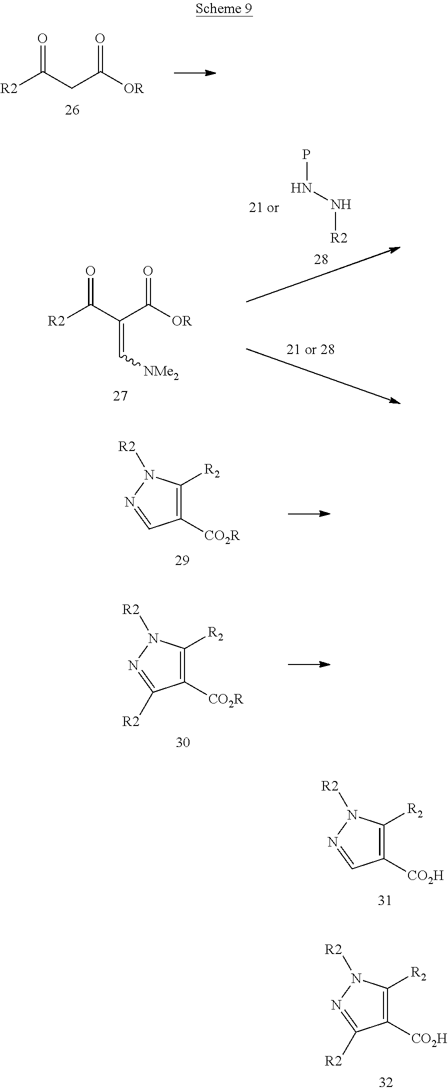

In a manner similar to Scheme 9, NH-pyrazole 34 can be prepared by reaction of acrylate 33 with hydrazine (Scheme 10). Alkylation of 34 with R2-X 35 as described above for Scheme 7 provides mixtures of pyrazole esters 36 and r which are separable by standard chromatographic techniques. Further conversion of esters 36 and 37 to acids 38 and 39 can be accomplished as described in Scheme 7.

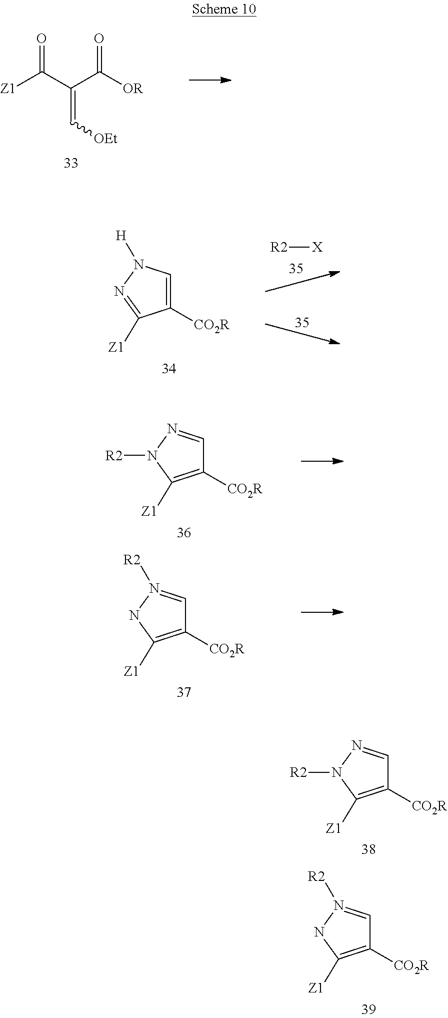

General amines 6 containing an isoxazole ring can be prepared as described in Scheme 11. Thus, by analogy to Scheme 6, reaction of keto-nitrile 9 with hydroxylamine can provide both the 5-aminoisoxazole 40 and 3-aminoisoxazole 41. Preferred conditions for the formation of 5-aminoisoxazole 40 include the treatment of 9 with hydroxylamine in the presence of aqueous sodium hydroxide, optionally in the presence of an alcoholic co-solvent at a temperature between 0 and 100° C. Preferred conditions for the formation of 3-aminoisoxazole 41 include the treatment of 9 with hydroxylamine hydrochloride in a polar solvent such as water, an alcohol, dioxane or a mixture thereof at a temperature between 0 and 100° C.

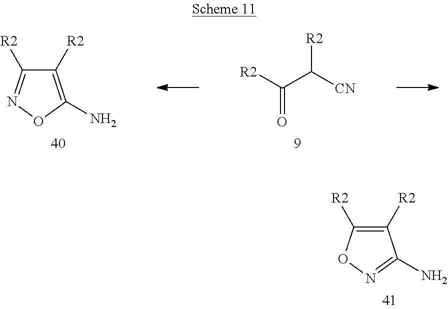

Amines 2 useful for the invention can be synthesized according to methods commonly known to those skilled in the art. Amines of general formula 2 contain three rings and can be prepared by the stepwise union of three monocyclic subunits as illustrated in the following non-limiting Schemes. Scheme 12 illustrates one mode of assembly in which an E-containing subunit 42 is combined with the central pyridine ring 43 to provide the bicyclic intermediate 44. In one aspect this general Scheme, the “M” moiety of 42 represents a hydrogen atom of a heteroatom on the X linker that participates in a nucleophilic aromatic substitution reaction with monocycle 43. Such reactions may be facilitated by the presence of bases (for example, potassium tert-butoxide), thus M may also represent a suitable counterion (for example potassium, sodium, lithium, or cesium) within an alkoxide, sulfide or amide moiety. Alternately, the “M” group can represent a metallic species (for example, copper, boron, tin, zirconium, aluminum, magnesium, lithium, silicon, etc.) on a carbon atom of the X moiety that can undergo a transition-metal-mediated coupling with monocycle 43.

The “Y” group of monocyclic species 42 is an amine or an amine surrogate, such as an amine masked by a protecting group (“P” in formula 45), a nitro group, or a carboxy acid or ester that can be used to prepare an amine via known rearrangement. Examples of suitable protecting groups “P” include but are not limited to tert-butoxycarbonyl (Boc), benzyloxycarbonyl (Cbz), and acetamide. In the instances wherein the “Y”-group of intermediate 42 is not an amine, the products of Scheme 11 will be amine surrogates such as 45 or 46 that can be converted to amine 2 by a deprotection, reduction or rearrangement (for example, Curtius rearrangement) familiar to those skilled in the art.

In these instances, the “LG” of monocycle 43 represents a moiety that can either be directly displaced in a nucleophilic substitution reaction (with or without additional activation) or can participate in a transition-mediated union with fragment 42. The W group of monocycle 43 or bicycle 44 represents a moiety that allows the attachment of the pyrazole. In one aspect, the “W” group represents a halogen atom that will participate in a transition-metal-mediated coupling with a pre-formed heterocyclic reagent (for example a boronic acid or ester, or heteroaryl stannane) to give rise to amine 2. In another aspect, the “W” group of 43 and 44 represents a functional group that can be converted to a five-membered heterocycle by an annulation reaction. Non-limiting examples of such processes would include the conversion of a cyano, formyl, carboxy, acetyl, or alkynyl moiety into a pyrazole moiety. It will be understood by those skilled in the art that such annulations may in fact be reaction sequences and that the reaction arrows in Scheme 11 may represent either a single reaction or a reaction sequence. Additionally, the “W” group of 44 may represent a leaving group (halogen or triflate) that can be displaced by a nucleophilic nitrogen atom of a pyrazole ring.

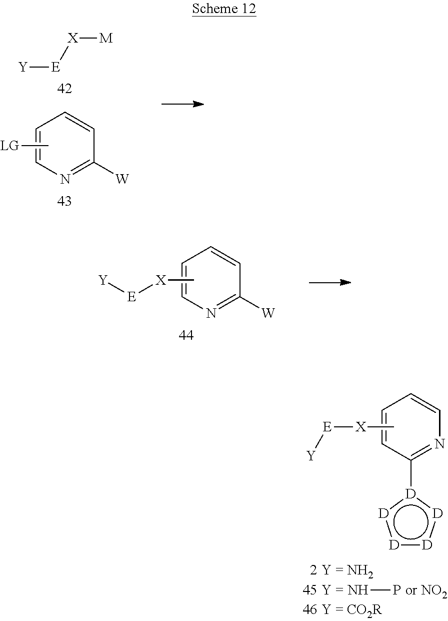

Some non-limiting examples of general Scheme 12 are illustrated in the Schemes below. Scheme 13 illustrates the preparation of pyrazole 51, an example of general amine 2. In Scheme 13, commercially available 3-fluoro-4-aminophenol (47) is reacted with potassium tert-butoxide and 2,4-dichloropyridine 48 to provide chloropyridine 49. The preferred solvent for this transformation is dimethylacetamide at a temperature between 80 and 100° C. Subsequent union of chloropyridine 49 with the commercially available pyrazole-4-boronic acid pinacol ester 50 in the presence of a palladium catalyst, preferably palladium tetrakis(triphenylphosphine), provides amine 51.

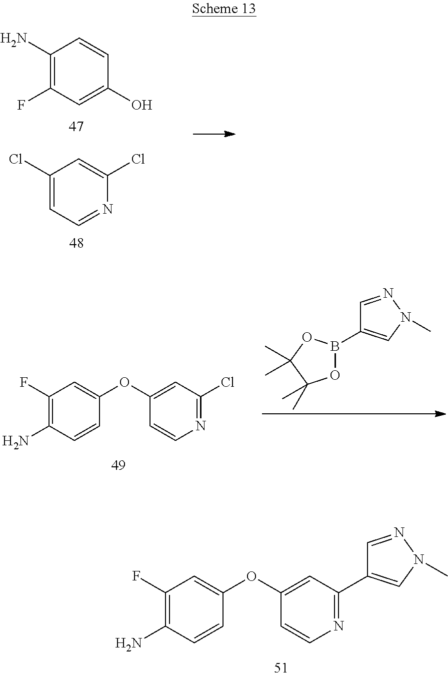

Scheme 14 illustrates a non-limiting examples of Scheme 12 wherein the “W” group is a leaving group for nucleophilic aromatic substitution. Thus, amine 53, an example of general amine 2, can be prepared from general intermediate 49 by reaction with pyrazole (52). Preferred conditions include the use of polar aprotic solvents such as 1-methyl-2-pyrrolidinone, dimethylacetamide, or dimethylsulfoxide in the presence of non-nucleophilic bases such as potassium carbonate, sodium hydride, 1,8-diaza-bicyclo[5.4.0]undec-7-ene (DBU), and the like. Preferred temperatures are from ambient temperature up to about 250° C. and may optionally include the use of microwave irradiation or sonication.

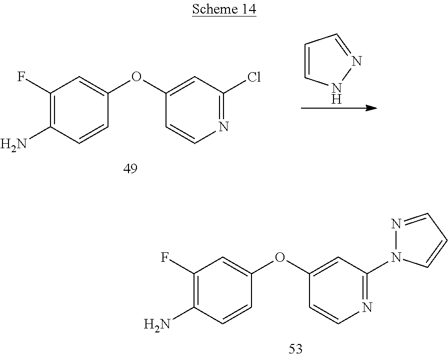

Scheme 15 illustrates the preparation of amine 54, a non-limiting example of a general amine of formula 2 by way of an annulation sequence according to general Scheme 12. Conversion of chloropyridine 49 into alkyne 53 can be accomplished by Sonogashira cross-coupling with trimethylsilylacetylene, followed by aqueous hydrolysis of the trimethylsilyl group, conditions familiar to those skilled in the art. Further reaction of alkyne 53 with trimethylsilyl diazomethane at elevated temperature affords the pyrazole amine 54 (see for example, Tsuzuki, et. al, J. Med. Chem., 2004, (47), 2097).

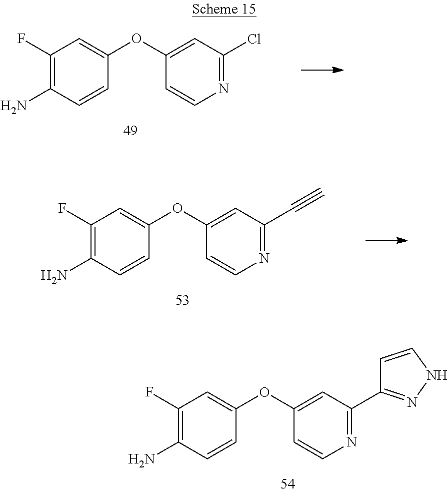

Additional preferred synthetic methods for the preparation of compounds of formula 1 are found in the following examples.

### Section 4. Examples

General Method A: To a solution of the starting pyrazole amine (1 eq) in EtOAc were added 2,2,2-trichloroethylchloroformate (1.1 eq) and saturated NaHCO₃ (2-3 eq) at 0° C. After stirring for 3 h at RT, the layers were separated and the aqueous layer extracted with EtOAc. The combined organic extracts were washed with brine, dried (Na₂SO₄) and concentrated under vacuum to yield the crude TROC carbamate of the pyrazole amine.

To the TROC carbamate (1 eq) in DMSO were added diisopropylethylamine (2 eq), the appropriate amine (2 eq) and the mixture was stirred at 60° C. for 16 h or until all the starting carbamate was consumed. Water was added to the mixture and the product was extracted with EtOAc (2×25 mL). The combined organic extracts were washed with brine solution, dried (Na₂SO₄) and concentrated to yield crude product, which was purified by column chromatography to yield the target compound.

General Method B: To a suspension of the amine (usually 0.67 mmol) in EtOAc (2 mL) was added aqueous 1N NaOH. The reaction mixture was cooled to 0° C. and treated with isopropenyl chloroformate (0.1 mL, 0.94 mmol) over 30 sec. The reaction mixture was stirred for 15 min at 0° C. and 1 h at RT. The reaction was poured into THF-EtOAc (1:1; 40 mL) and washed with H₂O (2×10 mL) and brine (2×10 mL). The organics were dried (Na₂SO₄), concentrated and the residue purified via column chromatography or recrystallization to provide the target (prop-1-en-2-yl)carbamate. To the carbamate (usually 0.26 mmol) was added the appropriate amine (usually 0.26 mmol) in THF (2 mL) and 1-methylpyrrolidine (catalytic amount) and the reaction mixture was sitrred at 60° C. for 18 h. The mixture was diluted with CH₂Cl₂ (2 mL) and hexane (0.5 mL) solution, and stirred for 10 min. The resultant solid was filtered and dried.

General Method C: To a stirring solution of the carboxylic acid (0.24 mmol) and TEA (1.2 mmol) in 1,4-dioxane (4.5 mL) at RT was added DPPA (0.29 mmol). After stirring for 0.5 h at RT, the appropriate amine (0.71 mmol) was added and the reaction was stirred with heating at 100° C. for 2 h. The reaction was cooled to RT, diluted with brine (15 mL) and extracted with EtOAc (3×30 mL). The combined organic layers were dried (MgSO₄) and concentrated. The residue was purified by chromatography to afford the target compound.

General Method D: To a stirring suspension of amine (3.2 mmol, 1.0 eq) in THF (6 ml) at −78° C. was added 1.0M LiHMDS/THF (6.4 mmol, 2.00 eq). After 30 min at −78° C., the resulting solution was treated with isopropenyl chloroformate (3.2 mmol, 1.0 eq). After another 30 min at −78° C., the completed reaction was diluted with 3M HCl, warmed to RT and extracted with EtOAc (2×). The combined organics were washed with H₂O (1×), satd. NaHCO₃ (1×), and brine (1×), dried (MgSO₄), filtered and concentrated in vacuo to afford the target prop-1-en-2-yl carbamate which was used as is, purified by silica gel chromatography or recrystallized.

To the carbamate (usually 0.26 mmol) was added the appropriate amine (usually 0.26 mmol) in THF (2 mL) and 1-methylpyrrolidine (catalytic amount) and the reaction was stirred at 60° C. for 18 h. The mixture was diluted with CH₂Cl₂ (2 mL) and hexane (0.5 mL) solution, and stirred for 10 min. The resultant solid was filtered and dried and the resulting solid converted to the amine hydrochloride salt by treatment with 0.1 N HCl solution and lyophilization or purified via column chromatograhpy.

General Method E: To a stirring solution of amine (2 mmol, 1.00 eq) and pyridine (4 mmol, 2.00 eq) in CH₂Cl₂ (18 ml) at RT was added isopropenyl chloroformate (1.87 mmol, 1.05 eq). After 4 hours the reaction was washed with 3M HCl (1×), satd. NaHCO₃ (1×), dried (Na₂SO₄), filtered and evaporated to afford the target prop-1-en-2-yl carbamate. The material was used as is in the next reaction.

To the carbamate (usually 0.26 mmol) was added the appropriate amine (usually 0.26 mmol) in THF (2 mL) and 1-methylpyrrolidine (catalytic amount) and the reaction was stirred at 60° C. for 18 h. The mixture was diluted with CH₂Cl₂ (2 mL) and hexane (0.5 mL) solution, and stirred for 10 min. The resultant solid was filtered and dried.

General Method F: To a solution of amine (6.53 mmol) in ethyl acetate (20 mL) at RT was added a solution of sodium bicarbonate (11.90 mmol) in water (20 mL) and isopropenyl chloroformate (9.79 mmol). The resultant mixture was stirred for 3 h at RT. The organic layer was separated. The aqueous layer was extracted once with ethyl acetate. The combined organic extracts were washed with brine, dried (MgSO₄) and concentrated in vacuo. The residue was used without further purification or purified via recrystallization or chromatography to provide the corresponding prop-1-en-2-yl carbamate.

### Example A1

A suspension of 3-fluoro-4-aminophenol (8.0 g, 63.0 mmol) in dimethylacetamide (80 mL) was de-gassed in vacuo and treated with potassium tert-butoxide (7.3 g, 65 mmol). The resultant mixture was stirred at RT for 30 min. 2,4-Dichloropyridine (8 g, 54 mmol) was added and the mixture was heated to 80° C. for 12 h. The solvent was removed under reduced pressure to give a residue which was partitioned between water and EtOAc (3×100 mL). The organic layers were washed with saturated brine, dried (MgSO₄), concentrated in vacuo and purified by silica gel column chromatography to give 4-(2-chloro-pyridin-4-yloxy)-2-fluoro-phenylamine (11 g, 86% yield). ¹H NMR (300 MHz, DMSO-d₆), δ 8.24 (d, J=5.7 Hz, 1H), 7.00 (dd, J=9.0, 2.7 Hz, 1H), 6.89-6.73 (m, 4H), 5.21 (br s, 2H); MS (ESI) m/z: 239.2 (M+H+).

A solution of 4-(2-chloropyridin-4-yloxy)-2-fluorobenzenamine (3 g, 12.6 mmol), 1-methyl-3-(4,4,5,5-tetramethyl-[1,3,2]dioxaborolan-2-yl)-1H-pyrazole (5.2 g, 25.2 mmol), and Na₂CO₃ (2.7 g, 25.2 mmol) in DME (18 mL) and water (6 mL) was sparged with nitrogen for 20 min. Pd(PPh₃)₄ (729 mg, 0.63 mmol) was added and the resulting mixture was heated to 100° C. for 16 h. The solvent was removed under reduced pressure and the crude product was suspended in water and extracted with EtOAc. The organic layer was washed with brine, dried (Na₂SO₄), filtered, concentrated in vacuo and purified via silica gel chromatography to give 2-fluoro-4-(2-(1-methyl-1H-pyrazol-4-yl)pyridin-4-yloxy)benzenamine (2 g, 56% yield). ¹H NMR (300 MHz, DMSO-d₆): δ 8.31 (d, J=5.7 Hz, 1H), 8.21 (s, 1H), 7.92 (s, 1H), 7.12 (d, J=2.4 Hz, 1H), 6.96 (m, 1 H), 6.85-6.72 (m, 2H), 6.56 (m, 1H), 5.15 (s, 2H), 3.84 (s, 3H); MS (ESI) m/z: 285.0 (M+H⁺).

### Example A2

4-amino-phenol (8.9 g, 81.6 mmol) and potassium tert-butoxide (10.7 g, 95.2 mmol) were suspended in DMF (100 mL) and stirred at RT for 30 min. 2,4-Dichloro-pyridine (10 g, 68 mmol) was added and the resulting mixture was heated to 90° C. for 3 h. The solvent was removed under vacuum and the residue was extracted with DCM (2×100 mL). The combined organics were dried (MgSO₄), concentrated in vacuo and purified by silica gel chromatography to afford 4-(2-chloro-pyridin-4-yloxy)-phenylamine (9.0 g, 60% yield). ¹H NMR (DMSO-d₆): δ 8.21 (d, J=5.6 Hz, 1H), 6.85-6.82 (m, 4H), 6.61 (d, J=6.6 Hz, 2H), 5.17 (s, 2H); MS (ESI) m/z: 221 (M+H⁺).

4-(2-Chloro-pyridin-4-yloxy)-phenylamine (0.7 g, 3.2 mmol), 1-methyl-4-(4,4,5,5-tetramethyl)-[1,3,2]dioxaborolan-2-yl)-4H-pyrazole (1.0 g, 4.8 mmol), Cs₂CO₃ (4.0 g, 12.3 mmol) and Pd(PPh₃)₄ (0.45 g, 0.4 mmol) were combined in a mixture of DMF and water (3; 1.20 mL). The reaction mixture was degassed, blanketed with argon and heated to 90° C. overnight. The reaction mixture was diluted with water and extracted with EtOAc (3×50 mL). The combined organics were washed with saturated brine, dried (MgSO₄), concentrated in vacuo and purified by silica gel chromatography to provide 4-(2-(1-methyl-1H-pyrazol-4-yl)pyridin-4-yloxy)benzenamine (0.7 g, 74% yield). ¹H NMR (300 MHz, DMSO-d₆), δ 8.29 (d, J=5.7 Hz, 1H), 8.19 (s, 1H), 7.90 (s, 1H), 7.10 (d, J=2.4 Hz, 1H), 6.83 (d, J=8.7 Hz, 2H), 6.62 (d, J=8.7 Hz, 2H), 6.52 (dd, J=2.4, 5.7 Hz, 1H), 5.10 (s, 2H), 3.84 (s, 3H); MS (ESI) m/z: 267.3 (M+H⁺).

### Example A3

1,2,3-Trifluoro-4-nitro-benzene (30 g, 0.17 mol), benzyl alcohol (18.4 g, 0.17 mol) and K₂CO₃ (35 g, 0.25 mol) were combined in DMF (300 mL) and were stirred at RT for 8 h. Water (300 mL) was added, and the mixture was extracted with EtOAc (3×500 mL). The combined organic layers were washed with brine, dried (MgSO₄), concentrated in vacuo and purified by column chromatography on silica gel to give 1-benzyloxy-2,3-difluoro-4-nitro-benzene (16 g, 36% yield). ¹HNMR (400 MHz, DMSO-d₆): δ 8.06 (m, 1H), 7.49-7.30 (m, 6H), 5.37 (s, 2H).

A solution of 1-benzyloxy-2,3-difluoro-4-nitro-benzene (14 g, 52.8 mmol) in MeOH (200 mL) was stirred with Pd/C (10%, 1.4 g, 1.3 mmol) under a hydrogen atmosphere (30 psi) for 2 h. The catalyst was removed by filtration, and the filtrate was concentrated in vacuo to afford 4-amino-2,3-difluorophenol (7 g, 92.1% yield). ¹H NMR (400 MHz, DMSO-d₆): δ 9.05 (s, 1H), 6.45 (t, J=8.8 Hz, 1H), 6.34 (t, J=9.2 Hz, 1H), 4.67 (s, 2H); MS (ESI) m/z: 146.1 [M+H]⁺.

4-amino-2,3-difluorophenol (6 g, 41.4 mmol) and potassium tert-butoxide (4.9 g, 43.5 mmol) were suspended in DMAc (200 mL) and stirred at RT for 30 min under Ar atmosphere. 2,4-Dichloropyridine (6.1 g, 41.4 mmol) was added, and the resulting mixture was heated at 70° C. for 8 h. The reaction mixture was filtered, concentrated in vacuo and purified by silica gel chromatography to afford 4-(2-chloro-pyridin-4-yloxy)-2,3-difluoro-phenylamine (7 g, 66% yield). ¹H NMR (400 MHz, DMSO-d₆): δ 8.27 (d, J=6.0 Hz, 1H), 7.05 (s, 1H), 6.95 (m, 1H), 6.92 (m, 1H), 6.62 (m, 1H), 5.60 (s, 2H); MS (ESI) m/z: 257.1 [M+H]⁺.

Nitrogen was bubbled though a solution of 4-(2-chloro-pyridin-4-yloxy)-2,3-difluoro-phenylamine (2 g, 7.8 mmol), 1-methyl-4-(4,4,5,5-tetramethyl-[1,3,2]dioxaborolan-2-yl)-1H-pyrazole (1.6 g, 7.8 mmol) and Na₂CO₃ (1.65 g, 15.6 mmol) in DME (12 mL) and H₂O (4 mL) for 20 min. Pd(PPh₃)₄ (450 mg, 0.4 mmol), was added and then resulting mixture was degassed in vacuo, blanketed with nitrogen and heated to 70° C. for 16 h. The reaction was concentrated to dryness under reduced pressure. The crude product was suspended in water and extracted with EtOAc (3×10 mL). The organic layer was washed with brine, dried (Na₂SO₄), concentrated in vacuo and purified by silica gel chromatography to give 2,3-difluoro-4-[2-(1-methyl-1H-pyrazol-4-yl)-pyridin-4-yloxy]-phenylamine (1.3 g, 55% yield). ¹H NMR (400 MHz, DMSO-d₆), δ 8.40 (d, J=6.0 Hz, 1H), 8.32 (s, 1H), 8.02 (s, 1H), 7.26 (s, 1H), 6.96 (t, J=8.8 Hz, 1H), 6.71-6.68 (m, 2H), 5.62 (s, 2H), 3.92 (s, 3H); MS (ESI) m/z: 303.2 [M+H]⁺.

### Example A4

A solution of 1,3-difluoro-2-methyl-benzene (15 g, 0.12 mol) in conc. H₂SO₄ (100 mL) was treated drop wise with 65% HNO₃ (11.4 g, 0.12 mol) at −10° C. and the resultant mixture was stirred for about 30 min. The mixture was poured into ice-water and extracted with ethyl acetate (3×200 mL). The combined organic layers were washed with brine, dried (Na₂SO₄) and concentrated in vacuo to give 1,3-difluoro-2-methyl-4-nitro-benzene (16 g, 78% yield)¹H NMR (400 MHz, CDCl₃): δ 7.80 (m, 1H), 6.95 (m, 1H), 2.30 (s, 3H).

1,3-Difluoro-2-methyl-4-nitro-benzene (16 g, 0.092 mol), benzyl alcohol (10 g, 0.092 mol) and K₂CO₃ (25.3 g, 0.18 mol), were combined in DMF (300 mL) and heated to 100° C. overnight. The mixture was poured into water and extracted with ethyl acetate (3×200 mL). The combined organic layers were washed with brine, dried (Na₂SO₄), concentrated in vacuo and purified by silica gel chromatography to give 1-benzyloxy-3-fluoro-2-methyl-4-nitro-benzene (8 g, 33% yield). ¹H NMR (400 MHz, DMSO-d₆): δ 8.04 (t, J=8.8 Hz, 1H), 7.30-7.46 (m, 5H), 7.08 (d, J=9.2 Hz, 1H), 5.28 (s, 2H), 2.13 (s, 3H).

Using a procedure analogous to Example A3,1-benzyloxy-3-fluoro-2-methyl-4-nitro-benzene (8 g, 0.031 mol) was hydrogenated to give 4-amino-3-fluoro-2-methyl-phenol (4.2 g, 96% yield). ¹H NMR (300 MHz, DMSO-d₆): δ 8.61 (s, 1H), 6.36 (m, 2H), 4.28 (s, 2H), 1.96 (s, 3H); MS (ESI) m/z: 142.1 [M+H]⁺.

Potassium tert-butoxide (3.5 g, 31 mmol) was added to a solution of 4-amino-3-fluoro-2-methyl-phenol (4.2 g, 30 mmol) in dimethylacetamide. The mixture was stirred at RT for 30 min. A solution of 2,4-dichloropyridine (4.38 g, 30 mmol) in dimethylacetamide was added and the mixture was heated at 100° C. overnight. The reaction mixture was concentrated in vacuo and the residue was dissolved in ethyl acetate (200 mL) and filtered through silica gel. The filter cake was washed with ethyl acetate and the combined filtrates were concentrated in vacuo and purified by silica gel chromatography to give 4-(2-chloro-pyridin-4-yloxy)-2-fluoro-3-methyl-phenylamine (3.2 g, 42% yield). ¹H NMR (400 MHz, DMSO-d₆): δ 8.21 (d, J=6.4 Hz, 1H), 6.84 (d, J=2.0 Hz, 1H), 6.81 (dd, J=5.6, 2.4 Hz, 1H), 6.67-6.65 (m, 2H), 5.13 (s, 2H), 1.91 (s, 3H); MS (ESI): m/z 253.2 [M+H]⁺.

Using a procedure analogous to Example A3,4-(2-chloro-pyridin-4-yloxy)-2-fluoro-3-methyl-phenylamine (1.0 g, 3.3 mmol), 1-methyl-4-(4,4,5,5-tetramethyl-[1,3,2]dioxaborolan-2-yl)-1H-pyrazole (1 g, 4.8 mmol), Na₂CO₃ (0.84 g, 6.6 mmol) and Pd(PPh₃)₄ (0.25 g, 0.2 mmol) were combined to give 2-fluoro-3-methyl-4-[2-(1-methyl-1H-pyrazol-4-yl)-pyridin-4-yloxy]-phenylamine (0.74 g, 75% yield). ¹H NMR (400 MHz, DMSO-d₆): δ 8.27 (d, J=6.4 Hz, 1H), 8.18 (s, 1H), 7.90 (s, 1H), 7.07 (s, 1 H), 6.68-6.61 (m, 2H), 6.45 (dd, J=5.6, 2.4 Hz, 1H), 5.06 (s, 2H), 3.82 (s, 3H), 1.95 (s, 3H); MS (ESI) m/z: 299.2 [M+H]⁺.

### Example B1

To an aqueous solution of sodium hydroxide solution (40.00 g, 1 mol, in 200 ml of water) was added hydroxylamine hydrochloride (24.00 g, 346 mmol) and pivaloylacetonitrile (40.00 g, 320 mmol). The resulting solution was stirred at 50° C. for 3 hrs. The reaction mixture cooled and the resultant white crystalline solid filtered, washed with water and dried to provide 3-t-butylisoxazol-5-amine as a white crystalline solid (34 g, yield 76% yield). ¹H NMR (DMSO-d₆) δ 6.41 (brs, 2H), 4.85 (s, 1H), 1.18 (s, 9H): LC-MS (ES, m/z, M+H) 141.3.

### Example B2

Methyl hydrazine and 4,4-dimethyl-3-oxopentanenitrile were combined according to literature procedures to yield 3-t-butyl-1-methyl-1H-pyrazol-5-amine. See WO 2006/071940.

### Example B3

t-Butylhydrazine and 1,1,3,3-tetramethoxypropane were combined according to literature procedures to yield 1-t-butyl-1H-pyrazol-4-amine. See Ger. Offen., DE3332270, 21 Mar. 1985.

### Example B4

To a suspension of KCN (1.90 g, 29.1 mmol) in MeOH (35 mL) was added dropwise 3-bromo-1,1,1-trifluoropropan-2-one oxime (5.00 g, 24.3 mmol) in MeOH (72 mL) at RT. The reaction mixture was stirred at RT for 3 hours. The solution was concentrated in vacuo, the residue was dissolved in EtOAc and stirred at RT. The solid was filtered and the filtrate was evaporated to obtain the crude product. The crude product was purified by silica gel column chromatography (EtOAc/hexanes) to obtain 3-(trifluoromethyl)isoxazol-5-amine (1.38 g, 37% yield). MS (ESI) m/z: 153.0 (M+H⁺).

### Example B5

Using a procedure analogous to Example B6, ethyl 1-tert-butyl-5-(trifluoromethyl)-1H-pyrazole-4-carboxylate (750 mg, 2.84 mmol) was converted to 1-tert-butyl-5-(trifluoromethyl)-1H-pyrazole-4-carboxylic acid (646 mg, 94% yield) using lithium hydroxide hydrate (357 mg, 8.51 mmol). ¹H NMR (300 MHz, DMSO-d₆), δ 1.63 (s, 9H), 7.92 (s, 1H); MS (ESI) m/z: 259.0 (M+Na⁺).

### Example B6

In ethanol (10 mL) was placed the tert-butylhydrazine hydrochloride (1.35 g, 10.8 mmol) and ethyl 2-((dimethylamino)methylene)-3-oxobutanoate (2.00 g, 10.8 mmol). The mixture warmed to reflux and stirred for 2 hrs, then cooled to RT and stirred overnight. The mixture was evaporated at reduced pressure to give an oil which was dissolved in ether (25 mL) and washed successively with water (25 mL), saturated sodium bicarbonate (25 mL) and brine (25 mL), dried (Na₂SO₄), evaporated at reduced pressure and purified by chromatography (S1-25 column, ethyl acetate/hexanes) to give ethyl 1-tert-butyl-5-methyl-1H-pyrazole-4-carboxylate (1.48 g, 65% yield) as an oil. MS (ESI) m/z: 211.0 (M+H⁺).

In a mixture of ethanol:water:dioxane (1:1:1, 21 mL) was placed ethyl 1-tert-butyl-5-methyl-1H-pyrazole-4-carboxylate (1.48 g, 7.04 mmol) and lithium hydroxide hydrate (886 mg, 21.12 mmol). The reaction was stirred at 40° C. for 3 hrs and then at RT overnight. The reaction was diluted with water (25 mL) and ether (25 mL). The ether layer was discarded and the aqueous phase made acidic (pH˜=4) with 1N HCl. The acidic phase was then extracted with ethyl acetate (2×25 mL) and the combined ethyl acetate layers were washed with brine, dried (Na₂SO₄), and evaporated at reduced pressure to give 1-tert-butyl-5-methyl-1H-pyrazole-4-carboxylic acid as a white solid (1.12 g, 87% yield). ¹H NMR (300 MHz, DMSO-d₆): δ 1.56 (s, 9H), 2.67 (s, 3H), 7.65 (s, 1H), 12.13 (s, 1H); MS (ESI) m/z: 183.0 (M+H⁺).

### Example B7

A solution of nBuLi in hexanes (242 mL, 387 mmol) was added to a −78° C. solution of diisopropylamine (39.1 g, 387 mmol) in anhydrous THF (300 mL) and the resultant mixture was stirred for 30 min at −78° C. A solution of ethyl cyclopentanecarboxylate (50 g, 352 mmol) in anhydrous THF (150 mL) was added dropwise into the mixture and the reaction mixture was stirred at −78° C. for 1 h. Iodomethane (79.2 g, 558 mmol) was added dropwise and the resulting mixture was warmed to RT and stirred overnight. The mixture was poured into water and extracted with ethyl ether. The combined extracts were washed with brine, dried (MgSO₄) and concentrated in vacuo to give ethyl 1-methylcyclopentanecarboxylate (47 g, 85%). ¹H NMR (300 MHz, DMSO-d₆): δ 4.03 (q, J=7.2 Hz, 2H), 1.37-2.03 (m, 8H), 1.15-1.12 (m, 6H).

Ethyl 1-methylcyclopentanecarboxylate (47 g, 301 mmol), acetonitrile (14.5 g, 363 mmol), NaH (18 g, 450 mmol), NaOH (6.8 g, 170 mmol) and hydroxylamine hydrochloride (4 g, 57 mmol) were sequentially combined by a procedure analogous to Example B10 to provide 3-(1-methylcyclopentyl)isoxazol-5-amine (7 g, 70% yield over 2 steps). ¹H NMR (400 MHz, DMSO-d₆): δ 6.41 (s, 2H), 4.81 (s, 1H), 1.91-1.86 (m, 2 H), 1.67-1.48 (m, 6H), 1.19 (s, 3H); MS (ESI) m/z: 167.1 (M+H⁺).

### Example B8

Sodium metal (13.8 g, 0.5 mol) was added portionwise to ice-cold anhydrous EtOH (700 mL). After complete dissolution of the Na, a mixture of 3,3-dimethylbutan-2-one (50 g, 0.5 mol) and oxalic acid diethyl ester (77 ml, 0.5 mol) was added drop-wise. The reaction mixture was stirred in ice-salt bath until TLC indicated completion of the reaction. Acetic acid (38.1 ml, 0.5 mol) was added and the mixture was stirred at RT for 30 min. The reaction mixture was cooled in an ice-salt bath and treated with hydrazine hydrate (29.4 g, 0.5 mol). After complete addition, the mixture was warmed to RT and stirred until judged complete by TLC. The reaction mixture was concentrated under reduced pressure and re-dissolved in EtOAc. The EtOAc solution was washed with NaHCO₃, brine and water, dried (MgSO₄) and concentrated in vacuo. The resultant solid was washed with cold petroleum ether to give ethyl 3-tert-butyl-1H-pyrazole-5-carboxylate (49 g, 50% yield over two steps) as a white solid. ¹H NMR (400 MHz, CDCl₃): δ 6.65 (s, 1H), 4.38 (q, J=6.8 Hz, 2H), 1.39 (t, J=6.8 Hz, 3H), 1.35 (s, 1H); MS (ESI) m/z: 197.2 (M+H⁺).

Potassium t-butoxide (2.6 g, 23 mmol) was dissolved in DMSO (10 mL) and to this solution was added ethyl 3-tert-butyl-1H-pyrazole-5-carboxylate (4.5 g, 23 mmol) in small portions and stirred under Ar for 15 min. To this solution was added t-butyl-bromoacetate (5.4 g, 28 mmol) slowly at 0° C. with stirring for 45 min at RT. Sat. NH₄Cl solution was added and product was extracted with ethyl acetate (3×50 mL). The combined organic layers were washed with brine, dried (Na₂SO₄) and concentrated to afford (7.0 g) coupled product as a pasty mass. The above pasty mass was dissolved in TFA (10 mL) and stirred for 3 h at RT. Solvents were removed, water (100 mL) was added and product was extracted with DCM (3×50 ml). The combined organic extracts were washed with brine solution, dried (Na₂SO₄) and concentrated to yield 2-(3-tert-butyl-5-(ethoxycarbonyl)-1H-pyrazol-1-yl)acetic acid (5.8 gm, 100%) as a pasty mass. ¹H NMR (400 MHz, Acetone-d₆): δ 6.78 (s, 1H), 5.25 (s, 2H), 4.30 (q, J=7.2 Hz, 2H), 1.35-1.30 (m, 12H); MS (ESI) m/z: 255.2 (M+H⁺).

To a solution of acid (0.41 g, 1.6 mmol) in DMF (5 mL) was added PyBop (0.84 g, 1.6 mmol), DIPEA (0.42 g, 3.2 mmol) and dimethylamine hydrochloride (0.26 g, 3.2 mmol). After stirring the mixture for 1 h at RT, water (50 mL) was added, and the product was extracted with ethyl acetate (2×30 ml). The combined organic layers were washed with 3M HCl solution (1×30 mL), dried (Na₂SO₄) and concentrated to afford crude product which was purified by chromatography (EtOAc/DCM) to afford ethyl 3-tert-butyl-1-(2-(dimethylamino)-2-oxoethyl)-1H-pyrazole-5-carboxylate (0.25 g, 55%) as a thick paste. ¹H NMR (400 MHz, Acetone-d₆): δ 6.73 (s, 1H), 5.35 (s, 2H), 4.27 (q, J=7.2 Hz, 2H), 3.15 (s, 3H), 2.90 (s, 3H), 1.33-1.28 (m, 12H); MS (ESI) m/z: 282.3 (M+H⁺).

To a solution of ethyl 3-tert-butyl-1-(2-(dimethylamino)-2-oxoethyl)-1H-pyrazole-5-carboxylate (1.16 g, 4 mmol) in THF (10 mL) was added 1M borane/THF (12 ml, 12 mmol) at 0° C. under Ar and stirring continued for 12 h at 60° C. The mixture was cooled to 0° C., quenched with 3M HCl solution and heated to 60° C. for 30 min. The mixture was basified with solid NaHCO₃ to pH around 8 and the product was extracted with CHCl₃ (2×30 ml). The combined organics were washed with brine, dried (Na₂SO₄), concentrated in vacuo and purified by silica gel chromatography to provide ethyl 3-tert-butyl-1-(2-(dimethylamino)ethyl)-1H-pyrazole-5-carboxylate as a pasty mass (0.47 g, 43% yield). ¹H NMR (400 MHz, MeOH-d₄): δ 6.73 (s, 1H), 4.66 (t, J=6.8 Hz, 2H), 4.35 (q, J=7.2 Hz, 2H), 2.80 (t, J=7.2 Hz, 2H), 2.34 (s, 6H), 1.38 (t, J=7.2 Hz, 3H), 1.31 (s, 9H); MS (ESI) m/z: 268.2 (M+H⁺).

To a solution of ethyl 3-tert-butyl-1-(2-(dimethylamino)ethyl)-1H-pyrazole-5-carboxylate (0.47 g, 1.8 mmol) in THF (10 mL) was added aqueous LiOH (0.22 g, 5.3 mmol, 5 mL) and the mixture was stirred for 16 h at RT. Solvents were removed, the thick liquid was diluted with water (5 mL) and acidified with 50% aq. acetic acid solution to pH 5-6. The product was extracted with EtOAc (2×50 ml) and the combined organics were washed with brine, dried (Na₂SO₄) and concentrated in vacuo to afford 3-tert-butyl-1-(2-(dimethylamino)ethyl)-1H-pyrazole-5-carboxylic acid as a pasty mass (0.12 g, 29% yield). ¹H NMR (400 MHz, DMSO-d₆): δ 6.56 (s, 1H), 4.66 (t, J=6.0 Hz, 2H), 3.17 (t, J=6.0 Hz, 2H), 2.53 (s, 6H), 1.17 (s, 9H); MS (ESI) m/z: 240.3 (M+H⁺).

### Example B9

NaH (6.8 g, 0.17 mol) was added portionwise to a 0° C. solution of 1H-pyrazole (10 g, 0.15 mol) in DMF (150 mL) and the resulting mixture was stirred at RT for 30 min. 2-Iodopropane (30 mL, 0.3 mol) was added dropwise to the above mixture at 0° C., then the reaction mixture was stirred at RT for 10 h. H₂O was added and the mixture was extracted with ethyl ether (3×100 mL). The combined organic layers were washed with brine, (Na₂SO₄), concentrated in vacuo and the residue distilled under reduced pressure to afford 1-isopropyl-1H-pyrazole (6.6 g, 40% yield). ¹H NMR (400 MHz, DMSO-d₆): δ 7.68 (d, J=1.6 Hz, 1H), 7.38 (d, J=1.2 Hz, 1H), 6.17 (t, J=2.0 Hz, 1H), 4.46 (m, 1H), 1.37 (d, J=6.8 Hz, 6H).

To a solution of 1-isopropyl-1H-pyrazole (5 g, 45.5 mmol) in conc. H₂SO₄ (50 mL) was added KNO₃ (5.0 g, 50 mmol) portionwise at 0° C. After the addition, the resulting mixture was heated to 50° C. for 8 h. The reaction mixture was cooled to RT, poured into ice water, and the mixture was extracted with EtOAc. The combined organics were washed with saturated Na₂CO₃ solution, brine, dried (Na₂SO₄), concentrated in vacuo and purified via column chromatography to provide 1-isopropyl-4-nitro-1H-pyrazole (3.2 g, 46% yield). ¹H NMR (400 MHz, DMSO-d₆): δ 8.99 (s, 1H), 8.32 (s, 1 H), 4.65 (m, 1H), 1.51 (d, J=6.8 Hz, 6H).

A solution of 1-isopropyl-4-nitro-1H-pyrazole (3 g, 19 mmol) in EtOH (30 mL) was stirred under a hydrogen atmosphere for 2 h in the presence of 10% Pd/C (300 mg). The catalyst was removed by filtration and the filtrate was concentrated under reduced pressure to afford 1-isopropyl-1H-pyrazol-4-ylamine (1.8 g, 75% yield). ¹H NMR (400 MHz, DMSO-d₆): δ 6.99 (s, 1H), 6.84 (s, 1H), 4.23 (m, 1H), 3.70 (s, 2H), 1.28 (d, J=6.8 Hz, 6H); MS (ESI) m/z: 126.2 [M+H]⁺.

### Example B10

A solution of ethyl cyclopentanecarboxylate (prepared by esterification of commercially available cyclopentantecarboxylic acid, 30 g, 0.21 mol) and acetonitrile (10.1 g, 0.25 mol) in dry THF (80 mL) was added dropwise to a suspension of NaH (12.5 g, 0.31 mol) in dry THF (80 mL) and the resulting mixture was refluxed overnight. The reaction mixture was concentrated under reduced pressure and partitioned between water and EtOAc. The aqueous layer was separated, adjusted to pH 8 and extracted with EtOAc. The combined extracts were washed with brine, dried (MgSO₄), and concentrated to give 3-cyclopentyl-3-oxopropanenitrile (26 g, 90% yield), which was used in the next step without further purification. ¹H NMR (400 MHz, DMSO-d₆): δ 4.06 (s, 2H), 2.92 (m, 1H), 1.41-1.77 (m, 8H).

Hydroxylamine hydrochloride (6 g, 86 mmol) and 3-cyclopentyl-3-oxopropanenitrile (10 g, 73 mmol) were added to a solution of NaOH (9 g, 225 mmol) in water (100 mL) and the resulting mixture was heated at 50° C. overnight. The precipitate was collected by filtration, washed with water, and dried to give 3-cyclopentylisoxazol-5-amine (6.7 g, 61% yield). ¹H NMR (400 MHz, DMSO-d₆): δ 6.43 (s, 2H), 4.77 (s, 1H), 2.84 (m, 1H), 1.87-1.51 (m, 8H); MS (ESI) m/z: 153.1 (M+H⁺).

### Example B11

A mixture of 1,1,3,3-tetramethoxy-propane (13.6 g, 83 mmol) and 1-cyclopentylhydrazine-2-carboxylic acid tert-butyl ester from Ex B18 (16.6 g, 83 mmol) in water (150 mL) was treated with conc HCl (21 mL, 252 mmol) and the resulting mixture was heated at reflux overnight. The reaction mixture was allowed to cool to RT and was extracted with ether. The extracts were washed with brine, dried over anhydrous MgSO₄ and filtered. The filtrate was concentrated in vacuo to give 1-cyclopentyl-1H-pyrazole (8.0 g, 71% yield). ¹H NMR (400 MHz, CDCl₃): δ 7.52 (s, 1H), 7.43 (s, 1H), 6.24 (s, 1H), 4.68 (m, 1H), 2.20-1.71 (m, 8H); MS (ESI) m/z: 137.1 [M+H⁺]

To a suspension of Na₂CO₃ (13 g, 124 mmol) in DCM (100 mL) was added 1-cyclopentyl-1H-pyrazole (8.35 g, 62 mmol) and Br₂ (3.2 mL, 62.3 mmol). The resulting mixture was stirred at RT overnight. The solids were removed by filtration and the filter cake was washed with DCM. The filtrate was washed with water and brine, was dried over anhydrous MgSO₄, and was concentrated in vacuo to give 4-bromo-1-cyclopentyl-1H-pyrazole (14 g, 93% yield). ¹H NMR (300 MHz, CDCl₃): δ 7.46 (s, 1H), 7.44 (s, 1 H), 4.64 (m, 1H), 2.18-1.67 (m, 8H); MS (ESI) m/z: 215.0 [M+H]⁺.

To a solution of 4-bromo-1-cyclopentyl-1H-pyrazole (9.0 g, 42 mmol) in THF (100 mL) at −78° C. under nitrogen was added a solution of n-BuLi in hexanes (2.5 M, 18.5 mL, 46.2 mmol). The resulting mixture was stirred at −78° C. for 30 min. Dry-ice (solid CO₂) was added at −78° C. and the reaction mixture was allowed to slowly warm to RT overnight. The solvent was removed under reduced pressure. Water was added, and the mixture was acidified (pH 3) by the addition of aq. HCl. The aqueous layer was extracted with EtOAc, and the extracts were washed with brine, dried over MgSO₄, and concentrated in vacuo. The residue was recrystallized (EtOAc-petroleum ether) to provide 1-cyclopentyl-1H-pyrazole-4-carboxylic acid (3.5 g, 47% yield). ¹H NMR (400 MHz, DMSO-d₆): δ 12.50 (br s, 1H), 8.31 (s, 1H), 7.85 (s, 1H), 4.78 (m, 1H), 2.16-1.68 (m, 8H); MS (ESI) m/z: 181.0 [M+H]⁺.

### Example B12

A solution of ethyl trifluoroacetate (14.2 g, 0.1 mol) and anhydrous acetonitrile (5.0 g, 0.12 mol) in THF (100 mL) was added dropwise to a suspension of NaH (60%, 6.0 g, 0.15 mol) in THF (100 mL) at 80° C. The resulting mixture was heated to reflux overnight, and then cooled to RT. The reaction mixture was concentrated in vacuo and the residue was diluted with EtOAc and 10% aq HCl. The organic layer was washed with water and brine, dried (MgSO₄) and concentrated in vacuo to yield crude 4,4,4-trifluoro-3-oxo-butyronitrile (15 g), which was used without further purification.

A solution of methylhydrazine (5.0 g, 60 mmol) and 4,4,4-trifluoro-3-oxo-butyronitrile (9.8 g, 71 mmol) in EtOH (50 mL) was treated with conc. HCl (5 mL) and the resultant mixture was heated to reflux overnight. The solvent was removed in vacuo and the crude product was dissolved in EtOAc washed with saturated aq. Na₂CO₃ solution until the washings were pH 8. The organics were concentrated and purified by prep-HPLC to provide 2-methyl-5-trifluoromethyl-2H-pyrazol-3-ylamine (2.07 g, 21% yield). ¹H NMR (300 MHz, DMSO-d6), δ 5.57 (s, 1H), 5.54 (br s, 2H), 3.55 (s, 3H); MS (ESI) m/z: 166.1 (M+H⁺).

### Example B13

A solution of hydrazine hydrate (459 mg, 9.16 mmol) in ethanol (5 mL) was added to a solution of ethyl 3-ethoxy-2-(trifluoroacetyl)acrylate (2.00 g, 8.33 mmol) in ethanol (15 mL) at 0° C. The reaction was allowed to warm to RT and stirred for 24 hrs. The reaction was concentrated in vacuo, dissolved in ethyl acetate (30 mL), washed with 5% citric acid (25 mL), saturated sodium bicarbonate (25 mL) and brine (25 mL), dried (Na₂SO₄) and concentrated in vacuo to afford ethyl 3-(trifluoromethyl)-1H-pyrazole-4-carboxylate (1.365 g, 79% yield). ¹H NMR (300 MHz, DMSO-d₆): δ 1.24 (t, 3H), 4.22 (q, 2H), 8.56 (s, 1H); MS (ESI) m/z: 209.0 (M+H⁺).

Isopropyl iodide (1.225 g, 7.21 mmol) was added to a solution of ethyl 3-(trifluoromethyl)-1H-pyrazole-4-carboxylate (500 mg, 2.402 mmol) and DIEA (652 mg, 5.04 mmol) in DMF (5 mL) and the reaction stirred at RT for 3 h and 60° C. for 3 h. The reaction was diluted with ethyl acetate (30 mL), washed with 5% citric acid (30 mL), saturated sodium bicarbonate (30 mL) and brine (30 mL), dried (Na₂SO₄) and concentrated in vacuo to give an oil. LC and LCMS showed starting material still present (˜40%). The oil was dissolved in DMF (4 mL), treated with DIEA (652 mg, 5.04 mmol), isopropyliodide (1.22 g, 7.21 mmol) and catalytic 4-dimethylaminopyridine (˜5 mg) and stirred at RT overnight. The reaction was diluted with ethyl acetate (30 mL), washed with 5% citric acid (30 mL), saturated sodium bicarbonate (30 mL) and brine (30 mL), dried (Na₂SO₄), concentrated in vacuo and purified by column chromatography (ethyl acetate/hexane) to afford ethyl 1-isopropyl-3-(trifluoromethyl)-1H-pyrazole-4-carboxylate (266 mg, 44% yield). ¹H NMR (300 MHz, DMSO-d₆): δ 1.26 (s, 9H), 1.43 (d, 6H), 4.23 (q, 2H), 4.64 (hp, 1H), 8.62 (s, 1H); MS (ESI) m/z: 251.0 (M+H⁺).

A solution of ethyl 1-isopropyl-3-(trifluoromethyl)-1H-pyrazole-4-carboxylate (266 mg, 1.06 mmol) and lithium hydroxide (102 mg, 4.25 mmol) in ethanol:water:dioxane (1:1:1, 6 mL) was warmed to 40° C. and stirred overnight. The mix cooled to RT, diluted with water (25 mL) and washed with ether (20 mL). The aqueous phase made acidic with 3N HCl (pH˜2) and extracted with ethyl acetate (2×15 mL). The combined ethyl acetate layers were washed with brine (20 mL), dried (Na₂SO₄) and concentrated in vacuo to give 1-isopropyl-3-(trifluoromethyl)-1H-pyrazole-4-carboxylic acid (199 mg, 84% yield) as a white solid. MS (ESI) m/z: 223.0.

### Example B14

In a procedure analogous to Example B6, isopropylhydrazine hydrochloride (896 mg, 8.10 mmol) and ethyl 2-acetyl-3-(dimethylaminomethylene)acrylate (1.50 g, 8.10 mmol) were combined and purified by chromatography (ethyl acetate/hexane) to afford ethyl 1-isopropyl-5-methyl-1H-pyrazole-4-carboxylate (faster elution, 537 mg), ¹H NMR (300 MHz, DMSO-d₆): δ 1.30 (t, 3H), 1.39 (d, 6H), 4.23 (q, 2H), 4.61 (hp, 1H), 7.82 (s, 1H); MS (ESI) m/z: 197.0 (M+H⁺) and ethyl 1-isopropyl-3-methyl-1H-pyrazole-4-carboxylate (slower elution, 91 mg), ¹H NMR (300 MHz, DMSO-d₆), δ 1.29 (t, 3H), 1.42 (d, 6H), 2.36 (s, 3H), 4.21 (q, 2H), 4.49 (hp, 1H), 8.24 (s, 1H); MS (ESI) m/z: 197.0 (M+H⁺).

In a procedure analogous to Example B6, ethyl 1-isopropyl-5-methyl-1H-pyrazole-4-carboxylate (537 mg, 2.74 mmol) and lithium hydroxide (459 mg, 10.95 mmol) were combined to give 1-isopropyl-5-methyl-1H-pyrazole-4-carboxylic acid (323 mg, 70% yield) as an off white solid. MS (ESI) m/z: 169.0 (M+H⁺).

### Example B15

In a procedure analogous to Example B6, ethyl 1-isopropyl-3-methyl-1H-pyrazole-4-carboxylate from Example B14 (91 mg, 0.464 mmol) and lithium hydroxide (78 mg, 1.855 mmol) were combined to afford 1-isopropyl-3-methyl-1H-pyrazole-4-carboxylic acid (62 mg, 79% yield). MS (ESI) m/z: 169.0 (M+H⁺).

### Example B16

3-nitro-5-(trifluoromethyl)pyridin-2-ol (6.80 g, 32.7 mmol) and quinoline (2.72 g, 21.06 mmol) were combined in a 200 mL round-bottom flask flask with an oversized magnetic stir bar. The assembly was cooled with an RT water bath. Phosphorus oxychloride (4.07 ml, 43.7 mmol) was cautiously added with vigorous stirring. After 5 min, the resulting gel would no longer stir. The apparatus was equipped with a reflux condenser and was transferred to a 120° C. oil bath. The gel quickly melted and stirring resumed with gentle refluxing. After 3 h, the mixture was cooled to RT and added portion wise to ice water with vigorous stirring. Sodium hydroxide was added to adjust the alkalinity to pH 8-9 and the mixture was extracted with EtOAc (2×100 mL) and CH₂Cl₂ (2×100 mL). The combined organics were dried (MgSO₄), concentrated in vacuo and chromatographed (EtOAc/CH₂Cl₂) provided 2-chloro-3-nitro-5-(trifluoromethyl)pyridine (6.65 g, 90% yield) as a yellow liquid. ¹H NMR (400 MHz, DMSO-d₆): δ 9.21 (m, 1H), 9.09 (m, 1H).

A Parr hydrogenation flask was charged with 10% Palladium on carbon, 50% wet (0.050 g, 0.023 mmol) and ethanol (10 mL). Triethylamine (1.0 ml, 3.09 mmol), 2-chloro-3-nitro-5-(trifluoromethyl)pyridine (0.70 g, 3.09 mmol) and an additional 10 mL of ethanol were added. The flask was purged of air, charged with 48 psi of hydrogen, and shaken for 6 h. The reaction mixture was purged of hydrogen in vacuo and filtered through Celite®, washing with EtOAc (20 mL) and EtOH (20 mL). The filtrate was concentrated in vacuo and the product npartitioned between EtOAc (40 mL) and water (20 mL). The organics were washed with sat aq NaHCO3 (20 mL) and brine (20 mL), dried (MgSO₄) and concentrated in vacuo to provide 5-(trifluoromethyl)pyridin-3-amine (498 mg, 99% yield). ¹H NMR (400 MHz, DMSO-d₆): δ 8.14 (m, 1H), 8.00 (s, 1H), 7.13 (m, 1H), 5.84 (s, 2H); MS (ESI) m/z 163.0 (M+H⁺).

### Example B17

5-Bromopyridin-3-amine (0.433 g, 2.5 mmol), 4,4,5,5-tetramethyl-2-(prop-1-en-2-yl)-1,3,2-dioxaborolane (0.630 g, 3.75 mmol), Cs₂CO₃ (3.10 g, 9.5 mmol) and Pd(PPh₃)₄ (0.289 g, 0.25 mmol) were suspended in DMF/H₂O (3:1, 20 mL). The reaction mixture was degassed with N₂ and heated at 90° C. for 16 h. Solvent was removed under reduced pressure. The residue was diluted with H₂O (20 mL) and extracted with EtOAc (3×50 mL). The combined organic layers were washed with brine (20 mL), dried, concentrated in vacuo and purified by chromatography to afford 5-(prop-1-en-2-yl)pyridin-3-amine (0.773 g, 230%) as a dark yellow oil. MS (ESI) m/z: 135.0 (M+H⁺).

To a solution of 5-(prop-1-en-2-yl)pyridin-3-amine (0.773 g, 2.48 mmol) in ethanol (8 mL) was added 10% Pd/C (0.132 g, 0.124 mmol) and the resulting suspension was stirred under a hydrogen atmosphere (1 atm) for 18 h. The reaction was filtered through Celite° and washed forward with EtOH. The filtrate was concentrated, diluted with EtOAc (30 mL) and washed with H₂O (1×15 ml) and brine (1×15 ml). The aqueous phase was back-extracted with EtOAc (1×20 ml). The combined organic layers were dried (MgSO₄) and concentrated to afford 5-isopropylpyridin-3-amine (0.453 g, 134%) as a light yellow oil. MS (ESI) m/z: 137.1 (M+H⁺).

### Example B18

A mixture of cyclopentanone (20 g, 238 mmol) and hydrazinecarboxylic acid tert-butyl ester (31.4 g, 0.238 mol) in MeOH (300 mL) was stirred at RT for 2 h. The reaction mixture was concentrated in vacuo and the resulting solid was dried under vacuum to give 1-cyclopentylidenehydrazine-2-carboxylic acid tert-butyl ester (47.1 g, 100% yield).

Sodium cyanoborohydride (6.4 g, 0.101 mol) was added portion-wise to a suspension of 1-cyclopentylidenehydrazine-2-carboxylic acid tert-butyl ester (20 g, 0.101 mol) in a mixture of acetic acid and methanol (288 mL, 1:1). The resulting solution was stirred at RT for 2 h. The reaction mixture was neutralized with 1 N aq NaOH and extracted with CH₂Cl₂. The organic layer was washed with saturated NaHCO₃, dried (Na₂SO₄) and concentrated under reduced pressure to give 1-cyclopentylhydrazine-2-carboxylic acid tert-butyl ester (18.4 g) as an oil.

To a solution of 1-cyclopentylhydrazine-2-carboxylic acid tert-butyl ester (18.4 g, 92 mmol) in a mixture of ethanol (300 mL) and conc. HCl (7.7 mL, 92 mmol) was added ethyl 2-acetyl-3-(dimethylamino)acrylate (25.5 g, 0.138 mol). The resulting mixture was refluxed for 2 h. The reaction was concentrated in vacuo, dissolved in CH₂Cl₂ (300 mL), washed with satd NaHCO₃, and brine, dried (Na₂SO₄), concentrated in vacuo and purified by chromatography on silica gel to give ethyl 1-cyclopentyl-5-methyl-1H-pyrazole-4-carboxylate (15.6 g, 76% yield). ¹H NMR (400 MHz, DMSO-d₆): δ 8.15 (s, 1H), 4.61 (m, 1H), 4.15 (q, J=8 Hz, 2H), 2.29 (s, 3H), 2.04-1.97 (m, 2H), 1.89-1.85 (m, 2H), 1.78-1.71 (m, 2H), 1.62-1.59 (m, 2H), 1.23 (t, J=8 Hz, 3H).

A solution of ethyl 1-cyclopentyl-5-methyl-1H-pyrazole-4-carboxylate (15.5 g, 70 mmol) in EtOH (200 mL) was treated with a solution of LiOH (6 g, 250 mmol) in water (100 mL) and the resultant mixture was stirred at 60° C. overnight. The reaction was concentrated in vacuo and the residue was partitioned between EtOAc and water. The aqueous layer was acidified with aq HCl (2 M) to pH 3 and was extracted with EtOAc. The extract was concentrated under reduced pressure to give 1-cyclopentyl-5-methyl-1H-pyrazole-4-carboxylic acid (8.7 g, 64% yield). ¹H NMR (300 MHz, DMSO-d₆): δ 12.05 (br s, 1H), 8.10 (s, 1H), 4.60 (m, 1H), 2.28 (s, 3H), 2.04-1.97 (m, 2H), 1.89-1.85 (m, 2H), 1.78-1.71 (m, 2H), 1.62-1.59 (m, 2H); MS (ESI) m/z: 194.99 [M+H]⁺.

### Example B19

A solution of 2,4-dinitrobenzenesulfonic acid (16.5 g, 62.0 mmol) in minimum quantity of CH₃CN was added at once to a translucent solution of iodobenzene diacetate (10 g, 31.0 mmol) in CH₃CN (100 mL). The reaction mixture was stirred for 1 hour at RT. The solution was chilled in ice and then the solution was kept in freezer. The solid was filtered and washed with Et₂O to obtain [hydroxy(2,4-dinitrobenzenesulfonyloxy)iodo]benzene (HDNIB) (13.9 g, 96% yield). ¹H NMR (400 MHz, DMSO-d₆): δ 9.91 (brs, 1H), 8.71 (d, J=2.4 Hz, 1H), 8.56 (dd, J=2.0, and 8.4 Hz, 1H), 8.38 (m, 2H), 8.24 (d, J=8.4 Hz, 1H), 7.88 (m, 1H), 7.77 (m, 2H).

A solution of ethyl pyruvate (2.0 g, 17.2 mmol) and HDNIB (9.7 g, 20.7 mmol) in trimethylacetonitrile (15 mL) was heated to reflux for 3 hours. After the reaction mixture was cooled to RT, 2,6-lutidine (0.2 mL, 1.7 mmol) was added. The reaction mixture was refluxed for an additional 8 hours. The reaction was checked by LC-MS and the solvent was removed. The residue was dissolved in CH₂Cl₂, washed with water and brine, dried (Na₂SO₄), concentrated in vacuo and purified via silica gel column chromatography (EtOAc/hexane) to obtain ethyl 2-tert-butyloxazole-5-carboxylate (1.0 g, 29% yield). ¹H NMR (400 MHz, DMSO-d₆): δ 8.89 (s, 1H), 4.42 (d, J=7.2 Hz, 2H), 1.49 (s, 9H), 1.43 (d, J=7.2 Hz, 3H); MS (ESI) m/z: 198.1 (M+H⁺).

To a stirring suspension of ethyl 2-tert-butyloxazole-5-carboxylate (1.0 g, 5.07 mmol) in 1:1:1 THF/EtOH/H₂O (15 ml) at RT was added LiOH.H₂O (486 mg) and the mixture was stirred at RT for 3 hours. The reaction mixture was checked by LC-MS and the completed reaction was concentrated to an aqueous residue, acidified (pH 3-4) with 3M HCl and extracted with EtOAc (3×). The combined organics were washed with brine (1×), dried (MgSO₄) and evaporated to afford desired product, 2-tert-butyloxazole-5-carboxylic acid (0.67 g, 78% yield). ¹H NMR (400 MHz, DMSO-d₆): δ 12.9 (brs, 1H), 8.62 (s, 1H), 1.30 (s, 9H); (ESI) m/z: 170.0 (M+H⁺).

### Example B20

To a solution of 1-tert-butyl-1H-pyrrole-3-carbaldehyde (0.339 g, 2.24 mmol) in acetone (40 mL) was added, over a 2 h period, a solution of KMnO₄ (0.708 g, 4.48 mmol) in Acetone/H₂O (1:1, 60 mL). After 3 h, the reaction was poured into a solution of 10% NaHSO₃/1N HCl (120 mL) and the solution was extracted with DCM (3×60 mL). The combined extracts were washed with H₂O (2×60 mL) and 5% NaHCO₃ (3×60 mL). The bicarbonate washes were carefully acidified to pH 3 and extracted with DCM (3×60 mL). The combined organic layers were washed with brine (1×), dried (MgSO₄) and concentrated afford 1-tert-butyl-1H-pyrrole-3-carboxylic acid (0.270 g, 72% yield) as a white solid. MS (ESI) m/z: 168.1 (M+H⁺).

### Example B21

A 60% Sodium hydride (5.16 g, 129 mmol) slurry in benzene (20 mL) was warmed to 80° C. for 15 min and then treated sequentially and dropwise (over 15 min.), first with a solution of propionitrile (7.11 g, 129 mmol) and second with a solution of methyl trimethylacetate (7.50 g, 64.6 mmol). The mixture was stirred at 80° C. overnight. The reaction was cooled to RT, quenched with i-propanol (25 mL) and water (25 mL) and diluted with ethyl acetate (50 mL). The mixture was acidified (6N HCl, pH˜=1) and the organic phase separated. The organic phase was washed with brine (25 mL), dried (Na₂SO₄) and concentrated in vacuo to to give 2-methyl pivaloylacetonitrile as an oil.

Hydroxylamine hydrochloride (5.61 g, 81 mmol) was added portionwise to a solution of sodium hydroxide (11.62 g, 291 mmol) at 0° C. in water (40 mL). The mixture was stirred until a complete salvation occurred. To this was then added crude 2-methyl pivaloylacetonitrile, the solution was warmed to 50° C. for 4 hrs, cooled to RT and allowed to stand overnight. The white solid was collected by filtration, washed with water (4×10 mL) and air dried for 1 hr to afford 3-tert-butyl-4-methylisoxazol-5-amine (4.25 g, 42% yield). ¹H NMR (400 MHz, DMSO-d₆): δ 1.19 (s, 9H), 1.79 (s, 3H), 6.09 (br. s, 2H); MS (ESI) m/z: 155.1 (M+H⁺).

### Example B22

5-Bromopyridin-3-amine (0.94 g, 5.43 mmol), PdCl₂(PPh₃)₂ (0.076 g, 0.109 mmol) and ethynyltrimethylsilane (0.64 g, 6.52 mmol) were combined in TEA (12.0 mL). After stirring for 5 min, CuI (0.010 g, 0.054 mmol) was added. The reaction mixture was flushed with N₂ and stirred at RT overnight, followed by at 55° C. overnight. The reaction was filtered and the solid was washed with EtOAc (30 mL). The combined organics were concentrated in vacuo and purified by chromatography to afford 5-(2-(trimethylsilyl)ethynyl)pyridin-3-amine (0.279 g, 27% yield) as a white solid. MS (ESI) m/z: 191.1 (M+H⁺).

To a solution of 5-(2-(trimethylsilyl)ethynyl)pyridin-3-amine (0.279 g, 1.466 mmol) in MeOH (2.0 mL) was added K₂CO₃ (0.304 g, 2.20 mmol). The reaction was stirred at RT overnight. Solvent was removed under reduced pressure and the residue was extracted with EtOAc (2×). The combined organic layers were washed with H₂O (1×) and brine (1×), dried (MgSO₄) and concentrated to afford 5-ethynylpyridin-3-amine (0.168 g, 97%) as a light yellow solid.

5-Ethynylpyridin-3-amine (0.122 g, 1.03 mmol) and 10% Pd/C (0.11 g, 0.102 mmol) were suspended in MeOH (15 mL). This was hydrogenated (42 psi) in a Parr hydrogenation apparatus overnight. The reaction was filtered through Celite® and washed forward with MeOH. The filtrate was concentrated to afford 5-ethylpyridin-3-amine (0.070 g, 56% yield) as a light yellow oil. ¹H NMR (400 MHz, DMSO-d₆): δ 7.72 (d, J=2.4 Hz, 1H), 7.58 (d, J=1.6 Hz, 1H), 6.71 (t, J=2.0 Hz, 1H), 5.16 (s, 2H), 2.43 (q, J=7.2 Hz, 2H), 1.11 (t, J=7.6 Hz, 3H).

### Example B23

In ethanol (5 mL) was placed the t-butylhydrazine hydrochloride (0.79 g, 6.3 mmol) and ethyl 2-acetyl-3-(dimethylaminomethylene)acrylate (1.0 g, 6.3 mmol). The mixture was refluxed for 8 hours. The mix was evaporated at reduced pressure to give an oil. The oil was dissolved in ether (25 mL) and washed successively with water (25 mL), saturated sodium bicarbonate (25 mL) and brine (25 mL) was dried (Na₂SO₄), concentrated in vacuo and purified by silica gel column chromatography (EtOAc/hexanes) to obtain ethyl 1-tert-butyl-5-methyl-1H-pyrazole-3-carboxylate (0.60 g, 45% yield). ¹H NMR (400 MHz, DMSO-d₆): δ 6.54 (s, 1H), 4.22 (q, J=7.2 Hz, 2H), 2.44 (s, 3H), 2.42 (s, 3H), 1.57 (s, 9H), 1.25 (t, J=7.2 Hz, 3H); MS (ESI) m/z: 211.1 (M+H⁺).

To a solution of ethyl 1-tert-butyl-5-methyl-1H-pyrazole-3-carboxylate (0.60 g, 2.85 mmol) in a mix of ethanol:water:dioxane (1:1:1, 9 mL) was added lithium hydroxide (0.48 mg, 11.4 mmol). The mixture was stirred at 40° C. for 5 hours. The solution was checked by LC-MS and diluted with water (10 mL) and the pH adjusted to ˜2 with 1N HCl. The solution was extracted with EtOAc (2×10 mL) and the combined organic phases washed with brine (20 mL), dried (Na₂SO₄), and concentrated in vacuo to obtain 1-tert-butyl-5-methyl-1H-pyrazole-3-carboxylic acid (0.50 g, 96% yield). ¹H NMR (400 MHz, DMSO-d₆): δ 12.4 (s, 1H), 5.47 (s, 1H), 2.42 (s, 3H), 1.56 (s, 9H); MS (ESI) m/z: 183.1 (M+H⁺).

### Example B24

4-nitroimidazole (0.500 g, 4.42 mmol), 2-iodopropane (0.553 ml, 5.53 mmol) and powdered K₂CO₃ (0.917 g, 6.63 mmol) were combined and stirred in DMF (25 ml) at 50° C. After 5 h, the reaction was cooled to RT. The reaction was diluted with EtOAc and filtered to remove inorganic salts, rinsing forward with EtOAc. The filtrate was evaporated to near dryness. The residue was diluted in EtOAc, washed with H₂O (2×) and brine (1×), dried (MgSO₄) and evaporated to afford 1-isopropyl-4-nitro-1H-imidazole (0.66 g 96% yield) as a pale yellow oil. ¹H NMR (400 MHz, DMSO-d₆): δ 8.51 (s, 1H), 7.98 (s, 1H), 4.52-4.49 (m, 1H), 1.44 (d, 6H); MS (ESI) m/z: 156.0 (M+H⁺), 178.0 (M+Na⁺).

1-isopropyl-4-nitro-1H-imidazole (0.66 g, 4.25 mmol) was hydrogenated (1 atm) over 10% Pd/C (50% w/w H₂O) (0.905 g, 0.425 mmol) in EtOAc (43 ml) overnight. The completed reaction was filtered through Celite®, rinsing forward with EtOAc (30-35 ml). The combined filtrates containing 1-isopropyl-1H-imidazol-4-amine were used directly in the next reaction. MS (ESI) m/z: 126.1 (M+H⁺).

To a stirring solution of 1-isopropyl-1H-imidazol-4-amine (0.532 g, 4.25 mmol) in EtOAc (70 ml) was added Troc-Cl (0.614 ml, 4.46 mmol) followed by satd. NaHCO₃ (17.23 ml, 12.75 mmol). The biphasic mixture was stirred briskly at RT. After 6 h, the layers were separated and the aqueous was extracted with EtOAc (1×). The combined organics were washed with satd. NaHCO₃ (1×) and brine (1×), dried, evaporated and triturated (EtOAc/hexanes). The solids were collected by filtration, rinsed with hexanes and dried on the filter to afford 2,2,2-trichloroethyl 1-isopropyl-1H-imidazol-4-ylcarbamate (0.392 g, 31% yield) as a pink-orange solid. ¹H NMR (400 MHz, DMSO-d₆): δ 10.2 (s, 1H), 7.49 (s, 1H), 7.02 (s, 1H), 4.80 (s, 2H), 4.3-4.25 (m, 1H), 1.35 (d, 6H); MS (ESI) m/z: 300.0 (M+FI'), 302.0 (M+2+H⁺).

### Example B25

A solution of 2-chloro-3-nitro-5-(trifluoromethyl)pyridine from Example B16 (400 mg, 1.766 mmol) in THF (5 mL) was treated sequentially with dimethyl malonate (250 μl, 2.187 mmol) and sodium hydride (60%, 85 mg, 2.119 mmol). The resultant mixture was stirred at RT overnight. The mixture was diluted with EtOAc and washed with 0.1 M aq HCl, water, and brine, dried (MgSO₄), concentrated in vacuo and purified by silica gel chromatography to provide dimethyl 2-(3-nitro-5-(trifluoromethyl)pyridin-2-yl)malonate (320 mg, 56% yield) of sufficient purity for the next step. MS (ESI) m/z: 323.0 (M+H⁺).

Dimethyl 2-(3-nitro-5-(trifluoromethyl)pyridin-2-yl)malonate (320 mg, 0.993 mmol) was combined with aq HCl (3 M, 5 mL, 15.00 mmol) and the mixture was heated to reflux overnight. The reaction mixture was cooled to RT and poured into EtOAc. Aqueous NaOH (2 M, 10 mL, 20 mmol) was added and the organic layer was separated and washed with water and brine, dried (MgSO₄) and concentrated in vacuo to provide 2-methyl-3-nitro-5-(trifluoromethyl)pyridine (53 mg, 9% yield). ¹H NMR (400 MHz, DMSO-d₆): δ 9.19 (s, 1H), 8.80 (s, 1H), 2.82 (s, 3H).

2-Methyl-3-nitro-5-(trifluoromethyl)pyridine (51 mg, 0.247 mmol) and 10% Pd/C, (50% wet, 10 mg, 4.70 μmol) in EtOH (10 mL) were combined in a Parr hydrogenation flask. The reaction mixture was purged of air under vacuum and pressurized with hydrogen (33 psi). The flask was shaken for 18 h. An additional portion of 10% Pd/C, (50% wet, 20 mg, 9.40 μmol) was added and the mixture was hydrogenated (40 psi) overnight. The reaction mixture was filtered through Celite® and the filter cake was washed with EtOH. The combined filtrate and washings were concentrated in vacuo and purified by silica gel chromatography to provide 2-methyl-5-(trifluoromethyl)pyridin-3-amine (17 mg, 39% yield). ¹H NMR (400 MHz, DMSO-d₆): δ 7.93 (s, 1H), 7.13 (s, 1H), 5.56 (s, 2H), 2.31 (s, 3H); MS (ESI) m/z: 177.0 (M+H⁺).

### Example B26

Using a procedure analogous to Example B27, 2-tert-butyl-4-chloropyrimidine-5-carboxylate from Example B27 (0.30 g, 1.24 mmo) and tert-butyl piperazine-1-carboxylate (1.15 g, 6.18 mmol) in presence of NMP (catalytic amount) were combined to afford 4-(4-(tert-butoxycarbonyl)piperazin-1-yl)-2-tert-butylpyrimidine-5-carboxylic acid (0.36 g, 80% yield). MS (ESI) m/z: 365.0 (M+H⁺).

### Example B27

In ethanol (40 mL) was placed t-butylcarbamidine hydrochloride (3.71 g, 27.2 mmol). This was treated with 21% sodium ethoxide in ethanol (8.80 g, 27.2 mmol) and stirred at RT for 15 min. To this was added the diethyl ethoxymethylenemalonate (5.87 g, 27.2 mmol) and the reaction mixture was stirred overnight at RT. The reaction mixture was refluxed for 1 hour and then cooled to RT. The solution was evaporated, the residue dissolved in water (100 mL) and the pH adjusted to 3-4 (wet litmus) with acetic acid. The mixture formed a precipitate. The solid collected by filtration, washed with water (50 mL) and dried in vacuo to obtain ethyl 2-tert-butyl-4-hydroxypyrimidine-5-carboxylate (2.18 g, 36% yield). ¹H NMR (400 MHz, DMSO-d₆): δ 12.6 (brs, 1H), 8.44 (s, 1H), 4.20 (q, J=7.2 Hz, 2H), 1.25 (s, 9H), 1.23 (t, J=7.2 Hz, 3H); MS (ESI) m/z: 225.0 (M+H⁺).

In cold (˜0° C.) POCl₃ (20 mL) was dropped triethylamine (0.55 mL) with stirring. To this was added in parts ethyl 2-tert-butyl-4-hydroxypyrimidine-5-carboxylate (2.18 g, 9.72 mmol). The mixture then warmed to 40° C. and stirred under Argon for 1 hour. The mixture was evaporated until free of POCl₃, diluted with CHCl₃ (100 mL) and poured carefully into ice (300 mL). The solution was stirred until it reached RT. The organic phase was separated, washed with sodium bicarbonate (100 mL), water (100 mL), dried (Na₂SO₄) and concentrated in vacuo to give ethyl 2-tert-butyl-4-chloropyrimidine-5-carboxylate (2.0 g, 85% yield). ¹H NMR (400 MHz, DMSO-d₆): δ 9.12 (s, 1H), 4.34 (q, J=6.8 Hz, 2H), 1.33 (s, 9H), 1.27 (t, J=6.8 Hz, 3H); MS (ESI) m/z: 243.0 (M+H⁺).

To a solution of ethyl 2-tert-butyl-4-chloropyrimidine-5-carboxylate (0.30 g, 1.24 mmol) in NMP (3 mL) was added morpholine (0.54 g, 6.16 mmol) and it was heated at 80° C. for 1.5 hour. The reaction was checked by LC-MS, water was added and the solution was extracted with ethyl acetate (3×). The organic layer was washed with brine, dried (Na₂SO₄) and solvent was removed to obtain tert-butyl 4-(5-(3-tert-butyl-5-(ethoxycarbonyl)-1H-pyrazol-1-yl)pyridin-2-yl)piperazine-1-carboxylate. MS (ESI) m/z: 294.0 (M+H⁺).

To a stirring suspension of ethyl 2-tert-butyl-4-morpholinopyrimidine-5-carboxylate (0.36 g, 1.24 mmol) in 1:1:1 THF/EtOH/H₂O (9 ml) at RT was added LiOH.H₂O (130 mg, 4.95 mmol) and the mixture was stirred overnight at RT. The reaction mixture was checked by LC-MS and the completed reaction was concentrated to an aqueous residue, acidified (pH 3-4) with 3M HCl and the solution was extracted with EtOAc (3×). The combined organics were washed with brine (1×), dried (MgSO4), filtered and concentrated in vacuo. The crude was dissolved in isopropanol and the solids (LiCl and NaCl) were filtered and washed with isopropanol. The filtrate was concentrated to obtain the desired product, 2-tert-butyl-4-morpholinopyrimidine-5-carboxylic acid (0.15 g, 46% yield). MS (ESI) m/z: 266.0 (M+H⁺).

### Example B28

3-Nitro-5-(trifluoromethyl)pyridin-2-ol (6.80 g, 32.7 mmol) and quinoline (2.72 g, 21.06 mmol) were combined in a 200 mL round-bottom flask with an oversized magnetic stir bar. The assembly was cooled with an RT water bath. Phosphorus oxychloride (4.07 ml, 43.7 mmol) was cautiously added with vigorous stirring. After 5 min, the resulting gel would no longer stir. The apparatus was equipped with a reflux condenser and was transferred to a 120° C. oil bath. The gel quickly melted and stirring resumed with gentle refluxing. After 3 h, the mixture was cooled to RT and added portion-wise to ice water with vigorous stirring. Sodium hydroxide was added to adjust the alkalinity to pH 8-9 and the mixture was extracted with EtOAc (2×100 mL) and CH₂Cl₂ (2×100 mL). The combined organics were dried (MgSO₄), concentrated in vacuo and purified via chromatography on silica gel (EtOAc—CH₂Cl₂) to provide 2-chloro-3-nitro-5-(trifluoromethyl)pyridine (6.65 g, 90% yield) as a yellow liquid. ¹H NMR (400 MHz, DMSO-d₆): δ 9.21 (m, 1H), 9.09 (m, 1H).

2-Chloro-3-nitro-5-(trifluoromethyl)pyridine (406 mg, 1.79 mmol), 1-methyl-4-(4,4,5,5-tetramethyl-1,3,2-dioxaborolan-2-yl)-1H-pyrazole (559 mg, 2.69 mmol), cesium carbonate (1752 mg, 5.38 mmol) and palladium tetrakis (207 mg, 0.179 mmol) were combined in DMF (3 mL) and water (1 mL). The headspace was evacuated and back-filled with nitrogen (4×). The mixture was heated to 90° C. overnight. The mixture was poured into EtOAc (40 mL) and washed with water (3×20 mL) and satd brine (3×20 mL). The organics were concentrated in vacuo and purifed by silica gel chromatography to provide 2-(1-methyl-1H-pyrazol-4-yl)-5-(trifluoromethyl)pyridin-3-amine (21 mg, 5% yield). ¹H NMR (400 MHz, DMSO-d₆): δ 8.29 (s, 1H), 8.13 (br s, 1 H), 7.98 (s, 1H), 7.40 (d, J=2.0 Hz, 1H), 5.55 (s, 2H), 3.91 (s, 3H); MS (ESI): m/z 473.0 (M+H⁺).

### Example 1

Using General Method A, Example B1 (0.072 g, 0.23 mmol) and Example A1 (0.062 g, 0.22 mmol) were combined and the resultant product purified via column chromatography to yield 1-(3-t-butylisoxazol-5-yl)-3-(2-fluoro-4-(2-(1-methyl-1H-pyrazol-4-yl)pyridin-4-yloxy)phenyl)urea, which was converted to corresponding mesylate salt (0.0685 g, 57% yield) by reacting with methanesulfonic acid (1.0 eq). ¹H NMR (DMSO-d₆): δ 10.4 (s, 1H), 8.89 (s, 1H), 8.59-8.57 (m, 2H), 8.24-8.20 (m, 2H), 7.65 (s, 1H), 7.45 (dd, J=11.6, 2.4 Hz, 1H), 7.17 (dd, J=8.8, 1.2 Hz, 1H), 7.12 (d, J=4.8 Hz, 1H), 6.09 (s, 1H), 3.93 (s, 3H), 2.33 (s, 3H), 1.26 (s, 9H); MS (ESI) m/z: 451.2 (M+H⁺).

### Example 2

Using general method C, Example B2 (0.0712 g, 0.30 mmol) and Example A1 (0.0853 g, 0.30 mmol) were combined and the resultant product purified via column chromatography to yield 1-(3-t-butyl-1-methyl-1H-pyrazol-5-yl)-3-(2-fluoro-4-(2-(1-methyl-1H-pyrazol-4-yl)pyridin-4-yloxy)phenyl)urea (0.139 g, 100% yield) as a white foam. ¹H NMR (DMSO-d₆): δ 8.99-8.95 (m, 2H), 8.58-8.56 (m, 2H), 8.28-8.23 (m, 2H), 7.65 (s, 1H), 7.42 (dd, J=11.6, 2.4 Hz, 1H), 7.14-7.11 (m, 2H), 3.91 (s, 3H), 3.61 (s, 3H), 2.32 (s, 3H), 1.20 (s, 9H); MS (ESI) m/z: 464.2 (M+H⁺).

### Example 3

In THF (10 mL) was placed Example A1 (87 mg, 0.31 mmol) and 3-trifluoromethylphenylisocyanate (60 mg, 0.32 mmol). The mixture was stirred overnight at RT. Hexane was added and then the solution was stirred for 1 h. The solid was filtered and dried under vacuum to obtain 1-(2-fluoro-4-(2-(1-methyl-1H-pyrazol-4-yl)pyridin-4-yloxy)phenyl)-3-(3-(trifluoromethyl)phenyl)urea (126 mg, 88% yield). ¹H NMR (400 MHz, DMSO-d₆): δ 9.39 (s, 1H), 8.68 (d, J=2.0 Hz, 1H), 8.36 (d, J=5.6 Hz, 1H), 8.25 (s, 1H), 8.15 (t, J=8.8 Hz, 1H), 8.08 (s, 1H), 7.96 (s, 1H), 7.51 (m, 2H), 7.32 (m, 1H), 7.26 (dd, J=2.8, and 12.0 Hz, 1H), 7.23 (d, J=2.4 Hz, 1H), 7.01 (dt, J=1.2, and 8.8 Hz, 1H), 6.67 (dd, J=2.4, and 5.6 Hz, 1H), 3.84 (s, 3H); LC-MS (EI) m/z: 472.0 (M+H⁺).

### Example 4

Using general method B, 5-t-butylisoxazol-3-amine (60 mg, 0.27 mmol) and Example A1 (76 mg, 0.27 mmol) were combined and the resultant product purified via column chromatography to yield 1-(5-t-butylisoxazol-3-yl)-3-(2-fluoro-4-(2-(1-methyl-1H-pyrazol-4-yl)pyridin-4-yloxy)phenyl)urea (40 mg, 38% yield). ¹H NMR (400 MHz, DMSO-d₆): δ 9.83 (s, 1H), 8.83 (br s, 1H), 8.36 (d, J=5.6 Hz, 1H), 8.25 (s, 1H), 8.15 (t, J=9.2 Hz, 1H), 7.96 (s, 1H), 7.27 (dd, J=2.8, and 11.6 Hz, 1H), 7.22 (d, J=2.4 Hz, 1H), 7.01 (m, 1H), 6.67 (dd, J=2.8, and 6.0 Hz, 1H), 6.47 (s, 1H), 3.84 (s, 3H), 1.28 (s, 9H); LC-MS (EI) m/z: 451.2 (M+H⁺).

### Example 5

Using General Method B, Example B3 (0.061 g, 0.27 mmol), and Example A1 (0.078, 0.27 mmol) were combined and the resultant product purified via column chromatography to yield 1-(1-t-butyl-1H-pyrazol-4-yl)-3-(2-fluoro-4-(2-(1-methyl-1H-pyrazol-4-yl)pyridin-4-yloxy)phenyl)urea (42 mg, 34% yield) as a white solid. ¹H NMR (400 MHz, DMSO-d₆): δ 8.71 (s, 1H), 8.62 (s, 1H), 8.54-8.52 (m, 2H), 8.26 (t, J=9.2 Hz, 1H), 8.20 (s, 1H), 7.81 (s, 1H), 7.58 (brs, 1H), 7.42 (s, 1H), 7.37-7.34 (m, 1H), 7.09-7.06 (m, 2H), 3.90 (s, 3H), 2.28 (s, 3H), 1.47 (s, 9H); MS (ESI) m/z: 450.2 (M+H⁺).

### Example 6

Using General Method A and purification via chromatography (ethyl acetate/hexane), 3-trifluoromethyl-5-aminopyridine (250 mg, 1.54 mmol) was converted to 2,2,2-trichloroethyl 5-(trifluoromethyl)pyridin-3-ylcarbamate (215 mg, 41% yield) and isolated as a thick oil. MS (ESI) m/z: 339.0 (M+H⁺).

Using General Method A, 2,2,2-trichloroethyl 5-(trifluoromethyl)pyridin-3-ylcarbamate (215 mg, 0.637 mmol) and Example A2 (170 mg, 0.637 mmol) were combined and purified by reverse phase chromatography (C18-25 column, acetonitrile/water/0.1% TFA) to give a foam. The residue was treated with 10% potassium carbonate (2 mL) and the mix extracted with ethyl acetate (2×25 mL). The combined organic phases were washed with brine, dried (Na₂SO₄) and concentrated in vacuo to afford 1-(4-(2-(1-methyl-1H-pyrazol-4-yl)pyridin-4-yloxy)phenyl)-3-(5-(trifluoromethyl)pyridin-3-yl)urea (121 mg, 41% yield). ¹H NMR (300 MHz, DMSO-d₆): δ 3.84 (s, 3H), 6.58-6.60 (m, 1H), 7.13 (d, 2H), 7.20 (s, 1H), 7.57 (d, 2H), 7.94 (s, 1H), 8.23 (s, 1H), 8.33 (d, 1H), 8.42 (s, 1H), 8.54 (s, 1H), 8.78 (s, 1H), 9.13 (s, 1H), 9.29 (s, 1H); MS (ESI) m/z: 455.3 (M+H⁺).

### Example 7

Using General Method B, the prop-1-en-2-yl carbamate of Example B4 (60 mg, 0.25 mmol) and Example A1 (72 mg, 0.25 mmol) in presence of N-methylpyrrolidine (catalytic amount) were combined and the resultant product purified via tituration with methylene chloride and filtration to afford 1-(2-fluoro-4-(2-(1-methyl-1H-pyrazol-4-yl)pyridin-4-yloxy)phenyl)-3-(3-(trifluoromethyl)isoxazol-5-yl)urea (80 mg, 68% yield). ¹H NMR (400 MHz, DMSO-d₆): δ 11.0 (s, 1H), 8.90 (brs, 1H), 8.36 (d, J=6.0 Hz, 1H), 8.24 (s, 1H), 8.04 (t, J=9.2 Hz, 1H), 7.94 (s, 1H), 7.28 (dd, J=2.8, and 11.6 Hz, 1H), 7.23 (d, J=2.4 Hz, 1H), 7.03 (m, 1H), 6.67 (dd, J=2.4, and 5.6 Hz, 1H), 6.49 (s, 1H), 3.83 (s, 3H); MS (ESI) m/z: 463.0 (M+H⁺).

### Example 8

Prop-1-en-2-yl 1-tert-butyl-1H-pyrazol-4-ylcarbamate (0.074 g, 0.331 mmol), synthesized from Example B3 using General Method E, was reacted with Example A9 (0.100 g, 0.331 mmol) in presence of N-methylpyrrolidine (0.005 g, 0.06 mmol) in dioxane (2 ml) at 80° C. for 15 hours. The completed reaction was concentrated in vacuo and purified via recrystallization (hexanes/ethyl acetate) to provide 1-(1-tert-butyl-1H-pyrazol-4-yl)-3-(2,3-difluoro-4-(2-(1-methyl-1H-pyrazol-4-yl)pyridin-4-yloxy)phenyl)urea (0.102 g, 66% yield). ¹H NMR (400 MHz, DMSO-d₆): δ 8.71 (brs, 1H), 8.69 (s, 1H), 8.34 (d, J=6 Hz, 1H), 8.24 (s, 1H), 7.97 (m, 1H), 7.95 (s, 1H), 7.79 (s, 1H), 7.40 (s, 1H), 7.23 (d, J=2.2 Hz, 1H), 7.12 (m, 1H), 6.69 (dd, J=5.5, 2.5 Hz, 1H), 3.82 (s, 3H), 1.45 (s, 9H); MS (ESI) m/z: 468.0 (MAI).

### Example 9

Using general method C, Example B5 (60 mg, 0.25 mmol) and Example A1 (72 mg, 0.25 mmol) in presence of DPPA (60 μL, 0.25 mmol) and (39 μL, 0.25 mmol) were combined and the resultant product purified via column chromatography (CH₂Cl₂/MeOH) to afford 1-(1-tert-butyl-5-(trifluoromethyl)-1H-pyrazol-4-yl)-3-(2-fluoro-4-(2-(1-methyl-1H-pyrazol-4-yl)pyridin-4-yloxy)phenyl)urea (75 mg, 57% yield). ¹H NMR (400 MHz, DMSO-d₆): δ 9.10 (brs, 1H), 8.53 (s, 1H), 8.35 (d, J=6.0 Hz, 1H), 8.24 (s, 1H), 8.18 (t, J=8.8 Hz, 1H), 7.94 (m, 2H), 7.24 (dd, J=2.4, and 11.6 Hz, 1H), 7.20 (d, J=2.4 Hz, 1H), 6.98 (m, 1H), 6.66 (dd, J=2.4, and 5.6 Hz, 1H), 3.83 (s, 3H), 1.57 (s, 9H); MS (ESI) m/z: 518.0 (M+H⁺).

### Example 10

Using General Method C, Example B6 (50 mg, 0.27 mmol) and Example A1 (78 mg, 0.27 mmol) in presence of DPPA (65 μL, 0.27 mmol) and (42 μL, 0.27 mmol) were combined and the resultant product purified via column chromatography (CH₂Cl₂/MeOH) to afford 1-(1-tert-butyl-5-methyl-1H-pyrazol-4-yl)-3-(2-fluoro-4-(2-(1-methyl-1H-pyrazol-4-yl)pyridin-4-yloxy)phenyl)urea (55 mg, 43% yield). ¹H NMR (400 MHz, DMSO-d₆): δ 8.57 (brs, 1H), 8.35 (d, J=5.6 Hz, 1H), 8.25 (s, 1H), 8.20 (t, J=9.2 Hz, 1H), 8.15 (s, 1H), 7.96 (s, 1H), 7.44 (s, 1H), 7.22 (m, 2H), 6.97 (m, 1H), 6.66 (dd, J=2.4, and 5.6 Hz, 1H), 3.84 (s, 3H), 2.31 (s, 3H), 1.54 (s, 9H); MS (ESI) m/z: 464.2 (M+H⁺).

### Example 11

Using general method D, 2-amino-5-t-butyl-1,3,4-thiadiazole (0.5000 g, 3.2 mmol) was converted to prop-1-en-2-yl 5-tert-butyl-1,3,4-thiadiazol-2-ylcarbamate (0.73 g, 95% yield) as a beige solid which was used as is in the next reaction. ¹H NMR (400 MHz, acetone-d₆): δ 4.77-4.66 (m, 2H), 1.95 (s, 3H), 1.38 (s, 9H); MS (ESI) m/z: 242.3 (M+H⁺).

Prop-1-en-2-yl 5-tert-butyl-1,3,4-thiadiazol-2-ylcarbamate (60 mg, 0.249 mmol), Example A1 (70.7 mg, 0.249 mmol), and 1-methylpyrrolidine (1.293 μl, 0.012 mmol) were combined in THF (2.5 ml) and stirred with heating at 70° C. overnight in a sealed screw-cap vial. The completed reaction was cooled to RT and purified directly by reverse phase chromatography to afford 1-(5-tert-butyl-1,3,4-thiadiazol-2-yl)-3-(2-fluoro-4-(2-(1-methyl-1H-pyrazol-4-yl)pyridin-4-yloxy)phenyl)urea (84 mg, 72% yield) as an off-white solid following lyophilization. ¹H NMR (400 MHz, DMSO-d₆): δ 9.04 (brs, 1H), 8.54-8.52 (m, 1H), 8.48 (brs, 1H), 8.2-8.16 (m, 2H), 7.54 (brs, 1H), 7.44-7.40 (m, 1H), 7.15-7.13 (m, 1H), 7.01-7.00 (m, 1H), 3.91 (s, 3H), 1.39 (s, 9H); MS (ESI) m/z: 438.0 (M+H⁺).

### Example 12

Using General Method C, Example B8 (0.15 g, 0.63 mmol), Example A1 (0.15 g, 0.53 mmol) in presence of triethylamine (0.16 g, 1.58 mmol) and DPPA (0.29 g, 1.05 mmol) were combined to afford 1-(3-tert-butyl-1-(2-(dimethylamino)ethyl)-1H-pyrazol-5-yl)-3-(2-fluoro-4-(2-(1-methyl-1H-pyrazol-4-yl)pyridin-4-yloxy)phenyl)urea (0.085 g, 31% yield) as a white solid. ¹H NMR (400 MHz, DMSO-d₆): δ 9.23 (s, 1H), 9.07 (s, 1H), 8.41 (d, J=5.6 Hz, 1H), 8.29 (s, 1H), 8.15 (t, J=9.2 Hz, 1H), 8.00 (s, 1H), 7.31-7.27 (m, 2H), 7.04 (dt, J=9.2 Hz, 1.2 Hz, 1H), 6.71 (dd, J=5.6 Hz, 2.0 Hz, 1H), 6.11 (s, 1H), 4.03 (t, J=6.8 Hz, 2H), 3.89 (s, 3H), 2.61 (t, J=6.8 Hz, 2H), 2.60 (s, 6H), 1.24 (s, 9H); MS (ESI) m/z: 521.3 (M+H⁺).

### Example 13

Using General Method B, the prop-1-en-2-yl carbamate of Example B7 (60 mg, 0.24 mmol) and Example A1 (68 mg, 0.24 mmol) in presence of N-methylpyrrolidine (catalytic amount) were combined and the resultant product purified via tituration with CH₂Cl₂ and filtration to afford 1-(3-cyclopentylisoxazol-5-yl)-3-(2-fluoro-4-(2-(1-methyl-1H-pyrazol-4-yl)pyridin-4-yloxy)phenyl)urea (71 mg, 62% yield). ¹H NMR (400 MHz, DMSO-d₆): δ 10.3 (s, 1H), 8.77 (brs, 1H), 8.37 (d, J=6.0 Hz, 1H), 8.26 (s, 1H), 8.11 (t, J=8.8 Hz, 1H), 7.96 (s, 1H), 7.28 (dd, J=2.4, and 11.6 Hz, 1H), 7.24 (d, J=2.4 Hz, 1H), 7.03 (m, 1H), 6.68 (dd, J=2.4, and 5.6 Hz, 1H), 6.02 (s, 1H), 3.85 (s, 3H), 1.95 (m, 2H), 1.62 (m, 6H), 1.26 (s, 3H); MS (ESI) m/z: 477.0 (M+H⁺).

### Example 14

Using general method B, the prop-1-en-2-yl carbamate of Example B10 (60 mg, 0.25 mmol) and Example A1 (72 mg, 0.25 mmol) in presence of N-methylpyrrolidine (catalytic amount) were combined and the resultant product purified via tituration with CH₂Cl₂ and filtration to afford 1-(2-fluoro-4-(2-(1-methyl-1H-pyrazol-4-yl)pyridin-4-yloxy)phenyl)-3-(3-(1-methylcyclopentyl)isoxazol-5-yl)urea (68 mg, 58% yield). ¹H NMR (400 MHz, DMSO-d₆): δ 10.3 (s, 1H), 8.78 (brs, 1H), 8.37 (d, J=5.6 Hz, 1H), 8.26 (s, 1H), 8.11 (t, J=9.2 Hz, 1H), 7.96 (s, 1H), 7.28 (dd, J=2.8, and 12.0 Hz, 1H), 7.24 (d, J=2.4 Hz, 1H), 7.03 (m, 1H), 6.68 (dd, J=2.8, and 6.0 Hz, 1H), 5.98 (s, 1H), 3.85 (s, 3H), 3.02 (m, 1H), 1.95 (m, 2H), 1.62 (m, 6H); MS (ESI) m/z: 463.0 (M+H⁺).

### Example 15

Using General Method C, Example B11 (60 mg, 0.33 mmol) and Example A1 (95 mg, 0.33 mmol) in presence of DPPA (79 μL, 0.33 mmol) and (51 μL, 0.33 mmol) were combined and the resultant product purified via column chromatography (CH₂Cl₂/MeOH) to afford 1-(1-cyclopentyl-1H-pyrazol-4-yl)-3-(2-fluoro-4-(2-(1-methyl-1H-pyrazol-4-yl)pyridin-4-yloxy)phenyl)urea (53 mg, 34% yield). ¹H NMR (400 MHz, DMSO-d₆): δ 8.70 (s, 1H), 8.51 (d, J=2.0 Hz, 1H), 8.37 (d, J=5.6 Hz, 1H), 8.26 (s, 1H), 8.18 (t, J=8.8 Hz, 1H), 7.96 (s, 1H), 7.78 (s, 1H), 7.22 (m, 2H), 6.99 (m, 1H), 6.67 (dd, J=2.4, and 5.6 Hz, 1H), 4.62 (m, 1H), 3.86 (s, 3H), 2.03 (m, 2H), 1.87 (m, 2H), 1.76 (m, 2H), 1.61 (m, 2H); MS (ESI) m/z: 462.3 (M+H⁺).

### Example 16

Using General Method D, Example B12 (0.20 g, 1.2 mmol) and isopropenyl chloroformate (0.15 mL) in presence of LiHMDS (1.0M, 2.5 mL) were combined to afford prop-1-en-2-yl 1-methyl-3-(trifluoromethyl)-1H-pyrazol-5-ylcarbamate (0.2 g, 67% yield). MS (ESI) m/z: 250.0 (M+H⁺).

Using General Method D, prop-1-en-2-yl 1-methyl-3-(trifluoromethyl)-1H-pyrazol-5-ylcarbamate (60 mg, 0.24 mmol) and Example A1 (68 mg, 0.24 mmol) in presence of N-methylpyrrolidine (catalytic amount) were combined and the resultant product purified via tituration with CH₂Cl₂ and filtration to afford 1-(2-fluoro-4-(2-(1-methyl-1H-pyrazol-4-yl)pyridin-4-yloxy)phenyl)-3-(1-methyl-3-(trifluoromethyl)-1H-pyrazol-5-yl)urea (51 mg, 45% yield). ¹H NMR (400 MHz, DMSO-d₆): δ 9.30 (s, 1H), 8.99 (d, J=2.4 Hz, 1H), 8.38 (d, J=5.6 Hz, 1H), 8.27 (s, 1H), 8.16 (t, J=9.2 Hz, 1H), 7.97 (s, 1H), 7.29 (dd, J=2.4, and 11.6 Hz, 1H), 7.24 (d, J=2.4 Hz, 1H), 7.04 (m, 1H), 6.69 (dd, J=2.4, and 5.6 Hz, 1H), 6.63 (s, 1H), 3.86 (s, 3H), 3.79 (s, 3H); MS (ESI) m/z: 476.0 (M+H⁺).

### Example 17

The prop-1-en-2-yl carbamate of Example B3 (0.075 g, 0.335 mmol), prepared using General Method E, was reacted with Example A4 (0.1 g, 0.335 mmol) in presence of N-methylpyrrolidine (0.006 g, 0.06 mmol) in dioxane (2 ml) at 80° C. for 15 hours. The completed reaction was concentrated in vacuo and the residue purified by flash chromatography (hexane/ethyl acetate) to provide 1-(1-tert-butyl-1H-pyrazol-4-yl)-3-(2-fluoro-3-methyl-4-(2-(1-methyl-1H-pyrazol-4-yl)pyridin-4-yloxy)phenyl)urea (0.115 g, 74% yield). ¹H NMR (400 MHz, DMSO-d₆): δ 8.74 (s, 1H), 8.52 (brs, 1H), 8.39 (d, J=6 Hz, 1H), 8.29 (s, 1H), 8.07 (t, J=9 Hz, 1H), 7.98 (s, 1H), 7.84 (s, 1H), 7.45 (s, 1H), 7.20 (d, J=2.3 Hz, 1H), 6.96 (m, 1H), 6.58 (dd, J=5.5, 2.5 Hz, 1H), 3.88 (s, 3H), 2.08 (brs, 3H), 1.52 (s, 9H); MS (ESI) m/z: 464.2 (M+H⁺).

### Example 18

Using General Method C, Example B13 (100 mg, 0.450 mmol), triethylamine (52 mg, 0.518 mmol), Example A1 (128 mg, 0.450 mmol) and DPPA (142 mg, 0.518 mmol) were combined, purified by reverse phase chromatography (C18-25 column, acetonitrile/water), treated with saturated sodium bicarbonate (10 mL) and extracted with ethyl acetate (2×20 mL). The combined organic phases washed with brine (20 mL), dried (Na₂SO₄), concentrated in vacuo, dissolved in acetonitrile/water and lyophilized to give 1-(2-fluoro-4-(2-(1-methyl-1H-pyrazol-4-yl)pyridin-4-yloxy)phenyl)-3-(1-isopropyl-3-(trifluoromethyl)-1H-pyrazol-4-yl)urea (112 mg, 49% yield). ¹H NMR (300 MHz, DMSO-d₆): δ 1.48 (d, 6H), 3.92 (s, 3H), 4.63 (hp, 1H), 6.73-6.75 (m, 1H), 7.06-7.08 (m, 1H), 7.29 (s, 1H), 7.29-7.34 (m, 1H), 8.03 (s, 1H), 8.27-8.32 (m, 3H), 8.40-8.44 (m, 1H), 8.73 (s, 1H), 9.15 (s, 1H); MS (ESI) m/z: 504.0 (MAI).

### Example 19

Using General Method C, Example B14 (150 mg, 0.892 mmol), triethylamine (104 mg, 1.026 mmol), Example A1 (254 mg, 0.892 mmol) and DPPA (282 mg, 1.026 mmol) were combined and purified by chromatography (methanol/dichloromethane) to afford 1-(2-fluoro-4-(2-(1-methyl-1H-pyrazol-4-yl)pyridin-4-yloxy)phenyl)-3-(1-isopropyl-5-methyl-1H-pyrazol-4-yl)urea (98 mg, 24% yield) as a foam. ¹H NMR (300 MHz, DMSO-d₆): δ 1.44 (d, 6H), 2.29 (s, 3H), 4.00 (s, 3H), 4.56 (hp, 1H), 7.10 (br s, 1H), 7.15-7.18 (m, 1H), 7.43-7.46 (m, 1H), 7.62 (s, 2 H), 8.30 (br s, 1H), 8.38 (t, 1H), 8.44 (s, 1H), 8.58-8.62 (m, 2H), 8.78 (br s, 1H); MS (ESI) m/z: 450.2 (M+H⁺).

### Example 20

Using General Method C, Example B15 (62 mg, 0.369 mmol), triethylamine (43 mg, 0.424 mmol), Example A1 (105 mg, 0.369 mmol) and DPPA (117 mg, 0.424 mmol) were combined and purified by column chromatography (methanol/dichloromethane) to afford 1-(2-fluoro-4-(2-(1-methyl-1H-pyrazol-4-yl)pyridin-4-yloxy)phenyl)-3-(1-isopropyl-3-methyl-1H-pyrazol-4-yl)urea (88 mg, 53% yield) as a foam. ¹H NMR (300 MHz, DMSO-d₆): δ 1.46 (d, 6H), 2.22 (s, 3H), 3.98 (s, 3H), 4.45 (hp, 1H), 6.89 (br s, 1H), 7.11-7.14 (m, 1H), 7.37-7.41 (m, 1H), 7.44 (br s, 1 H), 7.88 (s, 1H), 8.15 (br s, 1H), 8.37 (t, 1H), 8.44-8.53 (m, 3H), 8.77 (s, 1H); MS (ESI) m/z: 450.2 (M+H⁺).

### Example 21

A mixture of Example A1 (2.0 g, 7.04 mmol) and saturated aq NaHCO₃ (100 mL) in EtOAc (100 mL) was cooled in an ice bath and treated with isopropenyl chloroformate (1.6 mL, 14.64 mmol). The reaction mixture was allowed to slowly warm to RT overnight. The organic layer was separated and washed with sat aq NaHCO₃ (25 mL) and brine (25 mL), dried (MgSO₄), concentrated in vacuo and was re-crystallized (diethylether) to provide prop-1-en-2-yl 2-fluoro-4-(2-(1-methyl-1H-pyrazol-4-yl)pyridin-4-yloxy)phenylcarbamate (2.32 g, 90% yield). ¹H NMR (400 MHz, DMSO-d₆): δ 9.69 (br s, 1H), 8.38 (d, J=5.6 Hz, 1H), 8.26 (s, 1H), 7.96 (d, J=0.8 Hz, 1H), 7.67 (br t, J=8.4 Hz, 1H), 7.27 (d, J=2.4 Hz, 1H), 7.22 (dd, J=11.2, 2.4 Hz, 1 H), 7.00 (m, 1H), 6.69 (dd, J=5.6, 2.4 Hz, 1H), 4.74 (m, 1H), 4.72 (s, 1H), 3.84 (s, 3 H), 1.92 (s, 3H); MS (ESI) m/z: 369.1 (M+H⁺).

### Example B16

(81 mg, 0.500 mmol), prop-1-en-2-yl2-fluoro-4-(2-(1-methyl-1H-pyrazol-4-yl)pyridin-4-yloxy)phenylcarbamate (180 mg, 0.489 mmol) and N-methylpyrrolidine (4.25 mg, 0.050 mmol) were combined in THF (1 mL) and heated to 55° C. for 48 h. The reaction mixture was concentrated in vacuo and purified by silica gel chromatography to provide 1-(2-fluoro-4-(2-(1-methyl-1H-pyrazol-4-yl)pyridin-4-yloxy)phenyl)-3-(5-(trifluoromethyl)pyridin-3-yl)urea (168 mg, 72% yield). ¹H NMR (400 MHz, DMSO-d₆) δ 9.60 (s, 1H), 8.89 (d, J=1.7 Hz, 1H), 8.77 (d, J=2.4 Hz, 1H), 8.59 (d, J=1.0 Hz, 1H), 8.46 (t, J=2.0 Hz, 1H), 8.39 (d, J=5.8 Hz, 1H), 8.27 (s, 1H), 8.13 (t, J=9.0 Hz, 1H), 7.98 (s, 1H), 7.29 (dd, J=11.8, 2.6 Hz, 1H), 7.26 (d, J=2.5 Hz, 1H), 7.05 (m, 1H), 6.70 (dd, J=5.6, 2.2 Hz, 1H), 3.86 (s, 3H); MS (ESI): m/z 473.0 (M+H⁺).

### Example 22

Using General Method F, Example B17 (0.453 g, 2.48 mmol) was converted to prop-1-en-2-yl5-isopropylpyridin-3-ylcarbamate (0.185 g, 34%) as a white solid. ¹H NMR (400 MHz, DMSO-d₆): δ 10.10 (s, 1H), 8.44 (d, J=2.4 Hz, 1H), 8.16 (d, J=2.0 Hz, 1H), 7.84 (s, 1H), 4.77 (t, J=1.2 Hz, 1H), 4.74 (s, 1H), 2.91 (m, 1H), 1.94 (d, J=0.8 Hz, 3H), 1.21 (d, J=6.8 Hz, 6H); MS (ESI) m/z: 221.1 (M+H⁺).

Prop-1-en-2-yl 5-isopropylpyridin-3-ylcarbamate (0.053 g, 0.24 mmol), Example A1 (0.068 g, 0.238 mmol) and N-methylpyrrolidine (0.0020 g, 0.024 mmol) were combined in THF (1.0 mL). The mixture was heated at 55° C. for 12 h. Solvent was removed and the residue was purified by chromatography to afford 1-(2-fluoro-4-(2-(1-methyl-1H-pyrazol-4-yl)pyridin-4-yloxy)phenyl)-3-(5-isopropylpyridin-3-yl)urea (0.0648 g, 61% yield) as a white solid. ¹H NMR (400 MHz, DMSO-d₆): δ 9.23 (s, 1H), 8.75 (d, J=2.0 Hz, 1H), 8.45 (d, J=2.0 Hz, 1H), 8.42 (d, J=4.8 Hz, 1H), 8.31 (s, 1H), 8.22 (t, J=8.8 Hz, 1H), 8.18 (d, J=1.6 Hz, 1H), 8.02 (s, 1H), 7.90 (t, J=1.8 Hz, 1H), 7.32 (dd, J=12.0, 2.8 Hz, 1H), 7.29 (d, J=2.0 Hz, 1H), 7.06 (m, 1H), 6.73 (dd, J=5.6, 2.4 Hz, 1H), 3.90 (s, 3H), 2.97 (m, 1H), 1.27 (d, J=6.8 Hz, 6H); MS (ESI) m/z: 447.3 (M+H⁺).

### Example 23

Using General Method C, Example B18 0.133 g, 0.686 mmol), triethylamine (0.139 g, 1.372 mmol), DPPA (0.189 g, 0.686 mmol) and Example A1 (0.130 g, 0.457 mmol) were combined and the residue purified via recrystallization (acetonitrile) to afford 1-(1-cyclopentyl-5-methyl-1H-pyrazol-4-yl)-3-(2-fluoro-4-(2-(1-methyl-1H-pyrazol-4-yl)pyridin-4-yloxy)phenyl)urea (0.11 g, 50.6% yield) as a white solid. ¹H NMR (400 MHz, DMSO-d₆): δ 8.72 (s, 1H), 8.45 (m, 2H), 8.33 (m, 2H), 8.05 (s, 1H), 7.86 (s, 1H), 7.32 (m, 2H), 7.07 (m, 1H), 6.75 (dd, J=6, 2.5 Hz, 1H), 4.56 (m, 1H), 3.94 (s, 3H), 2.19 (s, 3H), 2.09-1.59 (m, 8H); MS (ESI) m/z: 476.2 (M+H⁺).

### Example 24

Using General Method A, benzo[d]isoxazol-3-amine (500 mg, 3.37 mmol) and Troc-Cl (1.185 g, 5.59 mmol) were combined, purified by column chromatography (ethyl acetate/hexanes), triturated with hexanes (30 mL), filtered and dried to afford 2,2,2-trichloroethyl benzo[d]isoxazol-3-ylcarbamate. ¹H NMR (300 MHz, DMSO-d₆): δ 5.15 (s, 2H), 7.50 (t, 1H), 7.77-7.83 (m, 2H), 8.16 (d, 1H), 11.51 (s, 1 H); MS (ESI) m/z: 310.9 (M+H⁺).

Using General Method A, 2,2,2-trichloroethyl benzo[d]isoxazol-3-ylcarbamate (109 mg, 0.352 mmol) and Example A1 (100 mg, 0.352 mmol) were combined and purified by normal phase chromatography (methanol/dichloromethane) and reverse phase chromatography (acetonitrile/water) to give a white solid. The solid was slurried in saturated sodium bicarbonate (4 mL)/ethyl acetate (15 mL), filtered, washed with water (5 mL) and ethyl acetate (5 mL) and dried to afford 1-(benzo[d]isoxazol-3-yl)-3-(2-fluoro-4-(2-(1-methyl-1H-pyrazol-4-yl)pyridin-4-yloxy)phenyl)urea (17 mg, 10% yield). ¹H NMR (300 MHz, DMSO-d₆): δ 3.96 (s, 3H), 6.85 (br s, 1H), 7.21-7.25 (m, 1H), 7.37-7.54 (m, 3H), 7.80 (br s, 2H), 8.11 (br s, 1H), 8.29-8.41 (m, 3H), 8.52 (br s, 1H), 9.56 (br s, 1H), 10.64 (br s, 1H); MS (ESI) m/z: 445.1 (M+H⁺).

### Example 25

2,2,2-trichloroethyl 3-tert-butylisoxazol-5-ylcarbamate (0.125 g, 0.397 mmol), synthesized according to General Method A from Example Bl, was reacted with Example A3 (0.100 g, 0.331 mmol) in dioxane (2 ml) in presence of N-methylpyrrolidine (0.028 g, 0.331 mmol) at 80° C. for 13 hours. The reaction mixture was concentrated in vacuo and the residue purified via recrystallization (methanol) to provide 1-(3-tert-butylisoxazol-5-yl)-3-(2,3-difluoro-4-(2-(1-methyl-1H-pyrazol-4-yl)pyridin-4-yloxy)phenyl)urea (0.043 g, 28% yield) as a white solid. ¹H NMR (400 MHz, DMSO-d₆): δ 10.54 (s, 1H), 9.10 (s, 1H), 8.52 (d, J=6 Hz, 1H), 8.42 (s, 1H), 8.12 (s, 1H), 8.06 (m, 1H), 7.41 (brs, 1H), 7.35 (m, 1H), 6.87 (dd, J=6, 2.5 Hz, 1H), 6.20 (s, 1H), 3.98 (s, 3H), 1.38 (s, 9H); MS (ESI) m/z: 469.1 (M+H⁺).

### Example 26

Using General Method C, Example B19 (50 mg, 0.30 mmol) and Example A1 (84 mg, 0.30 mmol) in presence of DPPA (70 μL, 0.30 mmol) and (45 μL, 0.30 mmol) were combined and the resultant product purified via column chromatography (CH₂Cl₂/MeOH) to afford 1-(2-tert-butyloxazol-5-yl)-3-(2-fluoro-4-(2-(1-methyl-1H-pyrazol-4-yl)pyridin-4-yloxy)phenyl)urea (22 mg, 17% yield). ¹H NMR (400 MHz, DMSO-d₆): δ 9.33 (s, 1H), 8.65 (brs, 1H), 8.36 (brd, J=5.6 Hz, 1H), 8.25 (s, 1H), 8.18 (brt, J=9.2 Hz, 1H), 7.95 (s, 1H), 7.75 (s, 2H), 7.24 (m, 1H), 7.21 (s, 1H), 6.99 (m, 1H), 6.67 (m, 1H), 3.84 (s, 3H), 1.30 (s, 9H); MS (ESI) m/z: 451.2 (M+H⁺).

### Example 27

3-Amino-5-(trifluoromethyl)pyridin-2(1H)-one (44 mg, 0.247 mmol), prop-1-en-2-yl2-fluoro-4-(2-(1-methyl-1H-pyrazol-4-yl)pyridin-4-yloxy)phenylcarbamate from Example 21 (85 mg, 0.231 mmol) and N-methylpyrrolidine (7.5 mg, 0.088 mmol) were combined in 1,4-dioxane (0.8 mL). The resultant mixture was heated to 80° C. After 13 h, the mixture was cooled to RT and diluted with ethyl acetate (3 mL). The resultant precipitate was collected by filtration, washed with ethyl acetate and dried in vacuo to provide 1-(2-fluoro-4-(2-(1-methyl-1H-pyrazol-4-yl)pyridin-4-yloxy)phenyl)-3-(2-oxo-5-(trifluoromethyl)-1,2-dihydropyridin-3-yl)urea as an off-white solid (65 mg, 58% yield). ¹H NMR (400 MHz, DMSO-d₆): δ 12.47 (s, 1 H), 9.56 (s, 1H), 9.35 (s, 1H), 8.36 (d, J=5.3 Hz, 1H), 8.25 (br s, 2H), 8.17 (t, J=9.4 Hz, 1H), 7.96 (s, 1H), 7.59 (s, 1H), 7.25-7.22 (m, 2H), 7.00 (d, J=8.5 Hz, 1H), 6.68 (m, 1H), 3.84 (s, 3H); MS (ESI) m/z: 489.1 (M+H⁺).

### Example 28

To a solution of 5-tert-butyl-2-methylfuran-3-carbonyl chloride (0.341 g, 1.699 mmol) in THF (2 ml) added lithium hydroxide (0.107 g, 2.55 mmol) in water (1 mL) and the mixture was stirred for 2 h at RT. Solvent was removed in vacuo and the residue was acidified with 2N HCl to afford solid which was filtered and air dried to afford 5-tert-butyl-2-methylfuran-3-carboxylic acid (0.29 g, 94% yield) as a white solid. MS (ESI) m/z: 183.1 (M+H⁺).

Using General Method C 5-tert-butyl-2-methylfuran-3-carboxylic acid (0.07 g, 0.37 mmol), Example A1 (0.07 g, 0.25 mmol), triethylamine (0.07 g, 0.75 mmol) and DPPA (0.13 g, 0.5 mmol) were combined to afford 1-(5-tert-butyl-2-methylfuran-3-yl)-3-(2-fluoro-4-(2-(1-methyl-1H-pyrazol-4-yl)pyridin-4-yloxy)phenyl)urea (0.065 g, 56% yield) as a white solid. ¹H NMR (400 MHz, DMSO-d₆): δ 8.60 (s, 1H), 8.36-8.34 (m, 2H), 8.24 (s, 1H), 8.17 (t, J=9.2 Hz, 1H), 7.95 (s, 1H), 7.23-7.20 (m, 2H), 6.96 (dd, J=8.8 Hz, 2.4 Hz, 1H), 6.65 (dd, J=5.6 Hz, 2.4 Hz, 1H), 6.26 (s, 1H), 3.84 (s, 3H), 2.16 (s, 3H), 1.19 (s, 9H); MS (ESI) m/z: 464.2 (M+H⁺).

### Example 29

Using General Method B, 6-fluorobenzo[d]thiazol-2-amine (2.00 g, 11.89 mmol) was converted to prop-1-en-2-yl 6-fluorobenzo[d]thiazol-2-ylcarbamate (2.00 g, 67% yield) as a white solid. ¹H NMR (400 MHz, DMSO-d₆): δ 12.33 (s, 1H), 7.86 (dd, J=9, 3 Hz, 1H), 7.69 (dd, J=9, 5 Hz, 1H), 7.24 (dt, J=9, 2.5 Hz, 1H), 4.84 (s, 1H), 4.80 (s, 3H), 1.94 (s, 3H); MS (ESI) m/z: 253.1 (M+H⁺).

Prop-1-en-2-yl 6-fluorobenzo[d]thiazol-2-ylcarbamate (0.060 g, 0.238 mmol) was reacted with Example A1 (0.068 g, 0.238 mmol) in the presence of a catalytic amount of N-methylpyrrolidine in dioxane (5 ml) at 70° C. for 3 hours. The reaction mixture was cooled and the product filtered, washed and dried to provide 1-(2-fluoro-4-(2-(1-methyl-1H-pyrazol-4-yl)pyridin-4-yloxy)phenyl)-3-(6-fluorobenzo[d]thiazol-2-yl)urea (0.08 g, 70% yield) as a white solid. ¹H NMR (400 MHz, DMSO-d₆): δ 11.03 (s, 1H), 9.15 (s, 1H), 8.38 (d, J=6 Hz, 1H), 8.26 (s, 1H), 8.15 (t, J=9 Hz, 1H), 7.96 (s, 1H), 7.85 (dd, J=9, 2.5 Hz, 1H), 7.68 (m, 1H), 7.31 (dd, J=12, 2.5 Hz, 1H), 7.24 (m, 2H), 7.04 (m, 1H), 6.69 (dd, J=6, 2.5 Hz, 1H), 3.84 (s, 3H); MS (ESI) m/z: 479.1 (M+H⁺).

### Example 30

Using General Method C, Example B20 (0.070 g, 0.419 mmol), TEA (0.088 mL, 0.628 mmol), DPPA (0.135 mL, 0.628 mmol) and Example A1 (0.119 g, 0.419 mmol) were combined to afford 1-(1-tert-butyl-1H-pyrrol-3-yl)-3-(2-fluoro-4-(2-(1-methyl-1H-pyrazol-4-yl)pyridin-4-yloxy)phenyl)urea (0.011 g, 6% yield) as a white solid. ¹H NMR (400 MHz, DMSO-d₆): δ 8.51 (s, 1H), 8.36-8.34 (m, 2H), 8.25-8.19 (m, 2H), 7.95 (s, 1H), 7.22-7.18 (m, 2H), 6.99 (t, J=2.0 Hz, 1H), 6.95 (m, 1H), 6.72 (t, J=2.8 Hz, 1H), 6.65 (dd, J=5.6, 2.4 Hz, 1H), 5.86 (t, J=2.0 Hz, 1H), 3.84 (s, 3H), 1.43 (s, 9H); MS (ESI) m/z: 449.2 (M+H⁺).

### Example 31

Using General Method A, 2,2,2-trichloroethyl 3-tert-butyl-4-methylisoxazol-5-ylcarbamate (100 mg, 0.30 mmol), prepared via General Method A from Example B21 and Example A1 (86 mg, 0.30 mmol) in presence of DIEA (0.12 mL) were combined and the resultant product purified via column chromatography (EtOAc/hexanes) to afford 1-(3-tert-butyl-4-methylisoxazol-5-yl)-3-(2-fluoro-4-(2-(1-methyl-1H-pyrazol-4-yl)pyridin-4-yloxy)phenyl)urea (65 mg, 46% yield). ¹H NMR (400 MHz, DMSO-d₆): δ 9.15 (s, 1H), 8.83 (brs, 1H), 8.36 (d, J=5.6 Hz, 1H), 8.25 (s, 1H), 8.05 (t, J=9.2 Hz, 1H), 7.96 (s, 1H), 7.26 (dd, J=2.8, and 12.0 Hz, 1H), 7.23 (d, J=2.0 Hz, 1H), 7.00 (m, 1H), 6.67 (dd, J=2.4, and 5.6 Hz, 1H), 3.84 (s, 3H), 1.96 (s, 3H), 1.29 (s, 9H); MS (ESI) m/z: 465.2 (M+H⁺).

### Example 32

A mixture of prop-1-en-2-yl 2-fluoro-4-(2-(1-methyl-1H-pyrazol-4-yl)pyridin-4-yloxy)phenylcarbamate from Example 21 (0.096 g, 0.262 mmol), Example B22 (0.032 g, 0.262 mmol) and N-methylpyrrolidine (2.23 mg, 0.026 mmol) in dioxane (1.0 mL) was heat at 70° C. overnight. Solvent was removed under reduced pressure. The residue was purified by chromatography to afford 1-(5-ethylpyridin-3-yl)-3-(2-fluoro-4-(2-(1-methyl-1H-pyrazol-4-yl)pyridin-4-yloxy)phenyl)urea (0.054 g, 47% yield) as a white solid. ¹H NMR (400 MHz, DMSO-d₆): δ 9.39 (s, 1H), 8.82 (d, J=2.0 Hz, 1H), 8.50 (d, J=2.4 Hz, 1H), 8.41 (d, J=5.6 Hz, 1H), 8.31 (s, 1H), 8.20-8.14 (m, 2H), 8.01 (s, 1H), 7.88 (d, J=2.0 Hz, 1H), 7.31-7.27 (m, 2H), 7.04 (d, J=9.2 Hz, 1H), 6.74 (dd, J=5.6, 2.6 Hz, 1H), 3.87 (s, 3H), 2.64 (q, J=7.6 Hz, 2H), 1.21 (t, J=7.6 Hz, 3H); MS (ESI) m/z: 433.1 (M+H⁺).

### Example 33

To a solution of 3-cyclopropyl-1-methyl-1H-pyrazol-5-amine (60 mg, 0.434 mmol) in dioxane (1 mL) was added prop-1-en-2-yl 2-fluoro-4-(2-(1-methyl-1H-pyrazol-4-yl)pyridin-4-yloxy)phenylcarbamate from Example 21 (0.16 g, 0.434 mmol), and DBU (6.61 mg, 0.043 mmol) and the mixture was stirred overnight at 70° C. The reaction was checked by LC-MS, solvent was removed and the residue was purified by silica gel column chromatography (EtOAc/hexane→CH2Cl2/MeOH). Pure fractions were combined and concentrated. The residue was dissolved in CH₃CN:H₂O (1:1, 2 mL) and lyophilized to obtain 1-(3-cyclopropyl-1-methyl-1H-pyrazol-5-yl)-3-(2-fluoro-4-(2-(1-methyl-1H-pyrazol-4-yl)pyridin-4-yloxy)phenyl)urea (26 mg, 13% yield). ¹H NMR (400 MHz, DMSO-d₆): δ 8.92 (s, 1H), 8.82 (d, J=2.0 Hz, 1H), 8.39 (d, J=6.0 Hz, 1H), 8.28 (s, 1H), 8.18 (t, J=9.6 Hz, 1H), 7.99 (s, 1H), 7.26 (m, 2H), 7.02 (m, 1H), 6.70 (dd, J=2.4, and 6.0 Hz, 1H), 3.87 (s, 3H), 3.59 (s, 3H), 1.76 (m, 1H), 0.80 (m, 2H), 0.59 (m, 2H); MS (ESI) m/z: 448.1 (M+H⁺).

### Example 34

Example B24 (100 mg, 0.333 mmol), Example A1 (95 mg, 0.333 mmol) and iPr₂NEt (0.127 ml, 0.732 mmol) were combined in DMSO (4 ml) and stirred with heating at 80° C. After 72 h, the crude reaction mixture was purified directly without aqueous workup by reverse phase chromatography to afford 1-(2-fluoro-4-(2-(1-methyl-1H-pyrazol-4-yl)pyridin-4-yloxy)phenyl)-3-(1-isopropyl-1H-imidazol-4-yOure a (110 mg, 60% yield) as the TFA salt. ¹H NMR (400 MHz, DMSO-d₆): δ 9.49 (s, 1H), 9.11 (brs, 1H), 8.50 (brs, 1H), 8.49 (d, 1H), 8.41 (s, 1H), 8.16-8.13 (m, 1H), 8.05 (s, 1H), 7.47-7.38 (brm, 2H), 7.37-7.31 (m, 1H), 7.09-7.05 (m, 1H), 6.92-6.87 (m, 1H), 4.55-4.46 (m, 1H), 3.88 (s, 3H), 1.44 (d, 6H); MS (ESI) m/z: 436.1 (M+H⁺).

### Example 35

Using General Method C, 1-tert-butyl-5-oxopyrrolidine-3-carboxylic acid (0.1 g, 0.54 mmol), Example A1 0.15 g, 0.54 mmol), Et3N (0.23 mL, 1.62 mmol) and DPPA (0.18 mL, 0.81 mmol)were combined and purified by silica gel column chromatography (EtOAc→CH₂Cl₂/MeOH) to obtain 1-(1-tert-butyl-5-oxopyrrolidin-3-yl)-3-(2-fluoro-4-(2-(1-methyl-1H-pyrazol-4-yl)pyridin-4-yloxy)phenyl)urea (0.13 g, 50% yield). ¹H NMR (400 MHz, DMSO-d₆): δ 8.35 (d, J=5.6 Hz, 1H), 8.29 (brs, 1H), 8.24 (s, 1H), 8.15 (t, J=9.2 Hz, 1H), 7.94 (s, 1H), 7.19 (m, 2H), 7.01 (d, J=6.8 Hz, 1H), 6.95 (m, 1H), 6.64 (m, 1H), 4.14 (m, 1H), 3.84 (s, 3H), 3.71 (m, 1H), 3.22 (dd, J=3.6, and 10.4 Hz, 1H), 2.60 (m, 1H), 2.07 (m, 1H), 1.32 (s, 9H); MS (ESI) m/z: 467.2 (M+H⁺).

### Example 36

To a stirring solution of 1-(1-tert-butyl-5-oxopyrrolidin-3-yl)-3-(2-fluoro-4-(2-(1-methyl-1H-pyrazol-4-yl)pyridin-4-yloxy)phenyl)urea from Example 35 (95 mg, 0.20 mmol) in dry THF (3 ml) at RT was added 1.0 M LAH/THF (0.81 ml, 0.82 mmol). The resulting mixture was stirred overnight at RT. It was carefully quenched by the sequential addition of H₂O (0.1 ml), 3M NaOH (0.1 ml) and H₂O (0.3 ml) and then EtOAc was added. The mixture was stirred at RT for 4 hours. The solution was filtered through a pad of Celite° and washing forward with EtOAc. The organic layer was dried (Na₂SO₄), concentrated in vacuo and purified via silica gel column chromatography (CH₂Cl₂/MeOH), dissolved in CH₃CN:H₂O (1:1 2 mL) and lyophilized to obtain 1-(1-tert-butylpyrrolidin-3-yl)-3-(2-fluoro-4-(2-(1-methyl-1H-pyrazol-4-yl)pyridin-4-yloxy)phenyl)urea (45 mg, 49% yield). ¹H NMR (400 MHz, DMSO-d₆): δ 8.42 (brs, 1H), 8.34 (d, J=6.0 Hz, 1H), 8.24 (s, 1H), 8.16 (t, J=8.8 Hz, 1H), 7.94 (s, 1H), 7.16 (m, 2H), 6.93 (m, 2H), 6.63 (dd, J=2.4, and 5.6 Hz, 1H), 4.05 (m, 1H), 3.84 (s, 3H), 2.3-2.8 (m, 4H), 2.03 (m, 1H), 1.48 (m, 1H), 1.01 (s, 9H); MS (ESI) m/z: 453.1 (M+H⁺).

### Example 37

Using a procedure analogous to Example 21, Example B25 (16 mg, 0.091 mmol), prop-1-en-2-yl2-fluoro-4-(2-(1-methyl-1H-pyrazol-4-yl)pyridin-4-yloxy)phenylcarbamate from Example 21 (35 mg, 0.095 mmol) and N-methylpyrrolidine (1 mg, 0.012 mmol) were combined in 1,4-dioxane (0.8 mL) at 60° C. to afford 1-(2-fluoro-4-(2-(1-methyl-1H-pyrazol-4-yl)pyridin-4-yloxy)phenyl)-3-(2-methyl-5-(trifluoromethyl)pyridin-3-yl)urea (28 mg, 63% yield). ¹H NMR (400 MHz, DMSO-d₆): δ 9.30 (s, 1H), 8.79 (s, 1H), 8.68 (s, 1H), 8.47 (s, 1H), 8.37 (d, J=5.6 Hz, 1H), 8.25 (s, 1H), 8.22 (t, J=9.4 Hz, 1H), 7.96 (s, 1H), 7.28 (dd, J=12.3, 1.9 Hz, 1H), 7.23 (s, 1 H), 7.02 (m, 1H), 6.67 (m, 1H), 3.84 (s, 3H), 2.57 (s, 3H); MS (ESI) m/z: 487.2 (M+H⁺).

### Example 38

Using General Method C, Example B23 (64 mg, 0.35 mmol), Example A1 (0.1 g, 0.35 mmol), Et₃N (54 μL, 0.38 mmol) DPPA (83 μL, 0.38 mmol) were combined and purified by reverse-phase column chromatography (CH₃CN/H₂O (0.1% TFA)) provide the TFA salt of 1-(1-tert-butyl-5-methyl-1H-pyrazol-3-yl)-3-(2-fluoro-4-(2-(1-methyl-1H-pyrazol-4-yl)pyridin-4-yloxy)phenyl)urea. The salt was treated with EtOAc and NaHCO₃ and then the solution was stirred at RT for 1 hour. The organic was separated, dried (Na₂SO₄), and titurated (Et2O) to obtain 1-(1-tert-butyl-5-methyl-1H-pyrazol-3-yl)-3-(2-fluoro-4-(2-(1-methyl-1H-pyrazol-4-yl)pyridin-4-yloxy)phenyl)urea (55 mg, 35% yield). ¹H NMR (400 MHz, DMSO-d₆): δ 9.38 (brs, 1H), 8.35 (m, 1H), 8.30 (m, 1H), 8.25 (s, 1H), 7.95 (m, 1H), 7.25 (dd, J=2.4, and 12.0 Hz, 1H), 7.20 (d, J=2.0 Hz, 1H), 7.00 (m, 1H), 6.67 (dd, J=2.4, and 5.6 Hz, 1H), 5.82 (brs, 1H), 3.84 (s, 3H), 2.36 (s, 3H), 1.54 (s, 9H); MS (ESI) m/z: 464.2 (M+H⁺).

### Example 40

Using General Method C, Example B26 (70 mg, 0.19 mmol) and Example A1 (55 mg, 0.19 mmol) in presence of DPPA (55 μL, 0.21 mmol) and (30 μL, 0.21 mmol) were combined and the resultant product purified via column chromatography (methanol/methylene chloride) to afford tert-butyl 4-(2-tert-butyl-5-(3-(2-fluoro-4-(2-(1-methyl-1H-pyrazol-4-yl)pyridin-4-yloxy)phenyl)ureido)pyrimidin-4-yl)piperazine-1-carboxylate. MS (ESI) m/z: 646.3 (M+H⁺). This was then treated with HCl (4.0 M, in dioxane) to afford tert-butyl 4-(2-tert-butyl-5-(3-(2-fluoro-4-(2-(1-methyl-1H-pyrazol-4-yl)pyridin-4-yloxy)phenyl)ureido)pyrimidin-4-yl)piperazine-1-carboxylate HCl salt (67 mg, 56% yield). ¹H NMR (400 MHz, DMSO-d₆): δ 9.51 (brs, 1H), 9.31 (brs, 2H), 8.68 (brs, 1H), 8.51 (m, 2H), 8.36 (brs, 1H), 8.20 (t, J=9.2 Hz, 1H), 7.65 (brs, 1H), 7.41 (brd, J=11.6 Hz, 1H), 7.12 (brd, J=9.6 Hz, 1H), 7.06 (brs, 1H), 3.95 (m, 4H), 3.90 (s, 3H), 3.26 (m, 4H), 1.35 (s, 9H); MS (ESI) m/z: 646.3 (M+H⁺).

### Example 41

Using General Method C, Example B27 (60 mg, 0.23 mmol) and Example A1 (64 mg, 0.23 mmol) in presence of DPPA (57 μL, 0.23 mmol) and (36 μL, 0.23 mmol) were combined and the resultant product purified via column chromatography (CH₂Cl₂/MeOH) to afford 1-(2-tert-butyl-4-morpholinopyrimidin-5-yl)-3-(2-fluoro-4-(2-(1-methyl-1H-pyrazol-4-yl)pyridin-4-yloxy)phenyl)urea (94 mg, 76% yield). ¹H NMR (400 MHz, DMSO-d₆): δ 8.95 (brs, 1H), 8.39 (s, 1H), 8.36 (d, J=5.6 Hz, 1H), 8.24 (m, 2H), 8.16 (t, J=9.6 Hz, 1H), 7.95 (s, 1H), 7.24 (dd, J=2.8, and 11.6 Hz, 1H), 7.21 (d, J=2.4 Hz, 1H), 7.00 (m, 1H), 6.66 (dd, J=2.4, and 6.0 Hz, 1H), 3.84 (s, 3H), 3.71 (m, 4H), 3.49 (m, 4H)m 1.29 (s, 9H); MS (ESI) m/z: 547.3 (M+H⁺).

### Example 42

A mixture of Example A1 (2.0 g, 7.04 mmol) and saturated aq NaHCO₃ (100 mL) in EtOAc (100 mL) was cooled in an ice bath and treated with isopropenyl chloroformate (1.6 mL, 14.64 mmol). The reaction mixture was allowed to slowly warm to RT overnight. The organic layer was separated and washed with sat aq NaHCO₃ (25 mL) and brine (25 mL), dried (MgSO₄), concentrated in vacuo and re-crystallized (diethylether) to provide prop-1-en-2-yl 2-fluoro-4-(2-(1-methyl-1H-pyrazol-4-yl)pyridin-4-yloxy)phenylcarbamate (2.32 g, 90% yield). ¹H NMR (400 MHz, DMSO-d₆): δ 9.69 (br s, 1H), 8.38 (d, J=5.6 Hz, 1H), 8.26 (s, 1H), 7.96 (d, J=0.8 Hz, 1H), 7.67 (br t, J=8.4 Hz, 1H), 7.27 (d, J=2.4 Hz, 1H), 7.22 (dd, J=11.2, 2.4 Hz, 1 H), 7.00 (m, 1H), 6.69 (dd, J=5.6, 2.4 Hz, 1H), 4.74 (m, 1H), 4.72 (s, 1H), 3.84 (s, 3 H), 1.92 (s, 3H); MS (ESI) m/z: 369.1 (M+H⁺).

### Example B28

(20 mg, 0.083 mmol), prop-1-en-2-yl2-fluoro-4-(2-(1-methyl-1H-pyrazol-4-yl)pyridin-4-yloxy)phenylcarbamate (30 mg, 0.083 mmol) and N-methylpyrrolidine (1 mg, 0.012 mmol) were combined in THF (1.5 mL) and heated to 55° C. in capped vial for 6 days. 1,8-Diazabicyclo[5.4.0]undece-7-ene (1 drop) was added and the mixture was heated for an additional 3 h at 55° C. The solvent was removed in vacuo and the residue was purifed by silica gel chromatography. A second reverse-phase chromatography provided 1-(2-fluoro-4-(2-(1-methyl-1H-pyrazol-4-yl)pyridin-4-yloxy)phenyl)-3-(2-(1-methyl-1H-pyrazol-4-yl)-5-(trifluoromethyl)pyridin-3-yl)urea (16 mg, 35% yield). ¹H NMR (400 MHz, Acetone-d₆): δ 9.15 (s, 1H), 8.81 (s, 1H), 8.61 (s, 1H), 8.59 (s, 1H), 8.40-8.31 (m, 3H), 8.13 (s, 1H), 8.04 (s, 1H), 7.94 (s, 1H), 7.19 (d, J=2.4 Hz, 1H), 7.09 (dd, J=11.6, 2.6 Hz, 1H), 7.02 (m, 1H), 6.71 (dd, J=5.6, 2.6 Hz, 1H), 3.97 (s, 3H), 3.91 (s, 3H); MS (ESI): m/z 553.2 (M+H⁺).

Using the synthetic procedures and methods described herein and methods known to those skilled in the art, the following compounds were made:
1-(3-tert-butylisoxazol-5-yl)-3-(3-methyl-4-(2-(1-methyl-1H-pyrazol-4-yl)pyridin-4-yloxy)phenyl)urea, 1-(3-tert-butyl-1-methyl-1H-pyrazol-5-yl)-3-(3-methyl-4-(2-(1-methyl-1H-pyrazol-4-yl)pyridin-4-yloxy)phenyl)urea, 1-(4-(2-(1-methyl-1H-pyrazol-4-yl)pyridin-4-yloxy)phenyl)-3-(3-(trifluoromethyl)phenyl)urea, 1-(5-tert-butylisoxazol-3-yl)-3-(4-(2-(1-methyl-1H-pyrazol-4-yl)pyridin-4-yloxy)phenyl)urea, 1-(4-chloro-3-(trifluoromethyl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(5-isopropylisoxazol-3-yl)urea, 1-(2,3-difluorophenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-isopropylisoxazol-5-yl)urea, 1-(3,5-dichlorophenyl)-3-(2-fluoro-4-(2-(1-methyl-1H-pyrazol-4-yl)pyridin-4-yloxy)phenyl)urea, 1-cyclohexyl-3-(2-fluoro-4-(2-(1-methyl-1H-pyrazol-4-yl)pyridin-4-yloxy)phenyl)urea, 1-cyclop entyl-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(1-isopropyl-1H-pyrazol-4-yl)urea, 1-(4-chlorophenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(1-methyl-3-(1-methylcyclopentyl)-1H-pyrazol-5-yl)urea, 1-(2-fluoro-4-(2-(1-methyl-1H-pyrazol-4-yl)pyridin-4-yloxy)phenyl)-3-(2-fluoro-5-(trifluoromethyl)phenyl)urea, 1-(3-tert-butylphenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(2-fluoro-5-methylphenyl)urea, 1-(2-fluoro-4-(2-(1-methyl-1H-pyrazol-4-yl)pyridin-4-yloxy)phenyl)-3-(3-isopropylphenyl)urea, 1-(1-tert-butyl-1H-pyrazol-4-yl)-3-(3-fluoro-4-(2-(1-methyl-1H-pyrazol-4-yl)pyridin-4-yloxy)phenyl)urea, 1-(5-fluoro-2-methylphenyl)-3-(2-fluoro-4-(2-(1-methyl-1H-pyrazol-4-yl)pyridin-4-yloxy)phenyl)urea, 1-(3-cyclop entyl-1-methyl-1H-pyrazol-5-yl)-3-(2-fluoro-4-(2-(1-methyl-1H-pyrazol-4-yl)pyridin-4-yloxy)phenyl)urea, 1-(1-tert-butyl-1H-pyrazol-4-yl)-3-(2-fluoro-4-(2-(1-propyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-fluorophenyl)urea, 1-(2-fluoro-3-methyl-4-(2-(1-methyl-1H-pyrazol-4-yl)pyridin-4-yloxy)phenyl)-3-(1-isopropyl-1H-pyrazol-4-yl)urea, 1-cyclohexyl-3-(2,3-difluoro-4-(2-(1-methyl-1H-pyrazol-4-yl)pyridin-4-yloxy)phenyl)urea, 1-cyclohexyl-3-(2-fluoro-3-methyl-4-(2-(1-methyl-1H-pyrazol-4-yl)pyridin-4-yloxy)phenyl)urea, 1-(1-cyclopentyl-5-methyl-1H-pyrazol-4-yl)-3-(2,3-difluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(5-fluoropyridin-3-yl)urea, 1-(3-cyanophenyl)-3-(2-fluoro-4-(2-(1-methyl-1H-pyrazol-4-yl)pyridin-4-yloxy)phenyl)urea, 1-(3-tert-butylisoxazol-5-yl)-3-(2-fluoro-3-methyl-4-(2-(1-methyl-1H-pyrazol-4-yl)pyridin-4-yloxy)phenyl)urea, 1-(3-tert-butylisoxazol-5-yl)-3-(3-fluoro-4-(2-(1-methyl-1H-pyrazol-4-yl)pyridin-4-yloxy)phenyl)urea, 1-(3-tert-butyl-1-methyl-1H-pyrazol-5-yl)-3-(2,3-difluoro-4-(2-(1-methyl-1H-pyrazol-4-yl)pyridin-4-yloxy)phenyl)urea, 1-(3-tert-butyl-1-methyl-1H-pyrazol-5-yl)-3-(2-fluoro-3-methyl-4-(2-(1-methyl-1H-pyrazol-4-yl)pyridin-4-yloxy)phenyl)urea, 1-(1-cyclopentyl-1H-pyrazol-4-yl)-3-(2,3-difluoro-4-(2-(1-methyl-1H-pyrazol-4-yl)pyridin-4-yloxy)phenyl)urea, 1-(2,3-difluoro-4-(2-(1-methyl-1H-pyrazol-4-yl)pyridin-4-yloxy)phenyl)-3-(3-isopropylisoxazol-5-yl)urea, 1-(2-fluoro-3-methyl-4-(2-(1-methyl-1H-pyrazol-4-yl)pyridin-4-yloxy)phenyl)-3-(3-isopropylisoxazol-5-yl)urea, 1-(2-fluoro-3-methyl-4-(2-(1-methyl-1H-pyrazol-4-yl)pyridin-4-yloxy)phenyl)-3-(6-fluorobenzo[d]thiazol-2-yl)urea, 1-(2-fluoro-3-methyl-4-(2-(1-methyl-1H-pyrazol-4-yl)pyridin-4-yloxy)phenyl)-3-(5-isopropylpyridin-3-yl)urea, 1-(2,3-difluoro-4-(2-(1-methyl-1H-pyrazol-4-yl)pyridin-4-yloxy)phenyl)-3-(5-isopropylpyridin-3-yl)urea, 1-(2-fluoro-4-(2-(1-methyl-1H-pyrazol-4-yl)pyridin-4-yloxy)phenyl)-3-(5-methylpyridin-3-yl)urea, 1-(2-fluoro-3-methyl-4-(2-(1-methyl-1H-pyrazol-4-yl)pyridin-4-yloxy)phenyl)-3-(5-(trifluoromethyl)pyridin-3-yl)urea, 1-(2,3-difluoro-4-(2-(1-methyl-1H-pyrazol-4-yl)pyridin-4-yloxy)phenyl)-3-(5-(trifluoromethyl)pyridin-3-yl)urea, 1-(5-chloropyridin-3-yl)-3-(2-fluoro-4-(2-(1-methyl-1H-pyrazol-4-yl)pyridin-4-yloxy)phenyl)urea, and 1-(2-fluoro-4-(2-(1-methyl-1H-pyrazol-4-yl)pyridin-4-yloxy)phenyl)-3-(3-isopropyl-1-methyl-1H-pyrazol-5-yl)urea.

Using the synthetic procedures and methods described herein and methods known to those skilled in the art, the following compounds are made:
1-(3-tert-butylisoxazol-5-yl)-3-(2-fluoro-4-(2-(1-methyl-1H-pyrazol-3-yl)pyridin-4-yloxy)phenyl)urea, 1-(3-tert-butylisoxazol-5-yl)-3-(2-fluoro-4-(2-(1-methyl-1H-pyrazol-5-yl)pyridin-4-yloxy)phenyl)urea, 1-(3-tert-butyl-1-methyl-1H-pyrazol-5-yl)-3-(2-fluoro-4-(2-(1-methyl-1H-pyrazol-3-yl)pyridin-4-yloxy)phenyl)urea, 1-(3-tert-butyl-1-methyl-1H-pyrazol-5-yl)-3-(2-fluoro-4-(2-(1-methyl-1H-pyrazol-5-yl)pyridin-4-yloxy)phenyl)urea, 1-(3-tert-butyl-1-methyl-1H-pyrazol-5-yl)-3-(2-fluoro-4-(2-(3-methyl-1H-pyrazol-1-yl)pyridin-4-yloxy)phenyl)urea, 1-(2-fluoro-4-(2-(1-methyl-1H-pyrazol-4-yl)pyridin-4-yloxy)phenyl)-3-(3-(1-hydroxy-2-methylpropan-2-yl)-1-methyl-1H-pyrazol-5-yl)urea, 1-(2-fluoro-4-(2-(1-methyl-1H-pyrazol-4-yl)pyridin-4-yloxy)phenyl)-3-(3-(1-hydroxy-2-methylpropan-2-yl)isoxazol-5-yl)urea, 1-(2-fluoro-4-(2-(1-methyl-1H-pyrazol-4-yl)pyridin-4-yloxy)phenyl)-3-(5-(2-hydroxypropan-2-yl)pyridin-3-yl)urea, 1-(3-tert-butyl-1-methyl-1H-pyrazol-5-yl)-3-(2-fluoro-4-(2-(1-(2-hydroxyethyl)-1H-pyrazol-4-yl)pyridin-4-yloxy)phenyl)urea, 1-(3-tert-butyl-1-methyl-1H-pyrazol-5-yl)-3-(4-(2-(1-(2-(dimethylamino)ethyl)-1H-pyrazol-4-yl)pyridin-4-yloxy)-2-fluorophenyl)urea, 1-(3-tert-butyl-1-methyl-1H-pyrazol-5-yl)-3-(4-(2-(1-(cyanomethyl)-1H-pyrazol-4-yl)pyridin-4-yloxy)-2-fluorophenyl)urea, 1-(4-(2-(1-(2-amino-2-oxo ethyl)-1H-pyrazol-4-yl)pyridin-4-yloxy)-2,3-difluorophenyl)-3-(5-isopropylpyridin-3-yl)urea, 1-(4-(2-(1-(cyanomethyl)-1H-pyrazol-4-yl)pyridin-4-yloxy)-2,3-difluorophenyl)-3-(5-isopropylpyridin-3-yl)urea, 1-(2,3-difluoro-4-(2-(1-(2-morpholino ethyl)-1H-pyrazol-4-yl)pyridin-4-yloxy)phenyl)-3-(5-isopropylpyridin-3-yl)urea, 1-(2-fluoro-4-(2-(1-(2-morpholinoethyl)-1H-pyrazol-4-yl)pyridin-4-yloxy)phenyl)-3-(5-isopropylpyridin-3-yl)urea, 1-(2-fluoro-4-(2-(1-propyl-1H-pyrazol-4-yl)pyridin-4-yloxy)phenyl)-3-(5-isopropylpyridin-3-yl)urea, 1-(2,3-difluoro-4-(2-(1-(2-methoxyethyl)-1H-pyrazol-4-yl)pyridin-4-yloxy)phenyl)-3-(5-isopropylpyridin-3-yl)urea 1-(2,3-difluoro-4-(2-(1-(2-hydroxyethyl)-1H-pyrazol-4-yl)pyridin-4-yloxy)phenyl)-3-(5-isopropylpyridin-3-yl)urea, 1-(4-(2-(1-(2-(dimethylamino)ethyl)-1H-pyrazol-4-yl)pyridin-4-yloxy)-2,3-difluorophenyl)-3-(5-isopropylpyridin-3-yl)urea, 1-(4-(2-(1-(3-(dimethylamino)propyl)-1H-pyrazol-4-yl)pyridin-4-yloxy)-2,3-difluorophenyl)-3-(5-isopropylpyridin-3-yl)urea, 1-(2,3-difluoro-4-(2-(1-(3-hydroxypropyl)-1H-pyrazol-4-yl)pyridin-4-yloxy)phenyl)-3-(5-isopropylpyridin-3-yl)urea, 1-(4-(2-(1-(2-(dimethylamino)ethyl)-1H-pyrazol-4-yl)pyridin-4-yloxy)-2-fluorophenyl)-3-(5-isopropylpyridin-3-yl)urea, 1-(2-fluoro-4-(2-(1-methyl-1H-pyrazol-4-yl)pyridin-4-yloxy)phenyl)-3-(4-(trifluoromethyl)pyridin-2-yl)urea, 1-(3-fluoro-4-(2-(1-(2-(4-methylpiperazin-1-yl)ethyl)-1H-pyrazol-4-yl)pyridin-4-yloxy)phenyl)-3-(5-isopropylpyridin-3-yl)urea, 1-(2-fluoro-4-(2-(1-(3-hydroxypropyl)-1H-pyrazol-4-yl)pyridin-4-yloxy)phenyl)-3-(5-isopropylpyridin-3-yl)urea, 1-(2-fluoro-4-(2-(1-methyl-1H-pyrazol-4-yl)pyridin-4-yloxy)phenyl)-3-(4-(trifluoromethyl)pyridin-2-yl)urea, 1-(3-tert-butyl-1-methyl-1H-pyrazol-5-yl)-3-(4-(2-(1-(2-(dimethylamino)ethyl)-1H-pyrazol-4-yl)pyridin-4-yloxy)-2,3-difluorophenyl)urea, 1-(3-tert-butyl-1-methyl-1H-pyrazol-5-yl)-3-(2,3-difluoro-4-(2-(1-(2-methoxyethyl)-1H-pyrazol-4-yl)pyridin-4-yloxy)phenyl)urea 1-(3-tert-butyl-1-methyl-1H-pyrazol-5-yl)-3-(2,3-difluoro-4-(2-(1-(2-hydroxyethyl)-1H-pyrazol-4-yl)pyridin-4-yloxy)phenyl)urea, 1-(3-tert-butyl-1-methyl-1H-pyrazol-5-yl)-3-(3-fluoro-4-(2-(1-(2-(4-methylpiperazin-1-yl)ethyl)-1H-pyrazol-4-yl)pyridin-4-yloxy)phenyl)urea, 1-(3-tert-butyl-1-methyl-1H-pyrazol-5-yl)-3-(4-(2-(1-(3-(dimethylamino)propyl)-1H-pyrazol-4-yl)pyridin-4-yloxy)-2,3-difluorophenyl)urea, 1-(3-tert-butyl-1-methyl-1H-pyrazol-5-yl)-3-(2,3-difluoro-4-(2-(1-(3-hydroxypropyl)-1H-pyrazol-4-yl)pyridin-4-yloxy)phenyl)urea, 1-(5-tert-butylpyridin-3-yl)-3-(2-fluoro-4-(2-(1-(3-hydroxypropyl)-1H-pyrazol-4-yl)pyridin-4-yloxy)phenyl)urea, 1-(5-tert-butylpyridin-3-yl)-3-(2-fluoro-4-(2-(1-methyl-1H-pyrazol-4-yl)pyridin-4-yloxy)phenyl)urea, 1-(3-tert-butyl-1-methyl-1H-pyrazol-5-yl)-3-(2-fluoro-4-(2-(1-methyl-1H-pyrazol-3-yl)pyridin-4-yloxy)phenyl)urea, 1-(3-tert-butyl-1-methyl-1H-pyrazol-5-yl)-3-(2-fluoro-4-(2-(1-methyl-1H-pyrazol-5-yl)pyridin-4-yloxy)phenyl)urea, 1-(3-tert-butylisoxazol-5-yl)-3-(2-fluoro-4-(2-(1-methyl-1H-pyrazol-3-yl)pyridin-4-yloxy)phenyl)urea, 1-(3-tert-butylisoxazol-5-yl)-3-(2-fluoro-4-(2-(1-methyl-1H-pyrazol-5-yl)pyridin-4-yloxy)phenyl)urea, 1-(3-tert-butyl-1-methyl-1H-pyrazol-5-yl)-3-(2-fluoro-4-(2-(3-methyl-1H-pyrazol-1-yl)pyridin-4-yloxy)phenyl)urea, 1-(2,3-difluoro-4-(2-(1-methyl-1H-pyrazol-4-yl)pyridin-4-yloxy)phenyl)-3-(3-(1-hydroxy-2-methylpropan-2-yl)-1-methyl-1H-pyrazol-5-yl)urea, 1-(2-fluoro-3-methyl-4-(2-(1-methyl-1H-pyrazol-4-yl)pyridin-4-yloxy)phenyl)-3-(3-(1-hydroxy-2-methylpropan-2-yl)-1-methyl-1H-pyrazol-5-yl)urea, 1-(2-fluoro-3-methyl-4-(2-(1-methyl-1H-pyrazol-4-yl)pyridin-4-yloxy)phenyl)-3-(3-(1-hydroxy-2-methylpropan-2-yl)isoxazol-5-yl)urea, 1-(2,3-difluoro-4-(2-(1-methyl-1H-pyrazol-4-yl)pyridin-4-yloxy)phenyl)-3-(3-(1-hydroxy-2-methylpropan-2-yl)isoxazol-5-yl)urea, 1-(2-fluoro-4-(2-(1-methyl-1H-pyrazol-4-yl)pyridin-4-yloxy)phenyl)-3-(5-(1-hydroxy-2-methylpropan-2-yl)pyridin-3-yl)urea, 1-(2-fluoro-3-methyl-4-(2-(1-methyl-1H-pyrazol-4-yl)pyridin-4-yloxy)phenyl)-3-(5-(1-hydroxy-2-methylpropan-2-yl)pyridin-3-yl)urea, 1-(2,3-difluoro-4-(2-(1-methyl-1H-pyrazol-4-yl)pyridin-4-yloxy)phenyl)-3-(5-(1-hydroxy-2-methylpropan-2-yl)pyridin-3-yl)urea, 1-(2-fluoro-4-(2-(1-methyl-1H-pyrazol-4-yl)pyridin-4-yloxy)phenyl)-3-(5-(1-hydroxypropan-2-yl)pyridin-3-yl)urea, 1-(2-fluoro-3-methyl-4-(2-(1-methyl-1H-pyrazol-4-yl)pyridin-4-yloxy)phenyl)-3-(5-(1-hydroxypropan-2-yl)pyridin-3-yl)urea, 1-(2,3-difluoro-4-(2-(1-methyl-1H-pyrazol-4-yl)pyridin-4-yloxy)phenyl)-3-(5-(1-hydroxypropan-2-yl)pyridin-3-yl)urea, 1-(2,3-difluoro-4-(2-(1-methyl-1H-pyrazol-4-yl)pyridin-4-yloxy)phenyl)-3-(5-ethylpyridin-3-yl)urea, 1-(5-ethylpyridin-3-yl)-3-(2-fluoro-3-methyl-4-(2-(1-methyl-1H-pyrazol-4-yl)pyridin-4-yloxy)phenyl)urea, 1-(1-tert-butyl-1H-pyrazol-3-yl)-3-(2-fluoro-4-(2-(1-methyl-1H-pyrazol-4-yl)pyridin-4-yloxy)phenyl)urea, 1-(3-tert-butyl-1-methyl-1H-pyrazol-5-yl)-3-(4-(2-(1-ethyl-1H-pyrazol-4-yl)pyridin-4-yloxy)-2-fluorophenyl)urea, 1-(4-(2-(1-(2-amino-2-oxoethyl)-1H-pyrazol-4-yl)pyridin-4-yloxy)-2-fluorophenyl)-3-(3-tert-butyl-1-methyl-1H-pyrazol-5-yl)urea, 1-(3-tert-butyl-1-methyl-1H-pyrazol-5-yl)-3-(2-fluoro-4-(2-(1-(2-morpholino ethyl)-1H-pyrazol-4-yl)pyridin-4-yloxy)phenyl)urea, 1-(3-tert-butylisoxazol-5-yl)-3-(4-(2-(1-ethyl-1H-pyrazol-4-yl)pyridin-4-yloxy)-2-fluorophenyl)urea, 1-(3-tert-butylisoxazol-5-yl)-3-(4-(2-(1-(cyanomethyl)-1H-pyrazol-4-yl)pyridin-4-yloxy)-2-fluorophenyl)urea, 1-(4-(2-(1-(2-amino-2-oxoethyl)-1H-pyrazol-4-yl)pyridin-4-yloxy)-2-fluorophenyl)-3-(3-tert-butylisoxazol-5-yl)urea, 1-(3-tert-butylisoxazol-5-yl)-3-(2-fluoro-4-(2-(1-(2-morpholino ethyl)-1H-pyrazol-4-yl)pyridin-4-yloxy)phenyl)urea, 1-(3-tert-butylisoxazol-5-yl)-3-(2-fluoro-4-(2-(1-(2-hydroxyethyl)-1H-pyrazol-4-yl)pyridin-4-yloxy)phenyl)urea, 1-(3-tert-butylisoxazol-5-yl)-3-(4-(2-(1-(2-(dimethylamino)ethyl)-1H-pyrazol-4-yl)pyridin-4-yloxy)-2-fluorophenyl)urea, 1-(1-tert-butyl-1H-pyrazol-4-yl)-3-(4-(2-(1-ethyl-1H-pyrazol-4-yl)pyridin-4-yloxy)-2-fluorophenyl)urea, 1-(1-tert-butyl-1H-pyrazol-4-yl)-3-(4-(2-(1-(cyanomethyl)-1H-pyrazol-4-yl)pyridin-4-yloxy)-2-fluorophenyl)urea, 1-(4-(2-(1-(2-amino-2-oxo ethyl)-1H-pyrazol-4-yl)pyridin-4-yloxy)-2-fluorophenyl)-3-(1-tert-butyl-1H-pyrazol-4-yl)urea, 1-(1-tert-butyl-1H-pyrazol-4-yl)-3-(4-(2-(1-(2-(dimethylamino)ethyl)-1H-pyrazol-4-yl)pyridin-4-yloxy)-2-fluorophenyl)urea, 1-(1-tert-butyl-1H-pyrazol-4-yl)-3-(2-fluoro-4-(2-(1-(2-hydroxyethyl)-1H-pyrazol-4-yl)pyridin-4-yloxy)phenyl)urea, 1-(1-tert-butyl-1H-pyrazol-4-yl)-3-(2-fluoro-4-(2-(1-(2-morpholinoethyl)-1H-pyrazol-4-yl)pyridin-4-yloxy)phenyl)urea, 1-(1-tert-butyl-1H-pyrazol-3-yl)-3-(4-(2-(1-ethyl-1H-pyrazol-4-yl)pyridin-4-yloxy)-2-fluorophenyl)urea, 1-(1-tert-butyl-1H-pyrazol-3-yl)-3-(4-(2-(1-(cyanomethyl)-1H-pyrazol-4-yl)pyridin-4-yloxy)-2-fluorophenyl)urea, 1-(4-(2-(1-(2-amino-2-oxoethyl)-1H-pyrazol-4-yl)pyridin-4-yloxy)-2-fluorophenyl)-3-(1-tert-butyl-1H-pyrazol-3-yl)urea, 1-(1-tert-butyl-1H-pyrazol-3-yl)-3-(4-(2-(1-(2-(dimethylamino)ethyl)-1H-pyrazol-4-yl)pyridin-4-yloxy)-2-fluorophenyl)urea, 1-(1-tert-butyl-1H-pyrazol-3-yl)-3-(2-fluoro-4-(2-(1-(2-hydroxyethyl)-1H-pyrazol-4-yl)pyridin-4-yloxy)phenyl)urea, 1-(1-tert-butyl-1H-pyrazol-3-yl)-3-(2-fluoro-4-(2-(1-(2-morpholino ethyl)-1H-pyrazol-4-yl)pyridin-4-yloxy)phenyl)urea, 1-(4-(2-(1-ethyl-1H-pyrazol-4-yl)pyridin-4-yloxy)-2-fluorophenyl)-3-(5-isopropylpyridin-3-yl)urea, 1-(4-(2-(1-(cyanomethyl)-1H-pyrazol-4-yl)pyridin-4-yloxy)-2-fluorophenyl)-3-(5-isopropylpyridin-3-yl)urea, 1-(4-(2-(1-(2-amino-2-oxo ethyl)-1H-pyrazol-4-yl)pyridin-4-yloxy)-2-fluorophenyl)-3-(5-isopropylpyridin-3-yl)urea, 1-(4-(2-(1-(2-(dimethylamino)ethyl)-1H-pyrazol-4-yl)pyridin-4-yloxy)-2-fluorophenyl)-3-(5-isopropylpyridin-3-yl)urea, 1-(2-fluoro-4-(2-(1-(2-hydroxyethyl)-1H-pyrazol-4-yl)pyridin-4-yloxy)phenyl)-3-(5-isopropylpyridin-3-yl)urea, 1-(2-fluoro-4-(2-(1-(2-morpholino ethyl)-1H-pyrazol-4-yl)pyridin-4-yloxy)phenyl)-3-(5-isopropylpyridin-3-yl)urea, 1-(5-tert-butylpyridin-3-yl)-3-(4-(2-(1-ethyl-1H-pyrazol-4-yl)pyridin-4-yloxy)-2-fluorophenyl)urea, 1-(5-tert-butylpyridin-3-yl)-3-(4-(2-(1-(cyanomethyl)-1H-pyrazol-4-yl)pyridin-4-yloxy)-2-fluorophenyl)urea, 1-(4-(2-(1-(2-amino-2-oxoethyl)-1H-pyrazol-4-yl)pyridin-4-yloxy)-2-fluorophenyl)-3-(5-tert-butylpyridin-3-yl)urea, 1-(5-tert-butylpyridin-3-yl)-3-(4-(2-(1-(2-(dimethylamino)ethyl)-1H-pyrazol-4-yl)pyridin-4-yloxy)-2-fluorophenyl)urea, 1-(5-tert-butylpyridin-3-yl)-3-(2-fluoro-4-(2-(1-(2-hydroxyethyl)-1H-pyrazol-4-yl)pyridin-4-yloxy)phenyl)urea, 1-(5-tert-butylpyridin-3-yl)-3-(2-fluoro-4-(2-(1-(2-morpholino ethyl)-1H-pyrazol-4-yl)pyridin-4-ylo xy)phenyl)urea, 1-(4-(2-(1-ethyl-1H-pyrazol-4-yl)pyridin-4-yloxy)-2-fluorophenyl)-3-(5-(trifluoromethyl)pyridin-3-yl)urea, 1-(4-(2-(1-(cyanomethyl)-1H-pyrazol-4-yl)pyridin-4-yloxy)-2-fluorophenyl)-3-(5-(trifluoromethyl)pyridin-3-yl)urea, 1-(4-(2-(1-(2-amino-2-oxoethyl)-1H-pyrazol-4-yl)pyridin-4-yloxy)-2-fluorophenyl)-3-(5-(trifluoromethyl)pyridin-3-yl)urea, 1-(4-(2-(1-(2-(dimethylamino)ethyl)-1H-pyrazol-4-yl)pyridin-4-yloxy)-2-fluorophenyl)-3-(5-(trifluoromethyl)pyridin-3-yl)urea, 1-(2-fluoro-4-(2-(1-(2-hydroxyethyl)-1H-pyrazol-4-yl)pyridin-4-yloxy)phenyl)-3-(5-(trifluoromethyl)pyridin-3-yl)urea, 1-(2-fluoro-4-(2-(1-(2-morpholinoethyl)-1H-pyrazol-4-yl)pyridin-4-ylo xy)phenyl)-3-(5-(trifluoromethyl)pyridin-3-yl)urea, 1-(2-fluoro-5-(2-(1-methyl-1H-pyrazol-4-yl)pyridin-4-yloxy)phenyl)-3-(5-(trifluoromethyl)pyridin-3-yl)urea, 1-(2-fluoro-5-(2-(1-methyl-1H-pyrazol-4-yl)pyridin-4-yloxy)phenyl)-3-(5-isopropylpyridin-3-yl)urea, 1-(1-tert-butyl-1H-pyrazol-4-yl)-3-(2-fluoro-5-(2-(1-methyl-1H-pyrazol-4-yl)pyridin-4-yloxy)phenyl)urea, 1-(1-tert-butyl-1H-pyrazol-3-yl)-3-(2-fluoro-5-(2-(1-methyl-1H-pyrazol-4-yl)pyridin-4-yloxy)phenyl)urea, 1-(3-tert-butylisoxazol-5-yl)-3-(2-fluoro-5-(2-(1-methyl-1H-pyrazol-4-yl)pyridin-4-yloxy)phenyl)urea, 1-(3-tert-butyl-1-methyl-1H-pyrazol-5-yl)-3-(2-fluoro-5-(2-(1-methyl-1H-pyrazol-4-yl)pyridin-4-yloxy)phenyl)urea, 1-(5-(6-(1H-pyrazol-4-yl)pyridin-2-yloxy)-2-fluorophenyl)-3-(3-(3-(dimethylamino)pyrrolidin-1-yl)phenyl)urea, 1-(5-(6-(1H-pyrazol-4-yl)pyridin-2-yloxy)-2-fluorophenyl)-3-(3-chloro-5-(3-(dimethylamino)pyrrolidin-1-yl)phenyl)urea, 1-(5-(6-(1H-pyrazol-4-yl)pyridin-2-yloxy)-2-fluorophenyl)-3-(3-(3-(dimethylamino)pyrrolidin-1-yl)-5-methylphenyl)urea, 1-(5-(6-(1H-pyrazol-4-yl)pyridin-2-yloxy)-2-fluorophenyl)-3-(3-(3-oxopyrrolidin-1-yl)phenyl)urea, 1-(5-(6-(1H-pyrazol-4-yl)pyridin-2-yloxy)-2-fluorophenyl)-3-(3-methyl-5-(3-oxopyrrolidin-1-yl)phenyl)urea, 1-(5-(6-(1H-pyrazol-4-yl)pyridin-2-yloxy)-2-fluorophenyl)-3-(3-chloro-5-(3-oxopyrrolidin-1-yl)phenyl)urea, 1-(5-(6-(1H-pyrazol-4-yl)pyridin-2-yloxy)-2-fluorophenyl)-3-(3-(3-oxopyrrolidin-1-yl)-5-(trifluoromethyl)phenyl)urea, 1-(3-(3-(dimethylamino)pyrrolidin-1-yl)phenyl)-3-(2-fluoro-5-(6-(1-methyl-1H-pyrazol-4-yl)pyridin-2-yloxy)phenyl)urea, 1-(3-chloro-5-(3-(dimethylamino)pyrrolidin-1-yl)phenyl)-3-(2-fluoro-5-(6-(1-methyl-1H-pyrazol-4-yl)pyridin-2-yloxy)phenyl)urea, 1-(3-(3-(dimethylamino)pyrrolidin-1-yl)-5-methylphenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-(3-oxopyrrolidin-1-yl)phenyl)urea, 1-(2-fluoro-5-(6-(1-methyl-1H-pyrazol-4-yl)pyridin-2-yloxy)phenyl)-3-(3-methyl-5-(3-oxopyrrolidin-1-yl)phenyl)urea, 1-(3-chloro-5-(3-oxopyrrolidin-1-yl)phenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-(3-oxopyrrolidin-1-yl)-5-(trifluoromethyl)phenyl)urea, 1-(5-(2-(1H-pyrazol-4-yl)pyridin-4-yloxy)-2-fluorophenyl)-3-(3-(3-(dimethylamino)pyrrolidin-1-yl)phenyl)urea, 1-(5-(2-(1H-pyrazol-4-yl)pyridin-4-yloxy)-2-fluorophenyl)-3-(3-chloro-5-(3-(dimethylamino)pyrrolidin-1-yl)phenyl)urea, 1-(5-(2-(1H-pyrazol-4-yl)pyridin-4-yloxy)-2-fluorophenyl)-3-(3-(3-(dimethylamino)pyrrolidin-1-yl)-5-methylphenyl)urea, 1-(5-(2-(1H-pyrazol-4-yl)pyridin-4-yloxy)-2-fluorophenyl)-3-(3-(3-oxopyrrolidin-1-yl)phenyl)urea, 1-(5-(2-(1H-pyrazol-4-yl)pyridin-4-yloxy)-2-fluorophenyl)-3-(3-methyl-5-(3-oxopyrrolidin-1-yl)phenyl)urea, 1-(5-(2-(1H-pyrazol-4-yl)pyridin-4-yloxy)-2-fluorophenyl)-3-(3-chloro-5-(3-oxopyrrolidin-1-yl)phenyl)urea, 1-(5-(2-(1H-pyrazol-4-yl)pyridin-4-yloxy)-2-fluorophenyl)-3-(3-(3-oxopyrrolidin-1-yl)-5-(trifluoromethyl)phenyl)urea, 1-(3-(3-(dimethylamino)pyrrolidin-1-yl)phenyl)-3-(2-fluoro-5-(2-(1-methyl-1H-pyrazol-4-yl)pyridin-4-yloxy)phenyl)urea, 1-(3-chloro-5-(3-(dimethylamino)pyrrolidin-1-yl)phenyl)-3-(2-fluoro-5-(2-(1-methyl-1H-pyrazol-4-yl)pyridin-4-yloxy)phenyl)urea, 1-(3-(3-(dimethylamino)pyrrolidin-1-yl)-5-methylphenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-(3-oxopyrrolidin-1-yl)phenyl)urea, 1-(2-fluoro-5-(2-(1-methyl-1H-pyrazol-4-yl)pyridin-4-yloxy)phenyl)-3-(3-methyl-5-(3-oxopyrrolidin-1-yl)phenyl)urea, 1-(3-chloro-5-(3-oxopyrrolidin-1-yl)phenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-(3-oxopyrrolidin-1-yl)-5-(trifluoromethyl)phenyl)urea, 1-(4-(2-(1H-pyrazol-4-yl)pyridin-4-yloxy)-2-fluorophenyl)-3-(3-(3-(dimethylamino)pyrrolidin-1-yl)phenyl)urea, 1-(4-(2-(1H-pyrazol-4-yl)pyridin-4-yloxy)-2-fluorophenyl)-3-(3-chloro-5-(3-(dimethylamino)pyrrolidin-1-yl)phenyl)urea, 1-(4-(2-(1H-pyrazol-4-yl)pyridin-4-yloxy)-2-fluorophenyl)-3-(3-(3-(dimethylamino)pyrrolidin-1-yl)-5-methylphenyl)urea, 1-(4-(2-(1H-pyrazol-4-yl)pyridin-4-yloxy)-2-fluorophenyl)-3-(3-(3-(dimethylamino)pyrrolidin-1-yl)-5-(trifluoromethyl)phenyl)urea, 1-(4-(2-(1H-pyrazol-4-yl)pyridin-4-yloxy)-2-fluorophenyl)-3-(3-(3-oxopyrrolidin-1-yl)phenyl)urea, 1-(4-(2-(1H-pyrazol-4-yl)pyridin-4-yloxy)-2-fluorophenyl)-3-(3-chloro-5-(3-oxopyrrolidin-1-yl)phenyl)urea, 1-(4-(2-(1H-pyrazol-4-yl)pyridin-4-yloxy)-2-fluorophenyl)-3-(3-methyl-5-(3-oxopyrrolidin-1-yl)phenyl)urea, 1-(4-(2-(1H-pyrazol-4-yl)pyridin-4-yloxy)-2-fluorophenyl)-3-(3-(3-oxopyrrolidin-1-yl)-5-(trifluoromethyl)phenyl)urea, 1-(3-(3-(dimethylamino)pyrrolidin-1-yl)phenyl)-3-(2-fluoro-4-(2-(1-methyl-1H-pyrazol-4-yl)pyridin-4-yloxy)phenyl)urea, 1-(3-chloro-5-(3-(dimethylamino)pyrrolidin-1-yl)phenyl)-3-(2-fluoro-4-(2-(1-methyl-1H-pyrazol-4-yl)pyridin-4-yloxy)phenyl)urea, 1-(3-(3-(dimethylamino)pyrrolidin-1-yl)-5-methylphenyl)-3-(2-fluoro-4-(2-(1-methyl-1H-pyrazol-4-yl)pyridin-4-yloxy)phenyl)urea, 1-(3-(3-(dimethylamino)pyrrolidin-1-yl)-5-(trifluoromethyl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-(3-oxopyrrolidin-1-yl)phenyl)urea, 1-(3-chloro-5-(3-oxopyrrolidin-1-yl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-(3-oxopyrrolidin-1-yl)phenyl)urea, 1-(2-fluoro-4-(2-(1-methyl-1H-pyrazol-4-yl)pyridin-4-yloxy)phenyl)-3-(3-(3-oxopyrrolidin-1-yl)-5-(trifluoromethyl)phenyl)urea, 1-(4-(2-(1H-pyrazol-4-yl)pyridin-4-yloxy)-3-fluorophenyl)-3-(3-(3-(dimethylamino)pyrrolidin-1-yl)phenyl)urea, 1-(4-(2-(1H-pyrazol-4-yl)pyridin-4-yloxy)-3-fluorophenyl)-3-(3-chloro-5-(3-(dimethylamino)pyrrolidin-1-yl)phenyl)urea, 1-(4-(2-(1H-pyrazol-4-yl)pyridin-4-yloxy)-3-fluorophenyl)-3-(3-(3-(dimethylamino)pyrrolidin-1-yl)-5-methylphenyl)urea, 1-(4-(2-(1H-pyrazol-4-yl)pyridin-4-yloxy)-3-fluorophenyl)-3-(3-(3-(dimethylamino)pyrrolidin-1-yl)-5-(trifluoromethyl)phenyl)urea, 1-(4-(2-(1H-pyrazol-4-yl)pyridin-4-yloxy)-3-fluorophenyl)-3-(3-(3-oxopyrrolidin-1-yl)phenyl)urea, 1-(4-(2-(1H-pyrazol-4-yl)pyridin-4-yloxy)-3-fluorophenyl)-3-(3-chloro-5-(3-oxopyrrolidin-1-yl)phenyl)urea, 1-(4-(2-(1H-pyrazol-4-yl)pyridin-4-yloxy)-3-fluorophenyl)-3-(3-methyl-5-(3-oxopyrrolidin-1-yl)phenyl)urea, 1-(4-(2-(1H-pyrazol-4-yl)pyridin-4-yloxy)-3-fluorophenyl)-3-(3-(3-oxopyrrolidin-1-yl)-5-(trifluoromethyl)phenyl)urea, 1-(3-(3-(dimethylamino)pyrrolidin-1-yl)phenyl)-3-(3-fluoro-4-(2-(1-methyl-1H-pyrazol-4-yl)pyridin-4-yloxy)phenyl)urea, 1-(3-chloro-5-(3-(dimethylamino)pyrrolidin-1-yl)phenyl)-3-(3-fluoro-4-(2-(1-methyl-1H-pyrazol-4-yl)pyridin-4-yloxy)phenyl)urea, 1-(3-(3-(dimethylamino)pyrrolidin-1-yl)-5-methylphenyl)-3-(3-fluoro-4-(2-(1-methyl-1H-pyrazol-4-yl)pyridin-4-yloxy)phenyl)urea, 1-(3-(3-(dimethylamino)pyrrolidin-1-yl)-5-(trifluoromethyl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-(3-oxopyrrolidin-1-yl)phenyl)urea, 1-(3-chloro-5-(3-oxopyrrolidin-1-yl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-(3-oxopyrrolidin-1-yl)phenyl)urea, 1-(3-fluoro-4-(2-(1-methyl-1H-pyrazol-4-yl)pyridin-4-yloxy)phenyl)-3-(3-(3-oxopyrrolidin-1-yl)-5-(trifluoromethyl)phenyl)urea, 1-(5-(6-(1H-pyrazol-4-yl)pyridin-2-yloxy)-2-fluorophenyl)-3-(3-(pyrrolidin-1-yl)phenyl)urea, 1-(5-(6-(1H-pyrazol-4-yl)pyridin-2-yloxy)-2-fluorophenyl)-3-(3-chloro-5-(pyrrolidin-1-yl)phenyl)urea, 1-(5-(6-(1H-pyrazol-4-yl)pyridin-2-yloxy)-2-fluorophenyl)-3-(3-methyl-5-(pyrrolidin-1-yl)phenyl)urea, 1-(5-(6-(1H-pyrazol-4-yl)pyridin-2-yloxy)-2-fluorophenyl)-3-(3-(pyrrolidin-1-yl)-5-(trifluoromethyl)phenyl)urea, 1-(5-(6-(1H-pyrazol-4-yl)pyridin-2-yloxy)-2-fluorophenyl)-3-(3-(4-methyl-1H-imidazol-1-yl)phenyl)urea, 1-(5-(6-(1H-pyrazol-4-yl)pyridin-2-yloxy)-2-fluorophenyl)-3-(3-chloro-5-(4-methyl-1H-imidazol-1-yl)phenyl)urea, 1-(5-(6-(1H-pyrazol-4-yl)pyridin-2-yloxy)-2-fluorophenyl)-3-(3-methyl-5-(4-methyl-1H-imidazol-1-yl)phenyl)urea, 1-(5-(6-(1H-pyrazol-4-yl)pyridin-2-yloxy)-2-fluorophenyl)-3-(3-(4-methyl-1H-imidazol-1-yl)-5-(trifluoromethyl)phenyl)urea, 1-(2-fluoro-5-(6-(1-methyl-1H-pyrazol-4-yl)pyridin-2-yloxy)phenyl)-3-(3-(pyrrolidin-1-yl)phenyl)urea, 1-(3-chloro-5-(pyrrolidin-1-yl)phenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-methyl-5-(pyrrolidin-1-yl)phenyl)urea, 1-(2-fluoro-5-(6-(1-methyl-1H-pyrazol-4-yl)pyridin-2-yloxy)phenyl)-3-(3-(pyrrolidin-1-yl)-5-(trifluoromethyl)phenyl)urea, 1-(2-fluoro-5-(6-(1-methyl-1H-pyrazol-4-yl)pyridin-2-yloxy)phenyl)-3-(3-(4-methyl-1H-imidazol-1-yl)phenyl)urea, 1-(3-chloro-5-(4-methyl-1H-imidazol-1-yl)phenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-methyl-5-(4-methyl-1H-imidazol-1-yl)phenyl)urea, 1-(2-fluoro-5-(6-(1-methyl-1H-pyrazol-4-yl)pyridin-2-yloxy)phenyl)-3-(3-(4-methyl-1H-imidazol-1-yl)-5-(trifluoromethyl)phenyl)urea, 1-(5-(2-(1H-pyrazol-4-yl)pyridin-4-yloxy)-2-fluorophenyl)-3-(3-(pyrrolidin-1-yl)phenyl)urea, 1-(5-(2-(1H-pyrazol-4-yl)pyridin-4-yloxy)-2-fluorophenyl)-3-(3-chloro-5-(pyrrolidin-1-yl)phenyl)urea, 1-(5-(2-(1H-pyrazol-4-yl)pyridin-4-yloxy)-2-fluorophenyl)-3-(3-methyl-5-(pyrrolidin-1-yl)phenyl)urea, 1-(5-(2-(1H-pyrazol-4-yl)pyridin-4-yloxy)-2-fluorophenyl)-3-(3-(pyrrolidin-1-yl)-5-(trifluoromethyl)phenyl)urea, 1-(5-(2-(1H-pyrazol-4-yl)pyridin-4-yloxy)-2-fluorophenyl)-3-(3-(4-methyl-1H-imidazol-1-yl)phenyl)urea, 1-(5-(2-(1H-pyrazol-4-yl)pyridin-4-yloxy)-2-fluorophenyl)-3-(3-chloro-5-(4-methyl-1H-imidazol-1-yl)phenyl)urea, 1-(5-(2-(1H-pyrazol-4-yl)pyridin-4-yloxy)-2-fluorophenyl)-3-(3-methyl-5-(4-methyl-1H-imidazol-1-yl)phenyl)urea, 1-(5-(2-(1H-pyrazol-4-yl)pyridin-4-yloxy)-2-fluorophenyl)-3-(3-(4-methyl-1H-imidazol-1-yl)-5-(trifluoromethyl)phenyl)urea, 1-(2-fluoro-5-(2-(1-methyl-1H-pyrazol-4-yl)pyridin-4-yloxy)phenyl)-3-(3-(pyrrolidin-1-yl)phenyl)urea, 1-(3-chloro-5-(pyrrolidin-1-yl)phenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-methyl-5-(pyrrolidin-1-yl)phenyl)urea, 1-(2-fluoro-5-(2-(1-methyl-1H-pyrazol-4-yl)pyridin-4-yloxy)phenyl)-3-(3-(pyrrolidin-1-yl)-5-(trifluoromethyl)phenyl)urea, 1-(2-fluoro-5-(2-(1-methyl-1H-pyrazol-4-yl)pyridin-4-yloxy)phenyl)-3-(3-(4-methyl-1H-imidazol-1-yl)phenyl)urea, 1-(3-chloro-5-(4-methyl-1H-imidazol-1-yl)phenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-methyl-5-(4-methyl-1H-imidazol-1-yl)phenyl)urea, 1-(2-fluoro-5-(2-(1-methyl-1H-pyrazol-4-yl)pyridin-4-yloxy)phenyl)-3-(3-(4-methyl-1H-imidazol-1-yl)-5-(trifluoromethyl)phenyl)urea, 1-(4-(2-(1H-pyrazol-4-yl)pyridin-4-yloxy)-2-fluorophenyl)-3-(3-(pyrrolidin-1-yl)phenyl)urea, 1-(4-(2-(1H-pyrazol-4-yl)pyridin-4-yloxy)-2-fluorophenyl)-3-(3-chloro-5-(pyrrolidin-1-yl)phenyl)urea, 1-(4-(2-(1H-pyrazol-4-yl)pyridin-4-yloxy)-2-fluorophenyl)-3-(3-methyl-5-(pyrrolidin-1-yl)phenyl)urea, 1-(4-(2-(1H-pyrazol-4-yl)pyridin-4-yloxy)-2-fluorophenyl)-3-(3-(pyrrolidin-1-yl)-5-(trifluoromethyl)phenyl)urea, 1-(4-(2-(1H-pyrazol-4-yl)pyridin-4-yloxy)-2-fluorophenyl)-3-(3-(4-methyl-1H-imidazol-1-yl)phenyl)urea, 1-(4-(2-(1H-pyrazol-4-yl)pyridin-4-yloxy)-2-fluorophenyl)-3-(3-chloro-5-(4-methyl-1H-imidazol-1-yl)phenyl)urea, 1-(4-(2-(1H-pyrazol-4-yl)pyridin-4-yloxy)-2-fluorophenyl)-3-(3-methyl-5-(4-methyl-1H-imidazol-1-yl)phenyl)urea, 1-(4-(2-(1H-pyrazol-4-yl)pyridin-4-yloxy)-2-fluorophenyl)-3-(3-(4-methyl-1H-imidazol-1-yl)-5-(trifluoromethyl)phenyl)urea, 1-(2-fluoro-4-(2-(1-methyl-1H-pyrazol-4-yl)pyridin-4-yloxy)phenyl)-3-(3-(pyrrolidin-1-yl)phenyl)urea, 1-(3-chloro-5-(pyrrolidin-1-yl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-(pyrrolidin-1-yl)phenyl)urea, 1-(2-fluoro-4-(2-(1-methyl-1H-pyrazol-4-yl)pyridin-4-yloxy)phenyl)-3-(3-(pyrrolidin-1-yl)-5-(trifluoromethyl)phenyl)urea, 1-(2-fluoro-4-(2-(1-methyl-1H-pyrazol-4-yl)pyridin-4-yloxy)phenyl)-3-(3-(4-methyl-1H-imidazol-1-yl)phenyl)urea, 1-(3-chloro-5-(4-methyl-1H-imidazol-1-yl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-(4-methyl-1H-imidazol-1-yl)phenyl)urea, 1-(2-fluoro-4-(2-(1-methyl-1H-pyrazol-4-yl)pyridin-4-yloxy)phenyl)-3-(3-(4-methyl-1H-imidazol-1-yl)-5-(trifluoromethyl)phenyl)urea, 1-(4-(2-(1H-pyrazol-4-yl)pyridin-4-yloxy)-3-fluorophenyl)-3-(3-(pyrrolidin-1-yl)phenyl)urea, 1-(4-(2-(1H-pyrazol-4-yl)pyridin-4-yloxy)-3-fluorophenyl)-3-(3-chloro-5-(pyrrolidin-1-yl)phenyl)urea, 1-(4-(2-(1H-pyrazol-4-yl)pyridin-4-yloxy)-3-fluorophenyl)-3-(3-methyl-5-(pyrrolidin-1-yl)phenyl)urea, 1-(4-(2-(1H-pyrazol-4-yl)pyridin-4-yloxy)-3-fluorophenyl)-3-(3-(pyrrolidin-1-yl)-5-(trifluoromethyl)phenyl)urea, 1-(4-(2-(1H-pyrazol-4-yl)pyridin-4-yloxy)-3-fluorophenyl)-3-(3-(4-methyl-1H-imidazol-1-yl)phenyl)urea, 1-(4-(2-(1H-pyrazol-4-yl)pyridin-4-yloxy)-3-fluorophenyl)-3-(3-chloro-5-(4-methyl-1H-imidazol-1-yl)phenyl)urea, 1-(4-(2-(1H-pyrazol-4-yl)pyridin-4-yloxy)-3-fluorophenyl)-3-(3-methyl-5-(4-methyl-1H-imidazol-1-yl)phenyl)urea, 1-(4-(2-(1H-pyrazol-4-yl)pyridin-4-yloxy)-3-fluorophenyl)-3-(3-(4-methyl-1H-imidazol-1-yl)-5-(trifluoromethyl)phenyl)urea, 1-(3-fluoro-4-(2-(1-methyl-1H-pyrazol-4-yl)pyridin-4-yloxy)phenyl)-3-(3-(pyrrolidin-1-yl)phenyl)urea, 1-(3-chloro-5-(pyrrolidin-1-yl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-(pyrrolidin-1-yl)phenyl)urea, 1-(3-fluoro-4-(2-(1-methyl-1H-pyrazol-4-yl)pyridin-4-yloxy)phenyl)-3-(3-(pyrrolidin-1-yl)-5-(trifluoromethyl)phenyl)urea, 1-(3-fluoro-4-(2-(1-methyl-1H-pyrazol-4-yl)pyridin-4-yloxy)phenyl)-3-(3-(4-methyl-1H-imidazol-1-yl)phenyl)urea, 1-(3-chloro-5-(4-methyl-1H-imidazol-1-yl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-(4-methyl-1H-imidazol-1-yl)phenyl)urea, 1-(3-fluoro-4-(2-(1-methyl-1H-pyrazol-4-yl)pyridin-4-yloxy)phenyl)-3-(3-(4-methyl-1H-imidazol-1-yl)-5-(trifluoromethyl)phenyl)urea, 1-(5-(2-(1H-pyrazol-4-yl)pyridin-4-yloxy)-2-fluorophenyl)-3-(3-(piperidin-1-yl)phenyl)urea, 1-(5-(2-(1H-pyrazol-4-yl)pyridin-4-yloxy)-2-fluorophenyl)-3-(3-methyl-5-(piperidin-1-yl)phenyl)urea, 1-(5-(2-(1H-pyrazol-4-yl)pyridin-4-yloxy)-2-fluorophenyl)-3-(3-chloro-5-(piperidin-1-yl)phenyl)urea, 1-(5-(2-(1H-pyrazol-4-yl)pyridin-4-yloxy)-2-fluorophenyl)-3-(3-(piperidin-1-yl)-5-(trifluoromethyl)phenyl)urea, 145-(2-(1H-pyrazol-4-yl)pyridin-4-yloxy)-2-fluorophenyl)-3-(3-(4-methylpiperazin-1-yl)phenyl)urea, 1-(5-(2-(1H-pyrazol-4-yl)pyridin-4-yloxy)-2-fluorophenyl)-3-(3-methyl-5-(4-methylpiperazin-1-yl)phenyl)urea, 1-(5-(2-(1H-pyrazol-4-yl)pyridin-4-yloxy)-2-fluorophenyl)-3-(3-chloro-5-(4-methylpiperazin-1-yl)phenyl)urea, 145-(2-(1H-pyrazol-4-yl)pyridin-4-yloxy)-2-fluorophenyl)-3-(3-(4-methylpiperazin-1-yl)-5-(trifluoromethyl)phenyl)urea, 1-(2-fluoro-5-(2-(1-methyl-1H-pyrazol-4-yl)pyridin-4-yloxy)phenyl)-3-(3-(piperidin-1-yl)phenyl)urea, 1-(2-fluoro-5-(2-(1-methyl-1H-pyrazol-4-yl)pyridin-4-yloxy)phenyl)-3-(3-methyl-5-(piperidin-1-yl)phenyl)urea, 1-(3-chloro-5-(piperidin-1-yl)phenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-(piperidin-1-yl)-5-(trifluoromethyl)phenyl)urea, 1-(5-(2-(1H-pyrazol-4-yl)pyridin-4-yloxy)-2-fluorophenyl)-3-(3-(4-methylpiperazin-1-yl)phenyl)urea, 1-(2-fluoro-5-(2-(1-methyl-1H-pyrazol-4-yl)pyridin-4-yloxy)phenyl)-3-(3-methyl-5-(4-methylpiperazin-1-yl)phenyl)urea, 1-(3-chloro-5-(4-methylpiperazin-1-yl)phenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-(4-methylpiperazin-1-yl)-5-(trifluoromethyl)phenyl)urea, 1-(5-(6-(1H-pyrazol-4-yl)pyridin-2-yloxy)-2-fluorophenyl)-3-(3-(piperidin-1-yl)phenyl)urea, 1-(5-(6-(1H-pyrazol-4-yl)pyridin-2-yloxy)-2-fluorophenyl)-3-(3-methyl-5-(piperidin-1-yl)phenyl)urea, 1-(5-(6-(1H-pyrazol-4-yl)pyridin-2-yloxy)-2-fluorophenyl)-3-(3-chloro-5-(piperidin-1-yl)phenyl)urea, 1-(5-(6-(1H-pyrazol-4-yl)pyridin-2-yloxy)-2-fluorophenyl)-3-(3-(piperidin-1-yl)-5-(trifluoromethyl)phenyl)urea, 1-(5-(6-(1H-pyrazol-4-yl)pyridin-2-yloxy)-2-fluorophenyl)-3-(3-(4-methylpiperazin-1-yl)phenyl)urea, 1-(5-(6-(1H-pyrazol-4-yl)pyridin-2-yloxy)-2-fluorophenyl)-3-(3-methyl-5-(4-methylpiperazin-1-yl)phenyl)urea, 1-(5-(6-(1H-pyrazol-4-yl)pyridin-2-yloxy)-2-fluorophenyl)-3-(3-chloro-5-(4-methylpiperazin-1-yl)phenyl)urea, 1-(5-(6-(1H-pyrazol-4-yl)pyridin-2-yloxy)-2-fluorophenyl)-3-(3-(4-methylpiperazin-1-yl)-5-(trifluoromethyl)phenyl)urea, 1-(2-fluoro-5-(6-(1-methyl-1H-pyrazol-4-yl)pyridin-2-yloxy)phenyl)-3-(3-(piperidin-1-yl)phenyl)urea, 1-(2-fluoro-5-(6-(1-methyl-1H-pyrazol-4-yl)pyridin-2-yloxy)phenyl)-3-(3-methyl-5-(piperidin-1-yl)phenyl)urea, 1-(3-chloro-5-(piperidin-1-yl)phenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-(piperidin-1-yl)-5-(trifluoromethyl)phenyl)urea, 1-(5-(6-(1H-pyrazol-4-yl)pyridin-2-yloxy)-2-fluorophenyl)-3-(3-(4-methylpiperazin-1-yl)phenyl)urea, 1-(2-fluoro-5-(6-(1-methyl-1H-pyrazol-4-yl)pyridin-2-yloxy)phenyl)-3-(3-methyl-5-(4-methylpiperazin-1-yl)phenyl)urea, 1-(3-chloro-5-(4-methylpiperazin-1-yl)phenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-(4-methylpiperazin-1-yl)-5-(trifluoromethyl)phenyl)urea, 1-(4-(2-(1H-pyrazol-4-yl)pyridin-4-yloxy)-2-fluorophenyl)-3-(3-(piperidin-1-yl)phenyl)urea, 1-(4-(2-(1H-pyrazol-4-yl)pyridin-4-yloxy)-2-fluorophenyl)-3-(3-chloro-5-(piperidin-1-yl)phenyl)urea, 1-(4-(2-(1H-pyrazol-4-yl)pyridin-4-yloxy)-2-fluorophenyl)-3-(3-methyl-5-(piperidin-1-yl)phenyl)urea, 1-(4-(2-(1H-pyrazol-4-yl)pyridin-4-yloxy)-2-fluorophenyl)-3-(3-(piperidin-1-yl)-5-(trifluoromethyl)phenyl)urea, 1-(4-(2-(1H-pyrazol-4-yl)pyridin-4-yloxy)-2-fluorophenyl)-3-(3-(4-methylpiperazin-1-yl)phenyl)urea, 1-(4-(2-(1H-pyrazol-4-yl)pyridin-4-yloxy)-2-fluorophenyl)-3-(3-chloro-5-(4-methylpiperazin-1-yl)phenyl)urea, 1-(4-(2-(1H-pyrazol-4-yl)pyridin-4-yloxy)-2-fluorophenyl)-3-(3-methyl-5-(4-methylpiperazin-1-yl)phenyl)urea, 1-(4-(2-(1H-pyrazol-4-yl)pyridin-4-yloxy)-2-fluorophenyl)-3-(3-(4-methylpiperazin-1-yl)-5-(trifluoromethyl)phenyl)urea, 1-(2-fluoro-4-(2-(1-methyl-1H-pyrazol-4-yl)pyridin-4-yloxy)phenyl)-3-(3-(piperidin-1-yl)phenyl)urea, 1-(3-chloro-5-(piperidin-1-yl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-(piperidin-1-yl)phenyl)urea, 1-(2-fluoro-4-(2-(1-methyl-1H-pyrazol-4-yl)pyridin-4-yloxy)phenyl)-3-(3-(piperidin-1-yl)-5-(trifluoromethyl)phenyl)urea, 1-(2-fluoro-4-(2-(1-methyl-1H-pyrazol-4-yl)pyridin-4-yloxy)phenyl)-3-(3-(4-methylpiperazin-1-yl)phenyl)urea, 1-(3-chloro-5-(4-methylpiperazin-1-yl)phenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-(4-methylpiperazin-1-yl)phenyl)urea, 1-(2-fluoro-4-(2-(1-methyl-1H-pyrazol-4-yl)pyridin-4-yloxy)phenyl)-3-(3-(4-methylpiperazin-1-yl)-5-(trifluoromethyl)phenyl)urea, 1-(4-(2-(1H-pyrazol-4-yl)pyridin-4-yloxy)-3-fluorophenyl)-3-(3-(piperidin-1-yl)phenyl)urea, 1-(4-(2-(1H-pyrazol-4-yl)pyridin-4-yloxy)-3-fluorophenyl)-3-(3-chloro-5-(piperidin-1-yl)phenyl)urea, 1-(4-(2-(1H-pyrazol-4-yl)pyridin-4-yloxy)-3-fluorophenyl)-3-(3-methyl-5-(piperidin-1-yl)phenyl)urea, 1-(4-(2-(1H-pyrazol-4-yl)pyridin-4-yloxy)-3-fluorophenyl)-3-(3-(piperidin-1-yl)-5-(trifluoromethyl)phenyl)urea, 1-(4-(2-(1H-pyrazol-4-yl)pyridin-4-yloxy)-3-fluorophenyl)-3-(3-(4-methylpiperazin-1-yl)phenyl)urea, 1-(4-(2-(1H-pyrazol-4-yl)pyridin-4-yloxy)-3-fluorophenyl)-3-(3-chloro-5-(4-methylpiperazin-1-yl)phenyl)urea, 1-(4-(2-(1H-pyrazol-4-yl)pyridin-4-yloxy)-3-fluorophenyl)-3-(3-methyl-5-(4-methylpiperazin-1-yl)phenyl)urea, 1-(4-(2-(1H-pyrazol-4-yl)pyridin-4-yloxy)-3-fluorophenyl)-3-(3-(4-methylpiperazin-1-yl)-5-(trifluoromethyl)phenyl)urea, 1-(3-fluoro-4-(2-(1-methyl-1H-pyrazol-4-yl)pyridin-4-yloxy)phenyl)-3-(3-(piperidin-1-yl)phenyl)urea, 1-(3-chloro-5-(piperidin-1-yl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-(piperidin-1-yl)phenyl)urea, 1-(3-fluoro-4-(2-(1-methyl-1H-pyrazol-4-yl)pyridin-4-yloxy)phenyl)-3-(3-(piperidin-1-yl)-5-(trifluoromethyl)phenyl)urea, 1-(3-fluoro-4-(2-(1-methyl-1H-pyrazol-4-yl)pyridin-4-yloxy)phenyl)-3-(3-(4-methylpiperazin-1-yl)phenyl)urea, 1-(3-chloro-5-(4-methylpiperazin-1-yl)phenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-(4-methylpiperazin-1-yl)phenyl)urea, 1-(3-fluoro-4-(2-(1-methyl-1H-pyrazol-4-yl)pyridin-4-yloxy)phenyl)-3-(3-(4-methylpiperazin-1-yl)-5-(trifluoromethyl)phenyl)urea, 1-(5-(6-(1H-pyrazol-4-yl)pyridin-2-yloxy)-2-fluorophenyl)-3-(3-morpholinophenyl)urea, 1-(5-(6-(1H-pyrazol-4-yl)pyridin-2-yloxy)-2-fluorophenyl)-3-(3-methyl-5-(piperidin-1-yl)phenyl)urea, 1-(5-(6-(1H-pyrazol-4-yl)pyridin-2-yloxy)-2-fluorophenyl)-3-(3-chloro-5-morpholinophenyl)urea, 1-(5-(6-(1H-pyrazol-4-yl)pyridin-2-yloxy)-2-fluorophenyl)-3-(3-morpholino-5-(trifluoromethyl)phenyl)urea, 1-(2-fluoro-5-(6-(1-methyl-1H-pyrazol-4-yl)pyridin-2-yloxy)phenyl)-3-(3-morpholinophenyl)urea, 1-(2-fluoro-5-(6-(1-methyl-1H-pyrazol-4-yl)pyridin-2-yloxy)phenyl)-3-(3-methyl-5-morp holinophenyl)urea, 1-(3-chloro-5-morpholinophenyl)-3-(2-fluoro-5-(6-(1-methyl-1H-pyrazol-4-yl)pyridin-2-yloxy)phenyl)urea, 1-(2-fluoro-5-(6-(1-methyl-1H-pyrazol-4-yl)pyridin-2-yloxy)phenyl)-3-(3-morpholino-5-(trifluoromethyl)phenyl)urea, 1-(5-(2-(1H-pyrazol-4-yl)pyridin-4-yloxy)-2-fluorophenyl)-3-(3-morpholinophenyl)urea, 145-(2-(1H-pyrazol-4-yl)pyridin-4-yloxy)-2-fluorophenyl)-3-(3-methyl-5-(piperidin-1-yl)phenyl)urea, 1-(5-(2-(1H-pyrazol-4-yl)pyridin-4-yloxy)-2-fluorophenyl)-3-(3-chloro-5-morpholinophenyl)urea, 1-(5-(2-(1H-pyrazol-4-yl)pyridin-4-yloxy)-2-fluorophenyl)-3-(3-morpholino-5-(trifluoromethyl)phenyl)urea, 1-(2-fluoro-5-(2-(1-methyl-1H-pyrazol-4-yl)pyridin-4-yloxy)phenyl)-3-(3-morpholinophenyl)urea, 1-(2-fluoro-5-(2-(1-methyl-1H-pyrazol-4-yl)pyridin-4-yloxy)phenyl)-3-(3-methyl-5-morpholinophenyl)urea, 1-(3-chloro-5-morpholinophenyl)-3-(2-fluoro-5-(2-(1-methyl-1H-pyrazol-4-yl)pyridin-4-yloxy)phenyl)urea, 1-(2-fluoro-5-(2-(1-methyl-1H-pyrazol-4-yl)pyridin-4-yloxy)phenyl)-3-(3-morpholino-5-(trifluoromethyl)phenyl)urea, 1-(4-(2-(1H-pyrazol-4-yl)pyridin-4-yloxy)-2-fluorophenyl)-3-(3-morpholinophenyl)urea, 1-(4-(2-(1H-pyrazol-4-yl)pyridin-4-yloxy)-2-fluorophenyl)-3-(3-chloro-5-morpholinophenyl)urea, 1-(4-(2-(1H-pyrazol-4-yl)pyridin-4-yloxy)-2-fluorophenyl)-3-(3-methyl-5-morpholinophenyl)urea, 1-(4-(2-(1H-pyrazol-4-yl)pyridin-4-yloxy)-2-fluorophenyl)-3-(3-morpholino-5-(trifluoromethyl)phenyl)urea, 1-(2-fluoro-4-(2-(1-methyl-1H-pyrazol-4-yl)pyridin-4-yloxy)phenyl)-3-(3-morpholinophenyl)urea, 1-(3-chloro-5-morpholinophenyl)-3-(2-fluoro-4-(2-(1-methyl-1H-pyrazol-4-yl)pyridin-4-yloxy)phenyl)urea, 1-(2-fluoro-4-(2-(1-methyl-1H-pyrazol-4-yl)pyridin-4-yloxy)phenyl)-3-(3-methyl-5-morp holinophenyl)urea, 1-(2-fluoro-4-(2-(1-methyl-1H-pyrazol-4-yl)pyridin-4-yloxy)phenyl)-3-(3-morpholino-5-(trifluoromethyl)phenyl)urea, 1-(4-(2-(1H-pyrazol-4-yl)pyridin-4-yloxy)-3-fluorophenyl)-3-(3-morpholinophenyl)urea, 1-(4-(2-(1H-pyrazol-4-yl)pyridin-4-yloxy)-3-fluorophenyl)-3-(3-chloro-5-morpholinophenyl)urea, 1-(4-(2-(1H-pyrazol-4-yl)pyridin-4-yloxy)-3-fluorophenyl)-3-(3-methyl-5-morpholinophenyl)urea, 1-(4-(2-(1H-pyrazol-4-yl)pyridin-4-yloxy)-3-fluorophenyl)-3-(3-morpholino-5-(trifluoromethyl)phenyl)urea, 1-(3-fluoro-4-(2-(1-methyl-1H-pyrazol-4-yl)pyridin-4-yloxy)phenyl)-3-(3-morpholinophenyl)urea, 1-(3-chloro-5-morpholinophenyl)-3-(3-fluoro-4-(2-(1-methyl-1H-pyrazol-4-yl)pyridin-4-yloxy)phenyl)urea, 1-(3-fluoro-4-(2-(1-methyl-1H-pyrazol-4-yl)pyridin-4-yloxy)phenyl)-3-(3-methyl-5-morpholinophenyl)urea, 1-(3-fluoro-4-(2-(1-methyl-1H-pyrazol-4-yl)pyridin-4-yloxy)phenyl)-3-(3-morpholino-5-(trifluoromethyl)phenyl)urea, 1-(2-fluoro-4-(2-(1-methyl-1H-pyrazol-4-yl)pyridin-4-yloxy)phenyl)-3-(6-(pyrrolidin-1-yl)benzo[d]thiazol-2-yl)urea, 1-(2-fluoro-4-(2-(1-methyl-1H-pyrazol-4-yl)pyridin-4-yloxy)phenyl)-3-(6-(4-methyl-1H-imidazol-1-yl)benzo[d]thiazol-2-yl)urea, 1-(2-fluoro-4-(2-(1-methyl-1H-pyrazol-4-yl)pyridin-4-yloxy)phenyl)-3-(6-(piperidin-1-yl)benzo[d]thiazol-2-yl)urea, 1-(2-fluoro-4-(2-(1-methyl-1H-pyrazol-4-yl)pyridin-4-yloxy)phenyl)-3-(6-morpholinobenzo[d]thiazol-2-yl)urea, 1-(2-fluoro-4-(2-(1-methyl-1H-pyrazol-4-yl)pyridin-4-yloxy)phenyl)-3-(6-(4-methylpiperazin-1-yl)benzo[d]thiazol-2-yl)urea, 1-(3-fluoro-4-(2-(1-methyl-1H-pyrazol-4-yl)pyridin-4-yloxy)phenyl)-3-(6-(pyrrolidin-1-yl)benzo[d]thiazol-2-yl)urea, 1-(3-fluoro-4-(2-(1-methyl-1H-pyrazol-4-yl)pyridin-4-yloxy)phenyl)-3-(6-(4-methyl-1H-imidazol-1-yl)benzo[d]thiazol-2-yl)urea, 1-(3-fluoro-4-(2-(1-methyl-1H-pyrazol-4-yl)pyridin-4-yloxy)phenyl)-3-(6-(piperidin-1-yl)benzo[d]thiazol-2-yl)urea, 1-(3-fluoro-4-(2-(1-methyl-1H-pyrazol-4-yl)pyridin-4-yloxy)phenyl)-3-(6-morpholinobenzo[d]thiazol-2-yl)urea, 1-(3-fluoro-4-(2-(1-methyl-1H-pyrazol-4-yl)pyridin-4-yloxy)phenyl)-3-(6-(4-methylpiperazin-1-yl)benzo[d]thiazol-2-yl)urea, 1-(2-fluoro-4-(2-(1-methyl-1H-pyrazol-4-yl)pyridin-4-yloxy)phenyl)-3-(2-oxo-6-(pyrrolidin-1-yl)indolin-3-yl)urea, 1-(2-fluoro-4-(2-(1-methyl-1H-pyrazol-4-yl)pyridin-4-yloxy)phenyl)-3-(6-(4-methyl-1H-imidazol-1-yl)-2-oxoindolin-3-yl)urea, 1-(2-fluoro-4-(2-(1-methyl-1H-pyrazol-4-yl)pyridin-4-yloxy)phenyl)-3-(2-oxo-6-(piperidin-1-yl)indolin-3-yl)urea, 1-(2-fluoro-4-(2-(1-methyl-1H-pyrazol-4-yl)pyridin-4-yloxy)phenyl)-3-(6-morpholino-2-oxoindolin-3-yl)urea, 1-(2-fluoro-4-(2-(1-methyl-1H-pyrazol-4-yl)pyridin-4-yloxy)phenyl)-3-(6-(4-methylpiperazin-1-yl)-2-oxoindolin-3-yl)urea, 1-(3-fluoro-4-(2-(1-methyl-1H-pyrazol-4-yl)pyridin-4-yloxy)phenyl)-3-(2-oxo-6-(pyrrolidin-1-yl)indolin-3-yl)urea, 1-(3-fluoro-4-(2-(1-methyl-1H-pyrazol-4-yl)pyridin-4-yloxy)phenyl)-3-(6-(4-methyl-1H-imidazol-1-yl)-2-oxoindolin-3-yl)urea, 1-(3-fluoro-4-(2-(1-methyl-1H-pyrazol-4-yl)pyridin-4-yloxy)phenyl)-3-(2-oxo-6-(piperidin-1-yl)indolin-3-yl)urea, 1-(3-fluoro-4-(2-(1-methyl-1H-pyrazol-4-yl)pyridin-4-yloxy)phenyl)-3-(6-morpholino-2-oxoindolin-3-yl)urea, 1-(3-fluoro-4-(2-(1-methyl-1H-pyrazol-4-yl)pyridin-4-yloxy)phenyl)-3-(6-(4-methylpiperazin-1-yl)-2-oxoindolin-3-yl)urea, 1-(2-fluoro-4-(2-(1-methyl-1H-pyrazol-4-yl)pyridin-4-yloxy)phenyl)-3-(2-(pyrrolidin-1-yl)quinolin-6-yl)urea, 1-(2-fluoro-4-(2-(1-methyl-1H-pyrazol-4-yl)pyridin-4-yloxy)phenyl)-3-(2-(4-methyl-1H-imidazol-1-yl)quinolin-6-yl)urea, 1-(2-fluoro-4-(2-(1-methyl-1H-pyrazol-4-yl)pyridin-4-yloxy)phenyl)-3-(2-(piperidin-1-yl)quinolin-6-yl)urea, 1-(2-fluoro-4-(2-(1-methyl-1H-pyrazol-4-yl)pyridin-4-yloxy)phenyl)-3-(2-morpholinoquinolin-6-yl)urea, 1-(2-fluoro-4-(2-(1-methyl-1H-pyrazol-4-yl)pyridin-4-yloxy)phenyl)-3-(2-(4-methylpiperazin-1-yl)quinolin-6-yl)urea, 1-(3-fluoro-4-(2-(1-methyl-1H-pyrazol-4-yl)pyridin-4-yloxy)phenyl)-3-(2-(pyrrolidin-1-yl)quinolin-6-yl)urea, 1-(3-fluoro-4-(2-(1-methyl-1H-pyrazol-4-yl)pyridin-4-yloxy)phenyl)-3-(2-(4-methyl-1H-imidazol-1-yl)quinolin-6-yl)urea, 1-(3-fluoro-4-(2-(1-methyl-1H-pyrazol-4-yl)pyridin-4-yloxy)phenyl)-3-(2-(piperidin-1-yl)quinolin-6-yl)urea, 1-(3-fluoro-4-(2-(1-methyl-1H-pyrazol-4-yl)pyridin-4-yloxy)phenyl)-3-(2-morpholinoquinolin-6-yl)urea, 1-(3-fluoro-4-(2-(1-methyl-1H-pyrazol-4-yl)pyridin-4-yloxy)phenyl)-3-(2-(4-methylpiperazin-1-yl)quinolin-6-yl)urea, 1-(2-fluoro-4-(2-(1-methyl-1H-pyrazol-4-yl)pyridin-4-yloxy)phenyl)-3-(3-(1-hydroxy-2-methylpropan-2-yl)-1-methyl-1H-pyrazol-5-yl)urea, 1-(2-fluoro-4-(2-(1-methyl-1H-pyrazol-4-yl)pyridin-4-yloxy)phenyl)-3-(3-(1-hydroxy-2-methylpropan-2-yl)isoxazol-5-yl)urea, 1-(2-fluoro-4-(2-(1-methyl-1H-pyrazol-4-yl)pyridin-4-yloxy)phenyl)-3-(5-(2-hydroxypropan-2-yl)pyridin-3-yl)urea, 1-(3-tert-butyl-1-methyl-1H-pyrazol-5-yl)-3-(2-fluoro-4-(2-(1-(2-hydroxyethyl)-1H-pyrazol-4-yl)pyridin-4-yloxy)phenyl)urea, 1-(3-tert-butyl-1-methyl-1H-pyrazol-5-yl)-3-(4-(2-(1-(2-(dimethylamino)ethyl)-1H-pyrazol-4-yl)pyridin-4-yloxy)-2-fluorophenyl)urea, 1-(3-tert-butyl-1-methyl-1H-pyrazol-5-yl)-3-(4-(2-(1-(cyanomethyl)-1H-pyrazol-4-yl)pyridin-4-yloxy)-2-fluorophenyl)urea, 1-(4-(2-(1-(2-amino-2-oxo ethyl)-1H-pyrazol-4-yl)pyridin-4-yloxy)-2,3-difluorophenyl)-3-(5-isopropylpyridin-3-yl)urea, 1-(4-(2-(1-(cyanomethyl)-1H-pyrazol-4-yl)pyridin-4-yloxy)-2,3-difluorophenyl)-3-(5-isopropylpyridin-3-yl)urea, 1-(2,3-difluoro-4-(2-(1-(2-morpholino ethyl)-1H-pyrazol-4-yl)pyridin-4-yloxy)phenyl)-3-(5-isopropylpyridin-3-yl)urea, 1-(2-fluoro-4-(2-(1-(2-morpholinoethyl)-1H-pyrazol-4-yl)pyridin-4-yloxy)phenyl)-3-(5-isopropylpyridin-3-yl)urea, 1-(2-fluoro-4-(2-(1-propyl-1H-pyrazol-4-yl)pyridin-4-yloxy)phenyl)-3-(5-isopropylpyridin-3-yl)urea, 1-(2,3-difluoro-4-(2-(1-(2-methoxyethyl)-1H-pyrazol-4-yl)pyridin-4-yloxy)phenyl)-3-(5-isopropylpyridin-3-yl)urea, 1-(2,3-difluoro-4-(2-(1-(2-hydroxyethyl)-1H-pyrazol-4-yl)pyridin-4-yloxy)phenyl)-3-(5-isopropylpyridin-3-yl)urea, 1-(4-(2-(1-(2-(dimethylamino)ethyl)-1H-pyrazol-4-yl)pyridin-4-yloxy)-2,3-difluorophenyl)-3-(5-isopropylpyridin-3-yl)urea, 1-(4-(2-(1-(3-(dimethylamino)propyl)-1H-pyrazol-4-yl)pyridin-4-yloxy)-2,3-difluorophenyl)-3-(5-isopropylpyridin-3-yl)urea, 1-(2,3-difluoro-4-(2-(1-(3-hydroxypropyl)-1H-pyrazol-4-yl)pyridin-4-yloxy)phenyl)-3-(5-isopropylpyridin-3-yl)urea, 1-(4-(2-(1-(2-(dimethylamino)ethyl)-1H-pyrazol-4-yl)pyridin-4-yloxy)-2-fluorophenyl)-3-(5-isopropylpyridin-3-yl)urea, 1-(2-fluoro-4-(2-(1-methyl-1H-pyrazol-4-yl)pyridin-4-yloxy)phenyl)-3-(4-(trifluoromethyl)pyridin-2-yl)urea, 1-(3-fluoro-4-(2-(1-(2-(4-methylpiperazin-1-yl)ethyl)-1H-pyrazol-4-yl)pyridin-4-ylo xy)phenyl)-3-(5-isopropylpyridin-3-yl)urea, 1-(2-fluoro-4-(2-(1-(3-hydroxypropyl)-1H-pyrazol-4-yl)pyridin-4-yloxy)phenyl)-3-(5-isopropylpyridin-3-yl)urea, 1-(2-fluoro-4-(2-(1-methyl-1H-pyrazol-4-yl)pyridin-4-yloxy)phenyl)-3-(4-(trifluoromethyl)pyridin-2-yl)urea, 1-(3-tert-butyl-1-methyl-1H-pyrazol-5-yl)-3-(4-(2-(1-(2-(dimethylamino)ethyl)-1H-pyrazol-4-yl)pyridin-4-yloxy)-2,3-difluorophenyl)urea, 1-(3-tert-butyl-1-methyl-1H-pyrazol-5-yl)-3-(2,3-difluoro-4-(2-(1-(2-methoxyethyl)-1H-pyrazol-4-yl)pyridin-4-yloxy)phenyl)urea, 1-(3-tert-butyl-1-methyl-1H-pyrazol-5-yl)-3-(2,3-difluoro-4-(2-(1-(2-hydroxyethyl)-1H-pyrazol-4-yl)pyridin-4-yloxy)phenyl)urea, 1-(3-tert-butyl-1-methyl-1H-pyrazol-5-yl)-3-(3-fluoro-4-(2-(1-(2-(4-methylpiperazin-1-yl)ethyl)-1H-pyrazol-4-yl)pyridin-4-yloxy)phenyl)urea, 1-(3-tert-butyl-1-methyl-1H-pyrazol-5-yl)-3-(4-(2-(1-(3-(dimethylamino)propyl)-1H-pyrazol-4-yl)pyridin-4-yloxy)-2,3-difluorophenyl)urea, 1-(3-tert-butyl-1-methyl-1H-pyrazol-5-yl)-3-(2,3-difluoro-4-(2-(1-(3-hydroxypropyl)-1H-pyrazol-4-yl)pyridin-4-yloxy)phenyl)urea, 1-(5-tert-butylpyridin-3-yl)-3-(2-fluoro-4-(2-(1-(3-hydroxypropyl)-1H-pyrazol-4-yl)pyridin-4-yloxy)phenyl)urea, and 1-(5-tert-butylpyridin-3-yl)-3-(2-fluoro-4-(2-(1-methyl-1H-pyrazol-4-yl)pyridin-4-yloxy)phenyl)urea.

### Section 4. Biological Data

c-ABL Kinase (Seq. ID no. 1) Assay

Activity of c-ABL kinase (Seq. ID no. 1) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A_{340 nm}) was continuously monitored spectrophometrically. The reaction mixture (100 μl) contained c-ABL kinase (1 nM. c-ABL from deCode Genetics), peptide substrate (EAIYAAPFAKKK, 0.2 mM), MgCl₂ (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 3.5% DMSO, pH 7.5. Test compounds were incubated with c-ABL (Seq. ID no. 1) and other reaction reagents at 30° C. for 2 h before ATP (500 μM) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

c-ABL kinase
(Seq. ID no. 1)
GTSMDPSSPNYDKWEMERTDITMKHKLGGGQYGEVYEGVWKKYSLTVAVKTLKEDTMEVE
EFLKEAAVMKEIKHPNLVQLLGVCTREPPFYIITEFMTYGNLLDYLRECNRQEVNAVVLL
YMATQISSAMEYLEKKNFIHRDLAARNCLVGENHLVKVADFGLSRLMTGDTYTAHAGAKF
PIKWTAPESLAYNKFSIKSDVWAFGVLLWEIATYGMSPYPGIDLSQVYELLEKDYRMERP
EGCPEKVYELMRACWQWNPSDRPSFAEIHQAFETMFQE

c-ABL Kinase (Seq. ID no. 2) Assay

Activity of T315I c-ABL kinase (Seq. ID no. 2) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A_{340 nm}) was continuously monitored spectrophometrically. The reaction mixture (100 μl) contained c-ABL kinase (4.4 nM. M315I c-ABL from deCode Genetics), peptide substrate (EAIYAAPFAKKK, 0.2 mM), MgCl₂ (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 1% DMSO, pH 7.5. Test compounds were incubated with T315I c-ABL (Seq. ID no. 2) and other reaction reagents at 30° C. for 1 h before ATP (500 μM) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC₅₀ values were calculated from a series of percent inhibition values determined at a range of inhibitor concentrations using software routines as implemented in the GraphPad Prism software package.

c-ABL T315I kinase
(Seq. ID no. 2)
GTSMDPSSPNYDKWEMERTDITMKHKLGGGQYGEVYEGVWKKYSLTVAVKTLKEDTMEVE
EFLKEAAVMKEIKHPNLVQLLGVCTREPPFYIIIEFMTYGNLLDYLRECNRQEVNAVVLL
YMATQISSAMEYLEKKNFIHRDLAARNCLVGENHLVKVADFGLSRLMTGDTYTAHAGAKF
PIKWTAPESLAYNKFSIKSDVWAFGVLLWEIATYGMSPYPGIDLSQVYELLEKDYRMERP
EGCPEKVYELMRACWQWNPSDRPSFAEIHQAFETMFQE
BCR-ABL p210-e14a2
(Seq. ID no. 3)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDADGSFGTPPGYGCAADRAEEQRRHQDGLPYIDDSPSSSPHLSSKGRGSRDALVSG
ALKSTKASELDLEKGLEMRKWVLSGILASEETYLSHLEALLLPMKPLKAAATTSQPVLTS
QQIETIFFKVPELYEIHKESYDGLFPRVQQWSHQQRVGDLFQKLASQLGVYRAFVDNYGV
AMEMAEKCCQANAQFAEISENLRARSNKDAKDPTTKNSLETLLYKPVDRVTRSTLVLHDL
LKHTPASHPDHPLLQDALRISQNFLSSINEEITPRRQSMTVKKGEHRQLLKDSFMVELVE
GARKLRHVFLFTDLLLCTKLKKQSGGKTQQYDCKWYIPLTDLSFQMVDELEAVPNIPLVP
DEELDALKIKISQIKSDIQREKRANKGSKATERLKKKLSEQESLLLLMSPSMAFRVHSRN
GKSYTFLISSDYERAEWRENIREQQKKCFRSFSLTSVELQMLTNSCVKLQTVHSIPLTIN
KEDDESPGLYGFLNVIVHSATGFKQSSKALQRPVASDFEPQGLSEAARWNSKENLLAGPS
ENDPNLFVALYDFVASGDNTLSITKGEKLRVLGYNHNGEWCEAQTKNGQGWVPSNYITPV
NSLEKHSWYHGPVSRNAAEYPLSSGINGSFLVRESESSPSQRSISLRYEGRVYHYRINTA
SDGKLYVSSESRFNTLAELVHHHSTVADGLITTLHYPAPKRNKPTVYGVSPNYDKWEMER
TDITMKHKLGGGQYGEVYEGVWKKYSLTVAVKTLKEDTMEVEEFLKEAAVMKEIKHPNLV
QLLGVCTREPPFYIITEFMTYGNLLDYLRECNRQEVNAVVLLYMATQISSAMEYLEKKNF
IHRDLAARNCLVGENHLVKVADFGLSRLMTGDTYTAHAGAKFPIKWTAPESLAYNKFSIK
SDVWAFGVLLWEIATYGMSPYPGIDRSQVYELLEKDYRMKRPEGCPEKVYELMRACWQWN
PSDRPSFAEIHQAFETMFQESSISDEVEKELGKQGVRGAVTTLLQAPELPTKTRTSRRAA
EHRDTTDVPEMPHSKGQGESDPLDHEPAVSPLLPRKERGPPEGGLNEDERLLPKDKKTNL
FSALIKKKKKTAPTPPKRSSSFREMDGQPERRGAGEEEGRDISNGALAFTPLDTADPAKS
PKPSNGAGVPNGALRESGGSGFRSPHLWKKSSTLTSSRLATGEEEGGGSSSKRFLRSCSV
SCVPHGAKDTEWRSVTLPRDLQSTGRQFDSSTFGGHKSEKPALPRKRAGENRSDQVTRGT
VTPPPRLVKKNEEAADEVFKDIMESSPGSSPPNLTPKPLRRQVTVAPASGLPHKEEAWKG
SALGTPAAAEPVTPTSKAGSGAPRGTSKGPAEESRVRRHKHSSESPGRDKGKLSKLKPAP
PPPPAASAGKAGGKPSQRPGQEAAGEAVLGAKTKATSLVDAVNSDAAKPSQPAEGLKKPV
LPATPKPHPAKPSGTPISPAPVPLSTLPSASSALAGDQPSSTAFIPLISTRVSLRKTRQP
PERASGAITKGVVLDSTEALCLAISGNSEQMASHSAVLEAGKNLYTFCVSYVDSIQQMRN
KFAFREAINKLENNLRELQICPASAGSGPAATQDFSKLLSSVKEISDIVQR
BCR-ABL p210-e13a2
(Seq. ID no. 4)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDADGSFGTPPGYGCAADRAEEQRRHQDGLPYIDDSPSSSPHLSSKGRGSRDALVSG
ALKSTKASELDLEKGLEMRKWVLSGILASEETYLSHLEALLLPMKPLKAAATTSQPVLTS
QQIETIFFKVPELYEIHKESYDGLFPRVQQWSHQQRVGDLFQKLASQLGVYRAFVDNYGV
AMEMAEKCCQANAQFAEISENLRARSNKDAKDPTTKNSLETLLYKPVDRVTRSTLVLHDL
LKHTPASHPDHPLLQDALRISQNFLSSINEEITPRRQSMTVKKGEHRQLLKDSFMVELVE
GARKLRHVFLFTDLLLCTKLKKQSGGKTQQYDCKWYIPLTDLSFQMVDELEAVPNIPLVP
DEELDALKIKISQIKSDIQREKRANKGSKATERLKKKLSEQESLLLLMSPSMAFRVHSRN
GKSYTFLISSDYERAEWRENIREQQKKCFRSFSLTSVELQMLTNSCVKLQTVHSIPLTIN
KEEALQRPVASDFEPQGLSEAARWNSKENLLAGPSENDPNLFVALYDFVASGDNTLSITK
GEKLRVLGYNHNGEWCEAQTKNGQGWVPSNYITPVNSLEKHSWYHGPVSRNAAEYPLSSG
INGSFLVRESESSPSQRSISLRYEGRVYHYRINTASDGKLYVSSESRFNTLAELVHHHST
VADGLITTLHYPAPKRNKPTVYGVSPNYDKWEMERTDITMKHKLGGGQYGEVYEGVWKKY
SLTVAVKTLKEDTMEVEEFLKEAAVMKEIKHPNLVQLLGVCTREPPFYIITEFMTYGNLL
DYLRECNRQEVNAVVLLYMATQISSAMEYLEKKNFIHRDLAARNCLVGENHLVKVADFGL
SRLMTGDTYTAHAGAKFPIKWTAPESLAYNKFSIKSDVWAFGVLLWEIATYGMSPYPGID
RSQVYELLEKDYRMKRPEGCPEKVYELMRACWQWNPSDRPSFAEIHQAFETMFQESSISD
EVEKELGKQGVRGAVTTLLQAPELPTKTRTSRRAAEHRDTTDVPEMPHSKGQGESDPLDH
EPAVSPLLPRKERGPPEGGLNEDERLLPKDKKTNLFSALIKKKKKTAPTPPKRSSSFREM
DGQPERRGAGEEEGRDISNGALAFTPLDTADPAKSPKPSNGAGVPNGALRESGGSGFRSP
HLWKKSSTLTSSRLATGEEEGGGSSSKRFLRSCSVSCVPHGAKDTEWRSVTLPRDLQSTG
RQFDSSTFGGHKSEKPALPRKRAGENRSDQVTRGTVTPPPRLVKKNEEAADEVFKDIMES
SPGSSPPNLTPKPLRRQVTVAPASGLPHKEEAWKGSALGTPAAAEPVTPTSKAGSGAPRG
TSKGPAEESRVRRHKHSSESPGRDKGKLSKLKPAPPPPPAASAGKAGGKPSQRPGQEAAG
EAVLGAKTKATSLVDAVNSDAAKPSQPAEGLKKPVLPATPKPHPAKPSGTPISPAPVPLS
TLPSASSALAGDQPSSTAFIPLISTRVSLRKTRQPPERASGAITKGVVLDSTEALCLAIS
GNSEQMASHSAVLEAGKNLYTFCVSYVDSIQQMRNKFAFREAINKLENNLRELQICPASA
GSGPAATQDFSKLLSSVKEISDIVQR
BCR-ABL p190-e1a2
(Seq. ID no. 5)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDAEALQRPVASDFEPQGLSEAARWNSKENLLAGPSENDPNLFVALYDFVASGDNTL
SITKGEKLRVLGYNHNGEWCEAQTKNGQGWVPSNYITPVNSLEKHSWYHGPVSRNAAEYP
LSSGINGSFLVRESESSPSQRSISLRYEGRVYHYRINTASDGKLYVSSESRFNTLAELVH
HHSTVADGLITTLHYPAPKRNKPTVYGVSPNYDKWEMERTDITMKHKLGGGQYGEVYEGV
WKKYSLTVAVKTLKEDTMEVEEFLKEAAVMKEIKHPNLVQLLGVCTREPPFYIITEFMTY
GNLLDYLRECNRQEVNAVVLLYMATQISSAMEYLEKKNFIHRDLAARNCLVGENHLVKVA
DFGLSRLMTGDTYTAHAGAKFPIKWTAPESLAYNKFSIKSDVWAFGVLLWEIATYGMSPY
PGIDRSQVYELLEKDYRMKRPEGCPEKVYELMRACWQWNPSDRPSFAEIHQAFETMFQES
SISDEVEKELGKQGVRGAVTTLLQAPELPTKTRTSRRAAEHRDTTDVPEMPHSKGQGESD
PLDHEPAVSPLLPRKERGPPEGGLNEDERLLPKDKKTNLFSALIKKKKKTAPTPPKRSSS
FREMDGQPERRGAGEEEGRDISNGALAFTPLDTADPAKSPKPSNGAGVPNGALRESGGSG
FRSPHLWKKSSTLTSSRLATGEEEGGGSSSKRFLRSCSVSCVPHGAKDTEWRSVTLPRDL
QSTGRQFDSSTFGGHKSEKPALPRKRAGENRSDQVTRGTVTPPPRLVKKNEEAADEVFKD
IMESSPGSSPPNLTPKPLRRQVTVAPASGLPHKEEAWKGSALGTPAAAEPVTPTSKAGSG
APRGTSKGPAEESRVRRHKHSSESPGRDKGKLSKLKPAPPPPPAASAGKAGGKPSQRPGQ
EAAGEAVLGAKTKATSLVDAVNSDAAKPSQPAEGLKKPVLPATPKPHPAKPSGTPISPAP
VPLSTLPSASSALAGDQPSSTAFIPLISTRVSLRKTRQPPERASGAITKGVVLDSTEALC
LAISGNSEQMASHSAVLEAGKNLYTFCVSYVDSIQQMRNKFAFREAINKLENNLRELQIC
PASAGSGPAATQDFSKLLSSVKEISDIVQR
BCR-ABL p210-e14a2 T315I
(Seq. ID no. 6)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDADGSFGTPPGYGCAADRAEEQRRHQDGLPYIDDSPSSSPHLSSKGRGSRDALVSG
ALKSTKASELDLEKGLEMRKWVLSGILASEETYLSHLEALLLPMKPLKAAATTSQPVLTS
QQIETIFFKVPELYEIHKESYDGLFPRVQQWSHQQRVGDLFQKLASQLGVYRAFVDNYGV
AMEMAEKCCQANAQFAEISENLRARSNKDAKDPTTKNSLETLLYKPVDRVTRSTLVLHDL
LKHTPASHPDHPLLQDALRISQNFLSSINEEITPRRQSMTVKKGEHRQLLKDSFMVELVE
GARKLRHVFLFTDLLLCTKLKKQSGGKTQQYDCKWYIPLTDLSFQMVDELEAVPNIPLVP
DEELDALKIKISQIKSDIQREKRANKGSKATERLKKKLSEQESLLLLMSPSMAFRVHSRN
GKSYTFLISSDYERAEWRENIREQQKKCFRSFSLTSVELQMLTNSCVKLQTVHSIPLTIN
KEDDESPGLYGFLNVIVHSATGFKQSSKALQRPVASDFEPQGLSEAARWNSKENLLAGPS
ENDPNLFVALYDFVASGDNTLSITKGEKLRVLGYNHNGEWCEAQTKNGQGWVPSNYITPV
NSLEKHSWYHGPVSRNAAEYPLSSGINGSFLVRESESSPSQRSISLRYEGRVYHYRINTA
SDGKLYVSSESRFNTLAELVHHHSTVADGLITTLHYPAPKRNKPTVYGVSPNYDKWEMER
TDITMKHKLGGGQYGEVYEGVWKKYSLTVAVKTLKEDTMEVEEFLKEAAVMKEIKHPNLV
QLLGVCTREPPFYIIIEFMTYGNLLDYLRECNRQEVNAVVLLYMATQISSAMEYLEKKNF
IHRDLAARNCLVGENHLVKVADFGLSRLMTGDTYTAHAGAKFPIKWTAPESLAYNKFSIK
SDVWAFGVLLWEIATYGMSPYPGIDRSQVYELLEKDYRMKRPEGCPEKVYELMRACWQWN
PSDRPSFAEIHQAFETMFQESSISDEVEKELGKQGVRGAVTTLLQAPELPTKTRTSRRAA
EHRDTTDVPEMPHSKGQGESDPLDHEPAVSPLLPRKERGPPEGGLNEDERLLPKDKKTNL
FSALIKKKKKTAPTPPKRSSSFREMDGQPERRGAGEEEGRDISNGALAFTPLDTADPAKS
PKPSNGAGVPNGALRESGGSGFRSPHLWKKSSTLTSSRLATGEEEGGGSSSKRFLRSCSV
SCVPHGAKDTEWRSVTLPRDLQSTGRQFDSSTFGGHKSEKPALPRKRAGENRSDQVTRGT
VTPPPRLVKKNEEAADEVFKDIMESSPGSSPPNLTPKPLRRQVTVAPASGLPHKEEAWKG
SALGTPAAAEPVTPTSKAGSGAPRGTSKGPAEESRVRRHKHSSESPGRDKGKLSKLKPAP
PPPPAASAGKAGGKPSQRPGQEAAGEAVLGAKTKATSLVDAVNSDAAKPSQPAEGLKKPV
LPATPKPHPAKPSGTPISPAPVPLSTLPSASSALAGDQPSSTAFIPLISTRVSLRKTRQP
PERASGAITKGVVLDSTEALCLAISGNSEQMASHSAVLEAGKNLYTFCVSYVDSIQQMRN
KFAFREAINKLENNLRELQICPASAGSGPAATQDFSKLLSSVKEISDIVQR
BCR-ABL p210-e13a2 T315I
(Seq. ID no. 7)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDADGSFGTPPGYGCAADRAEEQRRHQDGLPYIDDSPSSSPHLSSKGRGSRDALVSG
ALKSTKASELDLEKGLEMRKWVLSGILASEETYLSHLEALLLPMKPLKAAATTSQPVLTS
QQIETIFFKVPELYEIHKESYDGLFPRVQQWSHQQRVGDLFQKLASQLGVYRAFVDNYGV
AMEMAEKCCQANAQFAEISENLRARSNKDAKDPTTKNSLETLLYKPVDRVTRSTLVLHDL
LKHTPASHPDHPLLQDALRISQNFLSSINEEITPRRQSMTVKKGEHRQLLKDSFMVELVE
GARKLRHVFLFTDLLLCTKLKKQSGGKTQQYDCKWYIPLTDLSFQMVDELEAVPNIPLVP
DEELDALKIKISQIKSDIQREKRANKGSKATERLKKKLSEQESLLLLMSPSMAFRVHSRN
GKSYTFLISSDYERAEWRENIREQQKKCFRSFSLTSVELQMLTNSCVKLQTVHSIPLTIN
KEEALQRPVASDFEPQGLSEAARWNSKENLLAGPSENDPNLFVALYDFVASGDNTLSITK
GEKLRVLGYNHNGEWCEAQTKNGQGWVPSNYITPVNSLEKHSWYHGPVSRNAAEYPLSSG
INGSFLVRESESSPSQRSISLRYEGRVYHYRINTASDGKLYVSSESRFNTLAELVHHHST
VADGLITTLHYPAPKRNKPTVYGVSPNYDKWEMERTDITMKHKLGGGQYGEVYEGVWKKY
SLTVAVKTLKEDTMEVEEFLKEAAVMKEIKHPNLVQLLGVCTREPPFYIIIEFMTYGNLL
DYLRECNRQEVNAVVLLYMATQISSAMEYLEKKNFIHRDLAARNCLVGENHLVKVADFGL
SRLMTGDTYTAHAGAKFPIKWTAPESLAYNKFSIKSDVWAFGVLLWEIATYGMSPYPGID
RSQVYELLEKDYRMKRPEGCPEKVYELMRACWQWNPSDRPSFAEIHQAFETMFQESSISD
EVEKELGKQGVRGAVTTLLQAPELPTKTRTSRRAAEHRDTTDVPEMPHSKGQGESDPLDH
EPAVSPLLPRKERGPPEGGLNEDERLLPKDKKTNLFSALIKKKKKTAPTPPKRSSSFREM
DGQPERRGAGEEEGRDISNGALAFTPLDTADPAKSPKPSNGAGVPNGALRESGGSGFRSP
HLWKKSSTLTSSRLATGEEEGGGSSSKRFLRSCSVSCVPHGAKDTEWRSVTLPRDLQSTG
RQFDSSTFGGHKSEKPALPRKRAGENRSDQVTRGTVTPPPRLVKKNEEAADEVFKDIMES
SPGSSPPNLTPKPLRRQVTVAPASGLPHKEEAWKGSALGTPAAAEPVTPTSKAGSGAPRG
TSKGPAEESRVRRHKHSSESPGRDKGKLSKLKPAPPPPPAASAGKAGGKPSQRPGQEAAG
EAVLGAKTKATSLVDAVNSDAAKPSQPAEGLKKPVLPATPKPHPAKPSGTPISPAPVPLS
TLPSASSALAGDQPSSTAFIPLISTRVSLRKTRQPPERASGAITKGVVLDSTEALCLAIS
GNSEQMASHSAVLEAGKNLYTFCVSYVDSIQQMRNKFAFREAINKLENNLRELQICPASA
GSGPAATQDFSKLLSSVKEISDIVQR
BCR-ABL p190-e1a2
(Seq. ID no. 8)
MVDPVGFAEAWKAQFPDSEPPRMELRSVGDIEQELERCKASIRRLEQEVNQERFRMIYLQ
TLLAKEKKSYDRQRWGFRRAAQAPDGASEPRASASRPQPAPADGADPPPAEEPEARPDGE
GSPGKARPGTARRPGAAASGERDDRGPPASVAALRSNFERIRKGHGQPGADAEKPFYVNV
EFHHERGLVKVNDKEVSDRISSLGSQAMQMERKKSQHGAGSSVGDASRPPYRGRSSESSC
GVDGDYEDAELNPRFLKDNLIDANGGSRPPWPPLEYQPYQSIYVGGIMEGEGKGPLLRSQ
STSEQEKRLTWPRRSYSPRSFEDCGGGYTPDCSSNENLTSSEEDFSSGQSSRVSPSPTTY
RMFRDKSRSPSQNSQQSFDSSSPPTPQCHKRHRHCPVVVSEATIVGVRKTGQIWPNDDEG
AFHGDAEALQRPVASDFEPQGLSEAARWNSKENLLAGPSENDPNLFVALYDFVASGDNTL
SITKGEKLRVLGYNHNGEWCEAQTKNGQGWVPSNYITPVNSLEKHSWYHGPVSRNAAEYP
LSSGINGSFLVRESESSPSQRSISLRYEGRVYHYRINTASDGKLYVSSESRFNTLAELVH
HHSTVADGLITTLHYPAPKRNKPTVYGVSPNYDKWEMERTDITMKHKLGGGQYGEVYEGV
WKKYSLTVAVKTLKEDTMEVEEFLKEAAVMKEIKHPNLVQLLGVCTREPPFYIIIEFMTY
GNLLDYLRECNRQEVNAVVLLYMATQISSAMEYLEKKNFIHRDLAARNCLVGENHLVKVA
DFGLSRLMTGDTYTAHAGAKFPIKWTAPESLAYNKFSIKSDVWAFGVLLWEIATYGMSPY
PGIDRSQVYELLEKDYRMKRPEGCPEKVYELMRACWQWNPSDRPSFAEIHQAFETMFQES
SISDEVEKELGKQGVRGAVTTLLQAPELPTKTRTSRRAAEHRDTTDVPEMPHSKGQGESD
PLDHEPAVSPLLPRKERGPPEGGLNEDERLLPKDKKTNLFSALIKKKKKTAPTPPKRSSS
FREMDGQPERRGAGEEEGRDISNGALAFTPLDTADPAKSPKPSNGAGVPNGALRESGGSG
FRSPHLWKKSSTLTSSRLATGEEEGGGSSSKRFLRSCSVSCVPHGAKDTEWRSVTLPRDL
QSTGRQFDSSTFGGHKSEKPALPRKRAGENRSDQVTRGTVTPPPRLVKKNEEAADEVFKD
IMESSPGSSPPNLTPKPLRRQVTVAPASGLPHKEEAWKGSALGTPAAAEPVTPTSKAGSG
APRGTSKGPAEESRVRRHKHSSESPGRDKGKLSKLKPAPPPPPAASAGKAGGKPSQRPGQ
EAAGEAVLGAKTKATSLVDAVNSDAAKPSQPAEGLKKPVLPATPKPHPAKPSGTPISPAP
VPLSTLPSASSALAGDQPSSTAFIPLISTRVSLRKTRQPPERASGAITKGVVLDSTEALC
LAISGNSEQMASHSAVLEAGKNLYTFCVSYVDSIQQMRNKFAFREAINKLENNLRELQIC
PASAGSGPAATQDFSKLLSSVKEISDIVQR

c-KIT Kinase (Seq. ID no. 9) Assay

Activity of c-KIT kinase (Seq. ID no. 9) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A340 nm) was continuously monitored spectrophometrically. The reaction mixture (100 μl) contained c-KIT (cKIT residues T544-V976, from ProQinase, 5.4 nM), polyE4Y (1 mg/ml), MgCl2 (10 mM), pyruvate kinase (4 units), lactate dehydrogenase (0.7 units), phosphoenol pyruvate (1 mM), and NADH (0.28 mM) in 90 mM Tris buffer containing 0.2% octyl-glucoside and 1% DMSO, pH 7.5. Test compounds were incubated with C-MET (Seq. ID no. 9) and other reaction reagents at 22° C. for <2 min before ATP (200 μM) was added to start the reaction. The absorption at 340 nm was monitored continuously for 0.5 hours at 30° C. on Polarstar Optima plate reader (BMG). The reaction rate was calculated using the 0 to 0.5 h time frame. Percent inhibition was obtained by comparison of reaction rate with that of a control (i.e. with no test compound). IC50 values were calculated from a series of percent inhibition values determined at a range of inhibitor concentrations using software routines as implemented in the GraphPad Prism software package.

c-KIT with N-terminal GST fusion
(Seq ID no. 9)
LGYWKIKGLVQPTRLLLEYLEEKYEEHLYERDEGDKWRNKKFELGLEFPNLPYYIDGDVKL
TQSMAIIRYIADKHNMLGGCPKERAEISMLEGAVDIRYGVSRIAYSKDFETLKVDFLSKLP
EMLKMFEDRLCHKTYLNGDHVTHPDFMLYDALDVVLYMDPMCLDAFPKLVCFKKRIEAIPQ
IDKYLKSSKYIWPLQGWQATFGGGDHPPKSDLVPRHNQTSLYKKAGSAAAVLEENLYFQGT
YKYLQKPMYEVQWKVVEEINGNNYVYIDPTQLPYDHKWEFPRNRLSFGKTLGAGAFGKVVE
ATAYGLIKSDAAMTVAVKMLKPSAHLTEREALMSELKVLSYLGNHMNIVNLLGACTIGGPT
LVITEYCCYGDLLNFLRRKRDSFICSKQEDHAEAALYKNLLHSKESSCSDSTNEYMDMKPG
VSYVVPTKADKRRSVRIGSYIERDVTPAIMEDDELALDLEDLLSFSYQVAKGMAFLASKNC
IHRDLAARNILLTHGRITKICDFGLARDIKNDSNYVVKGNARLPVKWMAPESIFNCVYTFE
SDVWSYGIFLWELFSLGSSPYPGMPVDSKFYKMIKEGFRMLSPEHAPAEMYDIMKTCWDAD
PLKRPTFKQIVQLIEKQISESTNHIYSNLANCSPNRQKPVVDHSVRINSVGSTASSSQPL
LVHDDV

c-MET Kinase (Seq. ID no. 10) Assay

Activity of c-MET kinase (Seq. ID no. 10) was determined by following the production of ADP from the kinase reaction through coupling with the pyruvate kinase/lactate dehydrogenase system (e.g., Schindler, et al. Science (2000) 289, 1938-1942). In this assay, the oxidation of NADH (thus the decrease at A340 nm) was continuously monitored spectrophometrically. The reaction mixture (100 μl) contained c-MET (c-MET residues: 956-1390, from Invitrogen, catalogue #PV3143, 6 nM), polyE4Y (1 mg/ml), MgC12 (10 mM), pyruvate kinase (4 units), lactate dehydrogenase (0.7 units), phosphoenol pyruvate (1 mM), and NADH (0.28 mM) in 90 mM Tris buffer containing 0.25 mM DTT, 0.2% octyl-glucoside and 1% DMSO, pH 7.5. Test compounds were incubated with C-Met (Seq. ID no. 10) and other reaction reagents at 22° C. for 0.5 h before ATP (100 μM) was added to start the reaction. The absorption at 340 nm was monitored continuously for 2 hours at 30° C. on Polarstar Optima plate reader (BMG). The reaction rate was calculated using the 1.0 to 2.0 h time frame. Percent inhibition was obtained by comparison of reaction rate with that of a control (i.e. with no test compound). IC50 values were calculated from a series of percent inhibition values determined at a range of inhibitor concentrations using software routines as implemented in the GraphPad Prism software package.

c-MET Kinase
(Seq ID no. 10)
MSYYHHHHHHDYDIPTTENLYFQGAMLVPRGSPWIPFTMKKRKQIKDLGSELVRYDARVHT
PHLDRLVSARSVSPTTEMVSNESVDYRATFPEDQFPNSSQNGSCRQVQYPLTDMSPILTSG
DSDISSPLLQNTVHIDLSALNPELVQAVQHVVIGPSSLIVHFNEVIGRGHFGCVYHGTLLD
NDGKKIHCAVKSLNRITDIGEVSQFLTEGIIMKDFSHPNVLSLLGICLRSEGSPLVVLPYM
KHGDLRNFIRNETHNPTVKDLIGFGLQVAKGMKYLASKKFVHRDLAARNCMLDEKFTVKVA
DFGLARDMYDKEYYSVHNKTGAKLPVKWMALESLQTQKFTTKSDVWSFGVLLWELMTRGAP
PYPDVNTFDITVYLLQGRRLLQPEYCPDPLYEVMLKCWHPKAEMRPSFSELVSRISAIFST
FIGEHYVHVNATYVNVKCVAPYPSLLSSEDNADDEVDTRPASFWETS

TABLE 1
Biological Data Summary.
Biochemical IC₅₀ values of compounds of Formula I.

| | ABL | ABL T315I | c-KIT | c-MET |
|---|---|---|---|---|
| | Enzyme | Enzyme | Enzyme | Enzyme |
| Example | Assay | Assay | Assay | Assay |
| 1 | +++ | +++ | +++ | ++ |
| 2 | +++ | +++ | +++ | ++ |
| 3 | +++ | +++ | +++ | ++ |
| 4 | +++ | +++ | n/a | ++ |
| 5 | +++ | +++ | +++ | + |
| 6 | +++ | +++ | +++ | + |
| 7 | +++ | +++ | n/a | + |
| 8 | +++ | +++ | +++ | ++ |
| 9 | +++ | ‡ | +++ | + |
| 10 | +++ | + | +++ | + |
| 11 | +++ | +++ | +++ | + |
| 12 | +++ | +++ | +++ | + |
| 13 | +++ | + | n/a | ++ |
| 14 | +++ | +++ | +++ | ++ |
| 15 | +++ | +++ | +++ | + |
| 16 | +++ | ++ | n/a | + |
| 17 | +++ | +++ | n/a | ++ |
| 18 | +++ | n/a | n/a | + |
| 19 | ++ | ++ | n/a | + |
| 20 | +++ | +++ | n/a | + |
| 21 | +++ | +++ | +++ | ++ |
| 22 | +++ | +++ | +++ | ++ |
| 23 | +++ | n/a | n/a | n/a |
| 24 | +++ | n/a | +++ | + |
| 25 | +++ | +++ | +++ | +++ |
| 26 | +++ | n/a | n/a | n/a |
| 27 | +++ | +++ | +++ | ++ |
| 28 | +++ | +++ | +++ | + |
| 29 | +++ | n/a | n/a | n/a |
| 30 | +++ | +++ | n/a | + |
| 31 | +++ | +++ | n/a | n/a |
| 32 | +++ | +++ | n/a | n/a |
| 33 | ++ | ++ | n/a | n/a |
| 34 | +++ | +++ | n/a | n/a |
| 35 | ++ | + | n/a | n/a |
| 36 | ++ | + | n/a | n/a |
| 37 | +++ | +++ | n/a | n/a |
| 38 | +++ | ++ | n/a | n/a |
| 39 | +++ | +++ | n/a | n/a |
| 40 | +++ | ++ | n/a | ‡ |
| 41 | ++ | ‡ | ‡ | ‡ |
| 42 | +++ | +++ | +++ | ++ |

+++ = <0.1 μM;
++ = <1.0 μM;
+ = <10 μM;
‡ <100 μM;
n/a = not available

The biochemical IC₅₀ values of other compounds disclosed herein are at least 10 μM against c-ABL enzyme.

### Cell Culture

BaF3 cells (parental or transfected with the following: wild type p210 BCR-ABL and T315I p210 BCR-ABL was obtained from Professor Richard Van Etten (New England Medical Center, Boston, Mass.). Briefly, cells were grown in RPMI 1640 supplemented with 10% characterized fetal bovine serum (HyClone, Logan, Utah) at 37 degrees Celsius, 5% CO₂, 95% humidity. Cells were allowed to expand until reaching 80% saturation at which point they were subcultured or harvested for assay use.

### Cell Proliferation Assay

A serial dilution of test compound was dispensed into a 96 well black clear bottom plate (Corning, Corning, N.Y.). For each cell line, three thousand cells were added per well in complete growth medium. Plates were incubated for 72 hours at 37 degrees Celsius, 5% CO₂, 95% humidity. At the end of the incubation period Cell Titer Blue (Promega, Madison, Wis.) was added to each well and an additional 4.5 hour incubation at 37 degrees Celsius, 5% CO₂, 95% humidity was performed. Plates were then read on a BMG Fluostar Optima (BMG, Durham, N.C.) using an excitation of 544 nM and an emission of 612 nM. Data was analyzed using Prism software (Graphpad, San Diego, Calif.) to calculate IC50's.

TABLE 2
Biological Data Summary. Whole Cell Antiproliferation
IC₅₀ values of compounds of Formula I.

| | | Ba/F3 p210 T315I |
|---|---|---|
| | Ba/F3 p210 whole cell | whole cell proliferation |
| Example | proliferation assay | assay |
| 1 | +++ | +++ |
| 2 | +++ | +++ |
| 3 | +++ | +++ |
| 4 | +++ | +++ |
| 5 | +++ | +++ |
| 6 | +++ | +++ |
| 7 | +++ | ++ |
| 8 | +++ | +++ |
| 9 | +++ | ++ |
| 10 | +++ | ++ |
| 11 | +++ | +++ |
| 12 | +++ | +++ |
| 13 | +++ | +++ |
| 14 | +++ | +++ |
| 15 | +++ | +++ |
| 16 | +++ | ++ |
| 17 | +++ | +++ |
| 18 | +++ | +++ |
| 19 | +++ | + |
| 20 | +++ | ++ |
| 21 | +++ | +++ |
| 22 | +++ | +++ |
| 23 | +++ | ++ |
| 24 | +++ | ‡ |
| 25 | +++ | +++ |
| 26 | n/a | n/a |
| 27 | +++ | +++ |
| 28 | +++ | ++ |
| 29 | ++ | ‡ |
| 30 | +++ | ++ |
| 31 | +++ | +++ |
| 32 | +++ | +++ |
| 33 | ++ | ++ |
| 34 | +++ | ++ |
| 35 | ++ | ‡ |
| 36 | + | ‡ |
| 37 | +++ | +++ |
| 38 | ++ | ‡ |
| 39 | ++ | + |
| 40 | ++ | ++ |
| 41 | ++ | ++ |
| 42 | +++ | +++ |

+++ = <0.1 μM;
++ = <1.0 μM;
+ = <10 μM;
‡ <100 μM;
n/a = not available

1. A method of modulating a kinase activity of a wild-type kinase species, oncogenic forms thereof, aberrant fusion proteins thereof and polymorphs of any of the foregoing, comprising the step of contacting said species with a compound of formula Ia: 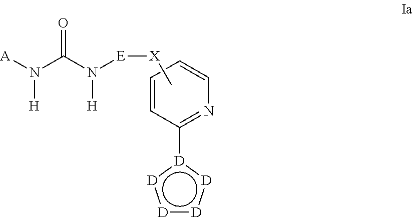
wherein the pyridine ring may be optionally substituted with one or more R20 moieties;
each D is individually taken from the group consisting of C, CH, C—R20, N—Z3, and N, such that the resultant ring is a pyrazole;
wherein E is selected from the group consisting of phenyl, pyridyl, and pyrimidinyl;
E may be optionally substituted with one or two R16 moieties;
wherein A is a ring system selected from the group consisting of phenyl, naphthyl, cyclopentyl, cyclohexyl, G1, G2, and G3;
G1 is a heteroaryl taken from the group consisting of pyrrolyl, furyl, thienyl, oxazolyl, thiazolyl, isoxazol-4-yl, isoxazol-5-yl, isothiazolyl, imidazolyl, pyrazolyl, oxadiazolyl, thiadiazolyl, triazolyl, tetrazolyl, pyrazinyl, pyridazinyl, triazinyl, pyridinyl, and pyrimidinyl;
G2 is a fused bicyclic heteroaryl taken from the group consisting of indolyl, indolinyl, isoindolyl, isoindolinyl, indazolyl, benzofuranyl, benzothienyl, benzothiazolyl, benzothiazolonyl, benzoxazolyl, benzoxazolonyl, benzisoxazolyl, benzisothiazolyl, benzimidazolyl, benzimidazolonyl, benztriazolyl, imidazopyridinyl, pyrazolopyridinyl, imidazolonopyridinyl, thiazolopyridinyl, thiazolonopyridinyl, oxazolopyridinyl, oxazolonopyridinyl, isoxazolopyridinyl, isothiazolopyridinyl, triazolopyridinyl, imidazopyrimidinyl, pyrazolopyrimidinyl, imidazolonopyrimidinyl, thiazolopyridiminyl, thiazolonopyrimidinyl, oxazolopyridiminyl, oxazolonopyrimidinyl, isoxazolopyrimidinyl, isothiazolopyrimidinyl, triazolopyrimidinyl, dihydropurinonyl, pyrrolopyrimidinyl, purinyl, pyrazolopyrimidinyl, phthalimidyl, phthalimidinyl, pyrazinylpyridinyl, pyridinopyrimidinyl, pyrimidinopyrimidinyl, cinnolinyl, quinoxalinyl, quinazolinyl, quinolinyl, isoquinolinyl, phthalazinyl, benzodioxyl, benzisothiazoline-1,1,3-trionyl, dihydroquinolinyl, tetrahydroquinolinyl, dihydroisoquinolyl, tetrahydroisoquinolinyl, benzoazepinyl, benzodiazepinyl, benzoxapinyl, and benzoxazepinyl;
G3 is a heterocyclyl taken from the group consisting of oxetanyl, azetadinyl, tetrahydrofuranyl, pyrrolidinyl, oxazolinyl, oxazolidinyl, imidazolonyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl;
the A ring may be optionally substituted with one or two R2 moieties;
X is selected from the group consisting of —O—, —S(CH₂)ₙ—, —N(R3)(CH₂)ₙ—, —(CH₂)ₚ—, and wherein the carbon atoms of —(CH₂)ₙ—, —(CH₂)ₚ—, of X may be further substituted by oxo or one or more C1-C6alkyl moieties;
when A, G1, G2 or G3 has one or more substitutable sp2-hybridized carbon atoms, each respective sp2 hybridized carbon atom may be optionally substituted with a Z1 substituent;
when A, G1, G2 or G3 has one or more substitutable sp3-hybridized carbon atoms, each respective sp3 hybridized carbon atom may be optionally substituted with a Z2 substituent;
when A, G1, G2 or G3 has one or more substitutable nitrogen atoms, each respective nitrogen atom may be optionally substituted with a Z4 substituent;
each Z1 is independently and individually selected from the group consisting of C1-6alkyl, branched C3-C7alkyl, C3-C8cycloalkyl, halogen, fluoroC1-C6alkyl wherein the alkyl moiety can be partially or fully fluorinated, cyano, C1-C6alkoxy, fluoroC1-C6alkoxy wherein the alkyl moiety can be partially or fully fluorinated, —(CH₂)ₙOH, oxo, C1-C6alkoxyC1-C6alkyl, (R4)₂N(CH₂)ₙ—, (R3)₂N(CH₂)ₙ—, (R4)₂N(CH₂)_{q}N(R4)(CH₂)ₙ—, (R4)₂N(CH₂)_{q}O(CH₂)ₙ—, (R3)₂NC(O)—, (R4)₂NC(O)—, (R4)₂NC(O)C1-C6alkyl-, —(R4)NC(O)R8, C1-C6alkoxycarbonyl-, -carboxyC1-C6alkyl, C1-C6alkoxycarbonylC1-C6alkyl-, (R3)₂NSO₂—, —SOR3, (R4)₂NSO₂—, —N(R4)SO₂R8, —O(CH₂)_{q}OC1-C6alkyl, —SO₂R3, —SOR4, —C(O)R8, —C(O)R6, —C(═NOH)R6, —C(═NOR3)R6, —(CH₂)ₙN(R4)C(O)R8, —N(R3)(CH₂)_{q}O-alkyl, —N(R3)(CH₂)_{q}N(R4)₂, nitro, —CH(OH)CH(OH)R4, —C(═NH)N(R4)₂, —C(═NOR3)N(R4)₂, —NHC(═NH)R8, R17 substituted G3, R17 substituted pyrazolyl and R17 substituted imidazolyl;
in the event that Z1 contains an alkyl or alkylene moiety, such moieties may be further substituted with one or more C1-C6alkyls;
each Z2 is independently and individually selected from the group consisting of aryl, C1-C6alkyl, C3-C8cycloalkyl, branched C3-C7alkyl, hydroxyl, hydroxyC1-C6alkyl-, cyano, (R3)₂N—, (R4)₂N—, (R4)₂NCl—C6alkyl-, (R4)₂NC2-C₆alkylN(R4)(CH₂)ₙ—, (R4)₂NC2-C6alkylO(CH₂)ₙ—, (R3)₂NC(O)—, (R4)₂NC(O)—, (R4)₂NC(O)—C1-C6alkyl-, carboxyl, -carboxyC1-C6alkyl, C1-C6alkoxycarbonyl-, C1-C6alkoxycarbonylC1-C6alkyl-, (R3)₂NSO₂—, (R4)₂NSO₂—, —SO₂R8, —(CH₂)ₙN(R4)C(O)R8, —C(O)R8, ═O, ═NOH, and ═N(OR6);
in the event that Z2 contains an alkyl or alkylene moiety, such moieties may be further substituted with one or more C1-C6alkyls;
each Z3 is independently and individually selected from the group consisting of H, C1-C6alkyl, branched C3-C7alkyl, C3-C8cycloalkyl, fluoroC1-C6alkyl wherein the alkyl moiety can be partially or fully fluorinated, hydroxyC2-C6alkyl-, C1-C6alkoxycarbonyl-, —C(O)R8, R5C(O)(CH₂)ₙ—, (R4)₂NC(O)—, (R4)₂NC(O)C1-C6alkyl-, R8C(O)N(R4)(CH₂)_{q}—, (R3)₂NSO₂—, (R4)₂NSO₂—, —(CH₂)_{q}N(R3)₂, and —(CH₂)_{q}N(R4)₂;
each Z4 is independently and individually selected from the group consisting of C1-C6alkyl, branched C3-7alkyl, hydroxyC2-C6alkyl-, C1-C6alkoxyC2-C6alkyl-, (R4)₂N—C2-C6alkyl-, (R4)₂N—C2-C6alkylN(R4)-C2-C6alkyl-, (R4)₂N—C2-C6alkyl-O—C2-C6alkyl-(R4)₂NC(O)C1-C6alkyl-, carboxyC1-C6alkyl, C1-C6alkoxycarbonylC1-C6alkyl-, —C2-C6alkylN(R4)C(O)R8, R8-C(═NR3)-, —SO₂R8, and —COR8;
in the event that Z4 contains an alkyl or alkylene moiety, such moieties may be further substituted with one or more C1-C6alkyls;
each R2 is selected from the group consisting of H, C1-C6alkyl, branched C3-C8alkyl, R19 substituted C3-C8cycloalkyl-, fluoroC1-C6alkyl- wherein the alkyl is fully or partially fluorinated, halogen, cyano, C1-C6alkoxy-, and fluoroC1-C6alkoxy- wherein the alkyl group is fully or partially fluorinated, hydroxyl substituted C1-C6alkyl-, hydroxyl substituted branched C3-C8alkyl-, cyano substituted C1-C6alkyl-, cyano substituted branched C3-C8alkyl-, (R3)₂NC(O)C1-C6alkyl-, and (R3)₂NC(O)C3-C8 branched alkyl-;
wherein each R3 is independently and individually selected from the group consisting of H, C1-C6alkyl, branched C3-C7alkyl, and C3-C8cycloalkyl;
each R4 is independently and individually selected from the group consisting of H, C1-C6alkyl, hydroxyC1-C6alkyl-, dihydroxyC1-C6alkyl-, C1-C6alkoxyC1-C6alkyl-, branched C3-C7alkyl, branched hydroxyC1-C6alkyl-, branched C1-C6alkoxyC1-C6alkyl-, branched dihydroxyC1-C6alkyl-, —(CH₂)ₚN(R7)₂, —(CH₂)ₚC(O)N(R7)₂, —(CH₂)ₙC(O)OR3, and R19 substituted C3-C8cycloalkyl-;
each R5 is independently and individually selected from the group consisting of 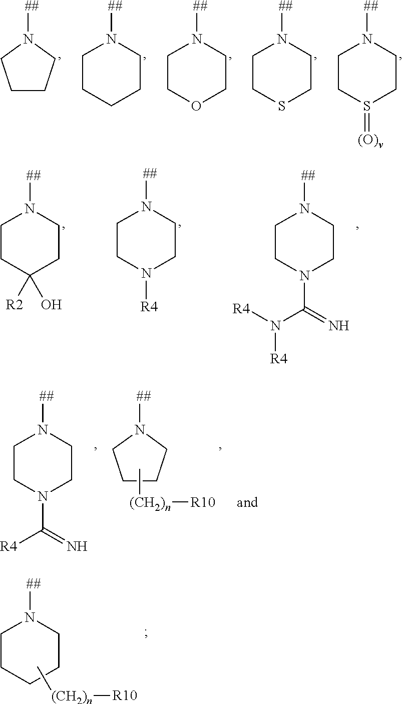
and wherein the symbol (##) is the point of attachment to Z3;
each R6 is independently and individually selected from the group consisting of C1-C6alkyl, branched C3-C7alkyl, and R19 substituted C3-C8cycloalkyl-;
each R7 is independently and individually selected from the group consisting of H, C1-C6alkyl, hydroxyC2-C6alkyl-, dihydroxyC2-C6alkyl-, C1-C6alkoxyC2-C6alkyl-, branched C3-C7alkyl, branched hydroxyC2-C6alkyl-, branched C1-C6alkoxyC2-C6alkyl-, branched dihydroxyC2-C6alkyl-, —(CH₂)ₙC(O)OR3, R19 substituted C3-C8cycloalkyl- and —(CH₂)ₙR17;
each R8 is independently and individually selected from the group consisting of C1-C6alkyl, branched C3-C7alkyl, fluoroC1-C6alkyl- wherein the alkyl moiety is partially or fully fluorinated, R19 substituted C3-C8cycloalkyl-, —OH, C1-C6alkoxy, —N(R3)₂, and —N(R4)₂;
each R10 is independently and individually selected from the group consisting of —CO₂H, —CO₂C1-C6alkyl, —C(O)N(R4)₂, OH, C1-C6alkoxy, and —N(R4)₂;
each R16 is independently and individually selected from the group consisting of H, C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, R3 substituted C2-C3alkynyl- and nitro;
each R17 is independently and individually selected from the group consisting of H, C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, and nitro;
each R19 is independently and individually selected from the group consisting of H, OH and C1-C6alkyl;
each R20 is independently and individually selected from the group consisting of C1-C6alkyl, branched C3-C7alkyl, R19 substituted C3-C8cycloalkyl-, halogen, fluoroC1-C6alkyl- wherein the alkyl moiety can be partially or fully fluorinated, cyano, hydroxyl, C1-C6alkoxy, fluoroC1-C6alkoxy- wherein the alkyl moiety can be partially or fully fluorinated, —N(R3)₂, —N(R4)₂, —N(R3)C(O)R3, —C(O)N(R3)₂ and nitro and wherein two R4 moieties independently and individually taken from the group consisting of C1-C6alkyl, branched C3-C6alkyl, hydroxyalkyl-, and alkoxyalkyl and attached to the same nitrogen heteroatom may cyclize to form a C3-C7 heterocyclyl ring;
k is 0 or 1; n is 0-6; p is 1-4; q is 2-6; r is 0 or 1; t is 1-3; v is 1 or 2; m is 0-2;
or a pharmaceutically acceptable salt, a stereoisomer, a regioisomer, or a tautomer of such compounds.

2. A method of treating mammalian disease wherein the disease etiology or progression is at least partially mediated by the kinase activity of c-ABL kinase, BCR-ABL kinase, FLT-3 kinase, VEGFR-2 kinases, c-MET kinase, PDGFR-alpha kinase, PDGFR-beta kinase, HER-1 kinase, HER-2 kinase, HER-3 kinase, HER-4 kinase, FGFR kinases, c-KIT kinase, RET kinase, c-FMS kinase, oncogenic forms thereof, aberrant fusion proteins thereof and polymorphs of any of the foregoing, comprising the step of administering to the mammal a therapeutically effective amount of a pharmaceutical composition comprising a compound of formula Ia.

3. A method of claim 2 wherein said kinase is selected from the group consisting of BCR-ABL fusion protein kinases p210, BCR-ABL fusion protein kinases p190, BCR-ABL fusion protein kinases bearing the T315I gatekeeper mutant in the ABL kinase domain of p210, BCR-ABL fusion protein kinases bearing the T315I gatekeeper mutant in the ABL kinase domain of p190, and other BCR-ABL polymorphs of any of the foregoing kinases.

4. The method of claim 3, wherein said BCR-ABL fusion protein kinases p210 have Seq. IDs 3 & 4, wherein said BCR-ABL fusion protein kinase p190 has Seq. ID 5, wherein said BCR-ABL fusion protein kinases p210 bearing the T315I mutation in the ABL kinase domain have Seq. IDs 6 & 7, and wherein said BCR-ABL fusion protein kinase p190 bearing the T315I mutation in the ABL kinase domain has Seq. ID 8.

5. The method of claim 2 wherein said kinase is selected from the group consisting of c-KIT protein kinase, PDGFR-alpha kinase, PDGFR-beta kinase, c-FMS kinase, and any fusion protein, mutation and polymorph of any of the foregoing.

6. The method of claim 2 wherein said kinase is selected from the group consisting of c-MET protein kinase, RET kinase, FGFR kinases, HER kinases, and any fusion protein, mutation and polymorph of any of the foregoing.

7. A method of treating an individual suffering from a condition selected from the group consisting of cancer, secondary cancer growth arising from metastasis, hyperproliferative diseases, diseases characterized by hyper-vascularization, inflammation, osteoarthritis, rheumatoid arthritis, respiratory diseases, stroke, systemic shock, immunological diseases, automimmune diseases, bone resorptive diseases, cardiovascular disease and diseases characterized by angiogenesis, comprising the step of administering to such individual a therapeutically effective amount of a pharmaceutical composition comprising a compound of formula Ia.

8. A method of treating an individual suffering from a disease caused by c-ABL kinase, oncogenic forms thereof, aberrant fusion proteins thereof including BCR-ABL kinase and polymorphs thereof; a disease caused by FLT-3 kinase, oncogenic forms thereof, aberrant fusion proteins thereof and polymorphs thereof; a disease caused by cMET kinase, oncogenic forms thereof, aberrant fusion proteins thereof including TPR-MET; a disease caused by KDR kinase or PDGFR kinases; a disease caused by HER kinases, oncogenic forms thereof and polymorphs thereof; a disease caused by RET kinase, oncogenic forms thereof, aberrant fusion proteins thereof; a disease caused by c-FMS kinase, oncogenic forms thereof and polymorphs thereof; a disease caused by a c-KIT kinase, oncogenic forms thereof, aberrant fusion proteins thereof and polymorphs thereof; and diseases caused by any of the foregoing kinases, oncogenic forms thereof, and aberrant fusion proteins thereof, including but not limited to, chronic myelogenous leukemia, acute lymphocytic leukemia, acute myeloid leukemia, other myeloproliferative disorders, a disease caused by metastasis of primary solid tumors to secondary sites, glioblastomas, ovarian cancer, pancreatic cancer, prostate cancer, lung cancers, mesothelioma, hypereosinophilic syndrome, a disease caused or maintained by pathological vascularization, ocular diseases characterized by hyperproliferation leading to blindness including various retinopathies, i.e. diabetic retinopathy and age-related macular degeneration, non small cell lung cancer, breast cancers, kidney cancers, colon cancers, cervical carcinomas, papillary thyroid carcinoma, melanomas, autoimmune diseases including rheumatoid arthritis, multiple sclerosis, lupus, asthma, human inflammation, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic obstructive pulmonary disease, bone resorptive diseases, bone cancer, graft-versus-host reaction, Chron's disease, ulcerative colitis, inflammatory bowel disease, pyresis, gastrointestinal stromal tumors, mastocytosis, mast cell leukemia, and combinations thereof, comprising the step of administering to such individual a therapeutically effective amount of a pharmaceutical composition comprising a compound of formula Ia.

9. The method of claim 8, said compound being administered by a method selected from the group consisting of oral, parenteral, inhalation, and subcutaneous.

10. The method of claim 7 or 8, wherein the pharmaceutical composition further comprises at least one other therapeutic agent.

11. The method of claim 10, wherein the at least one other therapeutic agent is useful for treating cancer.

12. The method of claim 11, wherein the other therapeutic agent is selected from the group consisting of imatinib, nilotinib, dasatinib, and bosutinib.

13. The method of claim 12, wherein the other therapeutic agent is imatinib.

14. The method of claim 10, wherein the at least one other therapeutic agent is useful for treating autoimmune diseases or inflammatory diseases.

15. The method of claim 14, wherein the other therapeutic agent is selected from the group consisting of methotrexate or other anti-folate agent.

16. The method of claim 14, wherein the other therapeutic agent is an anti-TNF agent.

17. The method of claim 16, wherein the other therapeutic agent is selected from the group consisting Humira®, Enbrel®, and Remicade®.
